(19) United States
(12) Patent Application Publication
(10) Pub. No.: US 2026/0015664 A1
(43) Pub. Date: Jan. 15, 2026

(54) METHODS FOR DETERMINATION AND MONITORING OF TRANSPLANT REJECTION BY MEASURING RNA

(71) Applicant: Natera, Inc., San Carlos, CA (US)

(72) Inventors: Alain Mir, Castro Valley, CA (US); Anastasia Butskova, San Carlos, CA (US); Aaron Bruns, San Mateo, CA (US); Mathieu Chauleau, Walnut Creek, CA (US); Ebad AHMED, San Jose, CA (US); Paul VAN HUMMELEN, San Francisco, CA (US)

(73) Assignee: Natera, Inc., San Carlos, CA (US)

(21) Appl. No.: 18/866,747

(22) PCT Filed: Jun. 15, 2023
(86) PCT No.: PCT/US2023/025430

§ 371 Date: Nov. 18, 2024

Related U.S. Application Data
(60) Provisional application No. 63/352,542, filed on Jun. 15, 2022.
(60) Provisional application No. 63/404,412, filed on Sep. 7, 2022.

(51) Int. Cl. C12Q 1/6883; C12Q 1/48; C12Q 1/6806

(52) U.S. Cl. CPC C12Q 1/6883; C12Q 1/485; C12Q 1/6806; C12Q 2600/178

(57) ABSTRACT

The present disclosure provides methods for preparation and analysis of biological samples of transplant recipients or subjects suffering from a disease or disorder, wherein the methods comprise extracting fragmented or intact RNA (such as mRNA or miRNA) derived from sample of the transplant recipient or subject suffering from a disease or disorder, wherein the extracted RNA comprises target RNA molecules preselected to enable assessment of transplant rejection. The detection of the target RNA molecules and total amount of RNA derived from the donor organ can be used to determine and/or monitoring transplant rejection.

### BACKGROUND

Rapid detection of graft injury and/or rejection remains a challenge for transplant recipients. Conventional biopsy-based tests are invasive and costly and possibly lead to late diagnosis of transplant injury and/or rejection. Therefore, there is a need for a non-invasive test for transplant injury and/or rejection that is more sensitive and more specific than conventional biopsy-based tests.

Non-invasive tests can also be used to test, screen, or monitor various diseases and disorders, including cancer, diseases and disorders associated with women's health, dysfunctional organs, or cardiovascular disorders. Cancer is one of the leading causes of death; thus, early diagnosis and treatment of cancer is important, since it can improve the patient's outcome (such as by increasing the probability of remission and the duration of remission), and reduce the amount and/or number of treatments (such as chemotherapeutic agents or radiation) needed to eliminate the cancer. Non-invasive tests are also important for providing early detection and subsequent monitoring of diseases and conditions in general, including in particular cardiovascular disorders such as preeclampsia or congenital heart disease.

Thus, improved methods are needed to accurately diagnose, screen, test, and monitor disease (such as transplant rejection or cancer) or disease status, and/or treatment responsiveness at an early stage.

### SUMMARY

In at least one aspect, the present disclosure relates to a method of preparing a composition of amplified nucleic acids derived from a sample obtained from a transplant recipient useful for determining transplant rejection, comprising: extracting fragmented or intact RNA (such as mRNA) derived from sample of the transplant recipient, wherein the extracted RNA comprises donor- and/or recipient-derived RNA; preparing a composition of nucleic acids from the RNA by performing reverse transcription of the RNA to produce complementary DNA (cDNA) followed by targeted amplification to obtain amplicons of a plurality of target RNA molecules indicative of transplant rejection or organ health, and measuring the amount measuring the amount of the target RNA molecules in the sample based on the amplicons obtained in step b) to quantify the amount of donor-derived target RNA molecules, thereby determining transplant rejection.

In one aspect, the present disclosure relates to a method of preparing a composition of amplified nucleic acids derived from a sample obtained from a transplant recipient useful for determining transplant rejection, wherein the sample comprises donor- and/or recipient-derived RNA;
a) preparing a composition of nucleic acids from the RNA by performing reverse transcription of the RNA to produce complementary DNA (cDNA) followed by amplification to obtain amplicons;
b) measuring the amount of RNA in the sample based on the amplicons obtained in step (b) to quantify the amount of donor-derived RNA and/or the amount of a plurality of target RNA molecules indicative of transplant rejection or organ health; and
c) determining transplant rejection or organ health based on (i) whether the amount of donor derived RNA or a function thereof exceeds a cutoff threshold indicating transplant rejection, and (ii) whether the amount of a plurality of target RNA molecules indicative of transplant rejection or organ health or a function thereof exceeds a cutoff threshold indicating transplant rejection.

In some exemplary embodiments, the step of determining transplant rejection comprises determining whether the amount of donor derived target RNA molecules or a function thereof exceeds a cutoff threshold indicating transplant rejection.

In some exemplary embodiments, the methods herein further comprise utilizing CRISPR-Cas to target and deplete contaminating or overabundant nucleic acid species in the sample, thereby increasing the fraction of desired reads that map to a target loci of interest per sample and the sample throughput per sequencing run

In some exemplary embodiments, the sample comprises whole blood or hemolysis tainted blood, serum or plasma samples, and wherein a plurality of guide RNAs are used to target a plurality of loci in the same reaction to deplete contaminating or overabundant nucleic acid, thereby increasing a detection rate of the target loci.

In some exemplary embodiments, the contaminating or overabundant nucleic acid species comprise hemoglobin mRNA, tRNA, and rRNA, and miR-451, miR-144, and miR-486.

In some exemplary embodiments, the methods herein further comprise depleting adaptor dimers, primer dimers, unwanted ligation products from the composition of amplified nucleic acids comprising target loci, thereby increasing the fraction of desired reads that map to a target loci of interest per sample and the sample throughput per sequencing run.

In some exemplary embodiments, Cas9/Cas12a is utilized to remove nucleic acid species after reverse transcription of RNA and before multiplex amplification.

In some exemplary embodiments, Cas9/Cas12a is utilized to remove nucleic acid species after 1-10 cycles of multiplex amplification of the complementary DNA.

In some exemplary embodiments, the contaminating or overabundant nucleic acid species are RNA, and wherein Cas13 is used to remove the contaminating or overabundant RNA species from the sample.

In some exemplary embodiments, the methods herein further comprise:
measuring the amount of donor-derived cell-free DNA in a sample obtained from the transplant recipient, extracting cell-free DNA from the sample obtained from the transplant recipient, wherein the extracted cell-free DNA comprises donor-derived cell-free DNA and recipient-derived cell-free DNA;
performing targeted amplification of the extracted DNA at 50-50,000 target loci in a single reaction volume;
sequencing the amplified DNA by high-throughput sequencing to obtain sequencing reads and quantifying the amount of donor-derived cell-free DNA based on the sequencing reads, determining transplant rejection based on whether the amount of donor-derived cell-free DNA or a function thereof exceeds a cutoff threshold of cell-free DNA amount that indicates transplant rejection, wherein transplant rejection is determined based on whether both the amount of donor-derived RNA and the amount of donor derived cell-free DNA or function thereof exceeds a cutoff threshold that indicates transplant rejection.

In some exemplary embodiments, the amount of donor-derived mRNA is determined by using ratiometric and/or machine learning-artificial intelligence comparisons at a single or a plurality of time points.

In some exemplary embodiments, the sample comprises blood, plasma, serum, CSF or urine.

In some exemplary embodiments, the transplant recipient is a human subject.

In some exemplary embodiments, the transplant recipient has received a plurality of transplanted organs selected from pancreas, kidney, liver, lung, heart, intestinal, thymus or uterus.

In some exemplary embodiments, the one or more transplanted organs are from the same transplant donors.

In some exemplary embodiments, the one or more transplanted organs are from more multiple different transplant donors.

In some exemplary embodiments, the transplant recipient has received simultaneous transplantation of more than one organ.

In some exemplary embodiments, step (b) comprises preferentially enriching the target RNA molecules.

In some exemplary embodiments, the RNA comprises biomarkers of an increased immune response, or a decreased immune response.

In some exemplary embodiments, the methods herein further comprise:

measuring the amounts of RNA longitudinally for the same transplant recipient; determining a longitudinal change in the amount of RNA.

In some exemplary embodiments, the RNA is cell-free RNA. In some exemplary embodiments, the cell-free RNA is derived from exosomes or microvesicles. In some exemplary embodiments, the RNA is cell-free RNA. In some exemplary embodiments, the cell-free RNA is derived from exosomes or microvesicles. In some exemplary embodiments, the RNA is small messenger RNA (mRNA). In some exemplary embodiments, the RNA is small noncoding RNA (sncRNA). In some exemplary embodiments, the sncRNA comprises micro RNA (miRNA), piwi-interacting RNA (piRNA), small nucleolar RNA (snoRNA), small nuclear RNA (snRNA), or miscellaneous RNA (miscRNA).

In some exemplary embodiments, step (b) comprises amplification of at least 2, at least 5, at least 10, at least 20, at least 30, at least 50, at least 100 target RNA molecules, from 2-10, 200-100, 50-500, or 50-2000 target RNA molecules, using at least at least 2, at least 5, at least 10, at least 20, at least 30, at least 50, at least 100 target RNA molecules, from 2-10, 200-100, 50-500, or 50-2000 pairs of forward and reverse PCR primers.

In some exemplary embodiments, the amount of RNA or cell-free DNA is measured by a quantitative PCR method, and wherein the quantitative PCR method comprises real-time PCR or digital PCR.

In some exemplary embodiments, the amount of RNA or cell-free DNA is measured by massively multiplex PCR (mmPCR) to obtain amplicons comprising biomarkers, and sequencing of the amplicons.

In some exemplary embodiments, the amount of RNA or cell-free DNA is measured by using microarray.

In some exemplary embodiments, the amount of RNA or cell-free DNA is measured by using molecular barcodes and microscopic imaging (such as NanoString nCounter®).

In some exemplary embodiments, the cutoff threshold is an estimated percentage of donor-derived RNA out of total RNA or a function thereof.

In some exemplary embodiments, the cutoff threshold is an estimated percentage of donor-derived cell-free DNA out of total cell-free DNA or a functional thereof.

In some exemplary embodiments, the cutoff threshold is proportional to an absolute donor-derived RNA or cell-free DNA concentration.

In some exemplary embodiments, a rejection risk for the transplant recipient can be determined based on the amount of donor-derived RNA and/or amount of cell-free DNA.

In some exemplary embodiments, the rejection risk for the transplant recipient is determined using machine learning such as Mann-Whitney test, Benjamini-Hochberg (BH) Procedure, logistic LASSO regression, Recursive Feature Elimination (RFE)—support vector machine (SVM), RFe-Random Forest, or gradient boosting.

In some exemplary embodiments, the machine learning analysis comprises, Mann-Whitney test, Benjamini-Hochberg (BH) Procedure, logistic LASSO regression, Recursive Feature Elimination (RFE)—support vector machine (SVM), RFE-Random Forest, or gradient boosting, and further incorporates one or more parameters selected from time post-transplantation, age of transplant recipient and/or transplant donor, gender of transplant recipient and/or transplant donor, the amount of RNA in the sample, or the amount of total cell-free DNA in the sample or a function thereof.

In some exemplary embodiments, the sample is obtained from the transplant recipient less than 18 months post-transplantation.

In another aspect, the present disclosure relates to a method of preparing a composition of nucleic acids derived from a sample obtained from a transplant recipient useful for determination of transplant rejection, comprising:
(a) extracting RNA from the sample obtained from the transplant recipient;
(b) preparing a composition of nucleic acids from the extracted RNA, wherein the nucleic acids comprise a plurality of biomarkers indicative of transplant rejection;
(c) measuring the amount of RNA comprising a plurality of biomarker indicative of transplant rejection in the sample obtained from the transplant recipient; and
(d) determining whether the amount of recipient-derived RNA comprising biomarkers indicative of transplant rejection exceeds a cutoff threshold or a function thereof. In some exemplary embodiments, step (b) comprises preferentially enriching the RNA at a plurality of biomarkers indicative of transplant rejection.

In some exemplary embodiments, preferentially enriching the RNA at the plurality of biomarkers comprises: obtaining a set of hybrid capture probes; hybridizing the hybrid capture probes to the sncRNA in the sample; and physically separating the hybridized sncRNA from the sample of sncRNA from the unhybridized sncRNA from the sample.

In some exemplary embodiments, the nucleic acids comprising the plurality of biomarkers indicative of transplant rejection are determined by hybridization of the extracted RNA to a plurality of probes, or by reverse transcription and targeted amplification of the extracted RNA in a single reaction volume, and sequencing the amplified RNA by high-throughput sequencing to obtain sequencing reads.

In some exemplary embodiments, the RNA comprises biomarkers of an increased immune response, or a decreased immune response.

In some exemplary embodiments, the transplant recipient is a human subject.

In some exemplary embodiments, the transplant recipient has received one or more transplanted organs selected from pancreas, kidney, liver, heart, lung, intestinal, thymus, and uterus.

In another aspect, the present disclosure relates to a method of administrating immunosuppressive therapy in a transplant recipient, comprising:
(a) identifying a plurality of RNA biomarkers in a sample obtained from the transplant recipient; and
(b) titrating the dosage of an immunosuppressive therapy according to the RNA biomarkers.

In another aspect, the present disclosure relates to a method of administrating immunosuppressive therapy in a transplant recipient, comprising:
(a) identifying a plurality of RNA biomarkers in a sample obtained from the transplant recipient, and detecting an amount of donor-derived RNA; and
(b) titrating the dosage of an immunosuppressive therapy according to the RNA biomarkers and the amount of donor-derived RNA.

In some exemplary embodiments, the RNA comprises a biomarker indicating an immune response.

In some exemplary embodiments, the transplant recipient is a human subject.

In some exemplary embodiments, the transplant recipient has received one or more transplanted organs selected from pancreas, kidney, liver, heart, lung, intestinal, thymus, and uterus.

In some exemplary embodiments, the methods herein further comprise:
measuring the amount of donor-derived cell-free DNA in a sample obtained from the transplant recipient; and wherein the amount of donor-derived cell-free DNA is measured by:
extracting cell-free DNA from the sample obtained from the transplant recipient, wherein the extracted cell-free DNA comprises donor-derived cell-free DNA and recipient-derived cell-free DNA;
performing targeted amplification of the extracted DNA at 10-50,000 target loci in a single reaction volume;
sequencing the amplified DNA by high-throughput sequencing to obtain sequencing reads and quantifying the amount of donor-derived cell-free DNA based on the sequencing reads, titrating the dosage of an immunosuppressive therapy according to the amount of RNA or a function thereof and the amount of cell-free DNA or a function thereof

In some exemplary embodiments, the methods herein further comprise:
measuring the amounts of RNA longitudinally for the same transplant recipient; determining a longitudinal change in RNA comprising biomarkers indicative of a reduced immune response or an increased immune response.

In some exemplary embodiments, the methods herein further comprise: measuring the amounts of cell-free DNA longitudinally for the same transplant recipient, and determining a longitudinal change in the amount of donor-derived cell-free DNA or a function thereof.

In some exemplary embodiments, the methods herein further comprise: increasing the dosage of immunosuppressive therapy if the transplant recipient has a longitudinally increased amount of RNA comprising biomarkers indicative of transplant rejection, and/or a longitudinally increased amount of donor-derived cell-free DNA.

In some exemplary embodiments, the methods herein further comprise: decreasing the dosage of immunosuppressive therapy if the transplant recipient has a longitudinally decreased amount of RNA comprising biomarkers indicative of transplant rejection, and/or a longitudinally decreased amount of donor-derived cell-free DNA.

In some exemplary embodiments, the RNA comprises biomarkers indicating increased immune response.

In another aspect, the present disclosure relates to a method of preparing a composition of amplified RNA derived from a sample obtained from a subject useful for monitoring or detecting a disease or a disease status, comprising:
a) extracting RNA from the blood, plasma, serum or urine sample of the subject, wherein the RNA comprises biomarkers;
(b) preparing a composition of amplified nucleic acids from the extracted RNA by performing amplification of the extracted RNA, and sequencing the amplified nucleic acids by high-throughput sequencing to obtain sequencing reads;
(c) measuring the amount of RNA comprising the plurality of biomarkers in the sample obtained from the subject based on the sequencing reads obtained in step (b); and
(d) determining whether the amount of RNA comprising biomarkers indicative of the disease or disease status exceeds a cutoff threshold or function thereof, thereby determining the disease or disease status in the subject.

In some exemplary embodiments, the methods herein further comprise:
measuring the amount of donor-derived cell-free DNA in a sample derived from the subject, extracting cell-free DNA from the sample of the subject, wherein the extracted cell-free DNA comprises biomarkers indicative of the disease or the disease status;
performing targeted amplification of the extracted DNA at 10-50,000 biomarkers in a single reaction volume;
sequencing the amplified DNA by high-throughput sequencing to obtain sequencing reads and quantifying the amount of cell-free DNA that comprises biomarkers indicative of the disease or the disease status based on the sequencing reads; and determining the disease or disease status based both on whether the amount of cell-free DNA that comprises biomarkers indicative of the disease or disease status exceeds a cutoff threshold or a function thereof, and whether the amount of RNA comprising biomarkers indicative of the disease or disease status exceeds a cutoff threshold or function thereof.

In another aspect, the present disclosure relates to a method of administrating a therapy to a subject suffering from a disease, comprising:
(a) measuring the amount of RNA that comprises biomarkers indicative of the disease or a status of the disease in a sample obtained from the subject; and
(b) titrating the dosage of the therapy according to the amount of RNA that comprises biomarkers indicative of the disease or a status of the disease or a function thereof.

In some exemplary embodiments, the methods herein further comprise:

measuring the amount of cell-free DNA in a sample obtained from the subject; and wherein the amount of donor-derived cell-free DNA is measured by:

extracting cell-free DNA from the sample obtained from the subject, wherein the extracted cell-free DNA comprises biomarkers of the disease or the disease status;
performing targeted amplification of the extracted DNA at 200-50,000 biomarkers in a single reaction volume;
sequencing the amplified DNA by high-throughput sequencing to obtain sequencing reads and quantifying the amount of cell-free DNA comprising biomarkers of the disease or disease status based on the sequencing reads, and titrating the dosage of the therapy both according to the amount of cell-free DNA comprising biomarkers of the disease or disease status or a function thereof, and the amount of RNA that comprises biomarkers indicative of the disease or a status of the disease or a function thereof.

In some exemplary embodiments, the methods herein further comprise:
measuring the amounts of RNA comprising biomarkers indicative of the disease or as status of the disease longitudinally for the same subject, and determining a longitudinal change in the amount of RNA comprising biomarkers indicative of the disease or as status of the disease.

In some exemplary embodiments, the methods herein further comprise:
measuring the amounts of cell-free DNA comprising biomarkers of the disease or disease status longitudinally for the same transplant recipient, and determining a longitudinal change in the amount of donor-derived cell-free DNA comprising biomarkers of the disease or disease status or a function thereof.

In some exemplary embodiments, the methods herein further comprise:
increasing the dosage of the therapy if the subject has a longitudinally increased amount of RNA comprising biomarkers indicative of the disease or disease status, and/or a longitudinally increased amount of cell-free DNA comprising biomarkers indicative of the disease or disease status. In some exemplary embodiments, the sample is a blood, a plasma, a serum, a saliva or a urine sample obtained from the subject. In some exemplary embodiments, the RNA is cell-free RNA. In some exemplary embodiments, the RNA is derived from exosomes or microvesicles. In some exemplary embodiments, the RNA is mRNA. In some exemplary embodiments, the mRNA is selected from a group of mRNA targets with known relevance to organ health. In some exemplary embodiments, the RNA is small noncoding RNA (sncRNA). In some exemplary embodiments, (piRNA), small nucleolar RNA (snoRNA), small nuclear RNA (snRNA), or miscellaneous RNA (miscRNA).

In some exemplary embodiments, the disease or the disease status comprises a cancer, recurrence or metastasis of the cancer, an immune disease or disorder, pre-eclampsia, or congenital heart disease (CHD).

In some exemplary embodiments, the cancer is breast cancer, lung cancer, liver cancer, skin cancer, prostate cancer, bladder cancer, or colorectal cancer.

In some exemplary embodiments, the methods herein further comprise: PCR amplification of at least 2, at least 5, at least 10, at least 20, at least 30, at least 50, at least 100 target RNA molecules, from 2-10, 200-100, 50-500, or 50-2000 target RNA molecules, using at least at least 2, at least 5, at least 10, at least 20, at least 30, at least 50, at least 100 target RNA molecules, from 2-10, 200-100, 50-500, or 50-2000 pairs of forward and reverse PCR primers.

In some exemplary embodiments, the methods herein further comprise: utilizing CRISPR-Cas to target and deplete contaminating or overabundant nucleic acid species in the sample, thereby increasing the fraction of desired reads that map to a target loci of interest per sample and the sample throughput per sequencing run.

In some exemplary embodiments, the sample comprises whole blood or hemolysis tainted blood, serum or plasma samples, and wherein a plurality of guide RNAs are used to target a plurality of loci in the same reaction to deplete contaminating or overabundant nucleic acid, thereby increasing a detection rate of the target loci.

In some exemplary embodiments, contaminating or overabundant nucleic acid species comprise hemoglobin mRNA, tRNA, and rRNA, and miR-451, miR-144, and miR-486.

In some exemplary embodiments, the methods herein further comprise: depleting adaptor dimers, primer dimers, unwanted ligation products from the composition of amplified nucleic acids comprising target loci, thereby increasing the fraction of desired reads that map to a target loci of interest per sample and the sample throughput per sequencing run.

In some exemplary embodiments, Cas9/Cas 12a is utilized to remove nucleic acid species after reverse transcription of RNA and before multiplex amplification.

In some exemplary embodiments, Cas9/Cas 12a is utilized to remove nucleic acid species after 1-10 cycles of multiplex amplification of the complementary DNA.

In some exemplary embodiments, the contaminating or overabundant nucleic acid species are RNA, and wherein Cas13 is used to remove the contaminating or overabundant RNA species from the sample.

In some exemplary embodiments, the RNA is miRNA determined to be of relevance to transplant organ health determined by text mining databases.

In some exemplary embodiments, RNA is miRNA predicted to bind mRNAs known to be relevant for transplant organ health.

In some exemplary embodiments, RNA is miRNA determined to be of relevance to transplant organ health determined by text mining databases, and predicted to bind mRNAs known to be relevant for transplant organ health.

In some exemplary embodiments, the target RNA molecules comprise the miRNA molecules set forth in Tables 2, 3, and/or 4.

In some exemplary embodiments, the target RNA molecules comprise one or more miRNA molecules selected from the group consisting of cel-miR-39-3p, hsa-Let-7a-5p, hsa-Let-7d-3p, hsa-Let-7i-5p, hsa-miR-1224-5p, hsa-miR-125b-5p, hsa-miR-126-3p, hsa-miR-1281, hsa-miR-130a-3p, hsa-mir-135a1-5p, hsa-miR-142-3p, hsa-miR-145-3p, hsa-miR-145-5p, hsa-miR-146a-5p, hsa-miR-155-5p, hsa-miR-15a-5p, hsa-miR-15b-5p, hsa-miR-16-5p, hsa-miR-17-3p, hsa-miR-1825, hsa-miR-186-5p, hsa-miR-18a-5p, hsa-miR-18b-3p, hsa-miR-191-5p, hsa-miR-195-5p, hsa-miR-199a-1-3p, hsa-miR-200b-3p, hsa-miR-203a-3p, hsa-miR-204-5p, hsa-miR-208a-3p, hsa-miR-21-5p, hsa-miR-210-3p, hsa-miR-211-5p, hsa-miR-215-5p, hsa-miR-216a-5p, hsa-miR-223-3p, hsa-miR-23a-3p, hsa-miR-24-3p, hsa-miR-299-5p, hsa-miR-30a-3p, hsa-miR-30c-5p, hsa-miR-30d-5p, hsa-miR-320a-3p, hsa-miR-323a-3p, hsa-miR-3615, hsa-miR-377-3p, hsa-miR-378a-3p, hsa-miR-378h, hsa-miR-382-5p, hsa-miR-411-5p, hsa-miR-423-5p, hsa-miR-4286, hsa-miR-449b-5p, hsa-miR-449c-5p, hsa-miR-451a, hsa-miR-484, hsa-miR-487a-5p, hsa-miR-494-3p, hsa-miR-499a-5p, hsa-miR-500a-3p, hsa-miR-625-5p, hsa-miR-877-5p, hsa-miR-92b-3p, hsa-miR-93-5p, hsa-mir19a-5p, and hsa-mir208a-3p.

In some exemplary embodiments, the target RNA molecules comprise one or more miRNA molecules selected from the group consisting of cel-miR-39-3p, hsa-Let-7a-5p, hsa-Let-7d-3p, hsa-Let-7i-5p, hsa-miR-1224-5p, hsa-miR-125b-5p, hsa-miR-126-3p, hsa-miR-1281, hsa-miR-130a-3p, hsa-mir-135a1-5p, hsa-miR-142-3p, hsa-miR-145-3p, hsa-miR-145-5p, hsa-miR-146a-5p, hsa-miR-155-5p, hsa-miR-15a-5p, hsa-miR-15b-5p, hsa-miR-16-5p, hsa-miR-17-3p, hsa-miR-1825, hsa-miR-186-5p, hsa-miR-18a-5p, hsa-miR-18b-3p, hsa-miR-191-5p, hsa-miR-195-5p, hsa-miR-199a-1-3p, hsa-miR-200b-3p, hsa-miR-203a-3p, hsa-miR-204-5p, hsa-miR-208a-3p, hsa-miR-21-5p, hsa-miR-210-3p, hsa-miR-211-5p, hsa-miR-215-5p, hsa-miR-216a-5p, hsa-miR-223-3p, hsa-miR-23a-3p, hsa-miR-24-3p, hsa-miR-299-5p, hsa-miR-30a-3p, hsa-miR-30c-5p, hsa-miR-30d-5p, hsa-miR-320a-3p, hsa-miR-323a-3p, hsa-miR-3615, hsa-miR-377-3p, hsa-miR-378a-3p, hsa-miR-378h, hsa-miR-382-5p, hsa-miR-411-5p, hsa-miR-423-5p, hsa-miR-4286, hsa-miR-449b-5p, hsa-miR-449c-5p, hsa-miR-451a, hsa-miR-484, hsa-miR-487a-5p, hsa-miR-494-3p, hsa-miR-499a-5p, hsa-miR-500a-3p, hsa-miR-625-5p, hsa-miR-877-5p, hsa-miR-92b-3p, hsa-miR-93-5p, hsa-mir19a-5p, hsa-mir208a-3p, hsa-miR-101-3p, hsa-miR-136-3p, hsa-miR-185-3p, hsa-miR-192-3p, hsa-miR-194-5p, hsa-miR-196b-5p, hsa-miR-214-3p, hsa-miR-339-3p, hsa-miR-4746-5p, hsa-miR-500a-5p, hsa-miR-539-5p, hsa-miR-576-5p, hsa-miR-1-3p, hsa-miR-1277-5p, hsa-miR-139-5p, hsa-miR-146b-5p, hsa-miR-183-5p, hsa-miR-188-5p, hsa-miR-190a-5p, hsa-miR-200a-3p, hsa-miR-205-5p, hsa-miR-2115-3p, hsa-miR-329-3p, hsa-miR-3690, hsa-miR-376a-3p, hsa-miR-376b-3p, hsa-miR-412-5p, hsa-miR-449a, hsa-miR-539-3p, hsa-miR-551a, hsa-miR-582-3p, hsa-miR-628-5p, hsa-miR-629-5p, hsa-miR-642a-5p, hsa-miR-651-5p, hsa-miR-873-5p, hsa-miR-887-3p, and hsa-miR-376c-3p.

In some exemplary embodiments, the target RNA molecules comprise one or more miRNA molecules selected from the group consisting of hsa-miR-101-3p, hsa-miR-136-3p, hsa-miR-17-3p, hsa-miR-185-3p, hsa-miR-192-3p, hsa-miR-194-5p, hsa-miR-196b-5p, hsa-miR-214-3p, hsa-miR-339-3p, hsa-miR-4746-5p, hsa-miR-500a-5p, hsa-miR-539-5p, hsa-miR-576-5p, hsa-miR-1-3p, hsa-miR-1277-5p, hsa-miR-139-5p, hsa-miR-146b-5p, hsa-miR-183-5p, hsa-miR-188-5p, hsa-miR-190a-5p, hsa-miR-195-5p, hsa-miR-200a-3p, hsa-miR-205-5p, hsa-miR-2115-3p, hsa-miR-215-5p, hsa-miR-223-3p, hsa-miR-29b-3p, hsa-miR-329-3p, hsa-miR-3690, hsa-miR-376a-3p, hsa-miR-376b-3p, hsa-miR-412-5p, hsa-miR-449a, hsa-miR-449c-5p, hsa-miR-539-3p, hsa-miR-551a, hsa-miR-582-3p, hsa-miR-628-5p, hsa-miR-629-5p, hsa-miR-642a-5p, hsa-miR-651-5p, hsa-miR-873-5p, hsa-miR-887-3p, and hsa-miR-376c-3p.

In some exemplary embodiments, the target RNA molecules comprise one or more miRNA molecules selected from the group consisting of hsa-miR-92b-5p, hsa-miR-6734-5p, hsa-miR-664a-5p, hsa-miR-576-5p, hsa-miR-539-5p, hsa-miR-500a-5p, hsa-miR-4488, hsa-miR-381-3p, hsa-miR-376c-3p, hsa-miR-339-3p, hsa-miR-185-3p, hsa-miR-17-3p, hsa-miR-1271-5p, and ‘hsa-miR-101.

In some exemplary embodiments, the target RNA molecules comprise one or more miRNA molecules selected from the group consisting of hsa-miR-99b-5p, hsa-miR-660-3p, hsa-miR-500a-5p, hsa-miR-4746-5p, hsa-miR-410-3p, hsa-miR-214-3p, hsa-miR-196b-5p, hsa-miR-194-5p, hsa-miR-192-3p, hsa-miR-185-3p, hsa-miR-17-3p, hsa-miR-136-3p, and hsa-miR-101-3p.

In some exemplary embodiments, the target RNA molecule is hsa-miR-17-3p.

In some exemplary embodiments, the target RNA molecules comprise one or more miRNA molecules selected from the group consisting of hsa-miR-5695, hsa-miR-454-5p, hsa-miR-3912-3p, hsa-miR-363-3p, hsa-miR-27a-5p, hsa-miR-191-5p, hsa-miR-17-3p, hsa-miR-145-5p, and hsa-let-7i-3p.

In some exemplary embodiments, the target RNA molecules comprise one or more miRNA molecules selected from the group consisting of hsa-miR-652-3p, hsa-miR-584-5p, hsa-miR-378a-3p, hsa-miR-338-3p, hsa-miR-320a-3p, hsa-miR-29c-3p, hsa-miR-221-3p, hsa-miR-20a-5p, hsa-miR-199b-3p, hsa-miR-181a-5p, hsa-miR-17-5p, hsa-miR-17-3p, hsa-miR-151a-5p, hsa-miR-151a-3p, hsa-miR-148a-3p, and hsa-miR-143-3p.

In some exemplary embodiments, the target RNA molecules comprise one or more miRNA molecules selected from the group consisting of ‘hsa-miR-576-5p, hsa-miR-539-5p, hsa-miR-500a-5p, hsa-miR-376c-3p, hsa-miR-339-3p, hsa-miR-185-3p, hsa-miR-17-3p, and hsa-miR-101-3p.

In some exemplary embodiments, the target RNA molecules comprise one or more miRNA molecules selected from the group consisting of hsa-miR-17-3p, hsa-miR-500a-5p, hsa-miR-215-5p, hsa-miR-1271-5p, and hsa-miR-151a-5p.

In some exemplary embodiments, the target RNA molecules comprise one or more miRNA molecules selected from the group consisting of hsa-miR-17-3p, hsa-miR-500a-5p, and hsa-miR-151a-5p.

In some exemplary embodiments, the target RNA molecules comprise one or more miRNA molecules selected from the group consisting of cel-miR-39-3p, hsa-Let-7a-5p, hsa-Let-7d-3p, hsa-Let-7i-5p, hsa-miR-1224-5p, hsa-miR-125b-5p, hsa-miR-126-3p, hsa-miR-1281, hsa-miR-130a-3p, hsa-mir-135a1-5p, hsa-miR-142-3p, hsa-miR-145-3p, hsa-miR-145-5p, hsa-miR-146a-5p, hsa-miR-155-5p, hsa-miR-15a-5p, hsa-miR-15b-5p, hsa-miR-16-5p, hsa-miR-17-3p, hsa-miR-1825, hsa-miR-186-5p, hsa-miR-18a-5p, hsa-miR-18b-3p, hsa-miR-191-5p, hsa-miR-195-5p, hsa-miR-199a-1-3p, hsa-miR-200b-3p, hsa-miR-203a-3p, hsa-miR-204-5p, hsa-miR-208a-3p, hsa-miR-21-5p, hsa-miR-210-3p, hsa-miR-211-5p, hsa-miR-215-5p, hsa-miR-216a-5p, hsa-miR-223-3p, hsa-miR-23a-3p, hsa-miR-24-3p, hsa-miR-299-5p, hsa-miR-30a-3p, hsa-miR-30c-5p, hsa-miR-30d-5p, hsa-miR-320a-3p, hsa-miR-323a-3p, hsa-miR-29b-3p, hsa-miR-3615, hsa-miR-377-3p, hsa-miR-378a-3p, hsa-miR-378h, hsa-miR-382-5p, hsa-miR-411-5p, hsa-miR-423-5p, hsa-miR-4286, hsa-miR-449b-5p, hsa-miR-449c-5p, hsa-miR-451a, hsa-miR-484, hsa-miR-487a-5p, hsa-miR-494-3p, hsa-miR-499a-5p, hsa-miR-500a-3p, hsa-miR-625-5p, hsa-miR-877-5p, hsa-miR-92b-3p, hsa-miR-93-5p, hsa-mir19a-5p, hsa-mir208a-3p, hsa-miR-101-3p, hsa-miR-136-3p, hsa-miR-192-3p, hsa-miR-194-5p, hsa-miR-196b-5p, hsa-miR-214-3p, hsa-miR-339-3p, hsa-miR-4746-5p, hsa-miR-500a-5p, hsa-miR-539-5p, hsa-miR-576-5p, hsa-miR-1-3p, hsa-miR-1277-5p, hsa-miR-139-5p, hsa-miR-146b-5p, hsa-miR-183-5p, hsa-miR-188-5p, hsa-miR-190a-5p, hsa-miR-200a-3p, hsa-miR-205-5p, hsa-miR-2115-3p, hsa-miR-3690, hsa-miR-376a-3p, hsa-miR-376b-3p, hsa-miR-412-5p, hsa-miR-449a, hsa-miR-539-3p, hsa-miR-551a, hsa-miR-582-3p, hsa-miR-628-5p, hsa-miR-629-5p, hsa-miR-642a-5p, hsa-miR-651-5p, hsa-miR-873-5p, hsa-miR-887-3p, hsa-miR-376c-3p, hsa-miR-92b-5p, hsa-miR-6734-5p, hsa-miR-664a-5p, hsa-miR-4488, hsa-miR-381-3p, hsa-miR-1271-5p, hsa-miR-101, hsa-miR-99b-5p, hsa-miR-660-3p, hsa-miR-329-3p, hsa-miR-410-3p, hsa-miR-185-3p, hsa-miR-569, hsa-miR-454-5p, hsa-miR-3912-3p, hsa-miR-363-3p, hsa-miR-27a-5p, hsa-miR-191-5p, hsa-miR-145-5p, and hsa-let-7i-3p, ‘hsa-miR-652-3p’, ‘hsa-miR-584-5p, hsa-miR-378a-3p, hsa-miR-338-3p, hsa-miR-29c-3p, hsa-miR-221-3p, hsa-miR-20a-5p, hsa-miR-199b-3p, hsa-miR-181a-5p, hsa-miR-17-5p, hsa-miR-151a-3p, hsa-miR-148a-3p, hsa-miR-143-3p, and hsa-miR-151a-5p.

In one aspect, the present disclosure relates to a method of preparing a composition of amplified nucleic acids derived from a sample obtained from a transplant recipient useful for determining transplant rejection, comprising:
extracting fragmented or intact RNA (such as mRNA) derived from the sample obtained from the transplant recipient, wherein the extracted RNA comprises donor- and/or recipient-derived RNA;
preparing a composition of nucleic acids from the RNA by performing reverse transcription of the RNA to produce complementary DNA (cDNA) followed by universal amplification and/or targeted multiplex amplification of 10-50,000 target loci in a single reaction volume;
measuring the amount of RNA in the sample based on the amplicons obtained in step b) to quantify the amount of donor-derived RNA; and
determining whether the amount of donor derived RNA or a function thereof exceeds a cutoff threshold indicating transplant rejection.

In some exemplary embodiments, the methods disclosed herein further comprise utilizing CRISPR-Cas to target and deplete contaminating or overabundant nucleic acid species in the sample, thereby increasing the fraction of desired reads that map to a target loci of interest per sample and the sample throughput per sequencing run.

In some exemplary embodiments, the sample comprises whole blood or hemolysis tainted blood, serum or plasma samples, and wherein a plurality of guide RNAs are used to target a plurality of loci in the same reaction to deplete contaminating or overabundant nucleic acid, thereby increasing a detection rate of the target loci.

In some exemplary embodiments, the contaminating or overabundant nucleic acid species comprise hemoglobin mRNA, tRNA, and rRNA, and miR-451, miR-144, and miR-486.

In some exemplary embodiments, the methods disclosed herein further comprise depleting adaptor dimers, primer dimers, unwanted ligation products from the composition of amplified nucleic acids comprising target loci, thereby increasing the fraction of desired reads that map to a target loci of interest per sample and the sample throughput per sequencing run. In some exemplary embodiments, Cas9/Cas12a is utilized to remove nucleic acid species after reverse transcription of RNA and before multiplex amplification. In some exemplary embodiments, Cas9/Cas 12a is utilized to remove nucleic acid species after 1-10 cycles of multiplex amplification of the complementary DNA.

In some exemplary embodiments, the contaminating or overabundant nucleic acid species are RNA, and wherein Cas13 is used to remove the contaminating or overabundant RNA species from the sample. In some exemplary embodiments, the methods disclosed herein further comprise:
measuring the amount of donor-derived cell-free DNA in a sample obtained from the transplant recipient, extracting cell-free DNA from the sample obtained from the transplant recipient, wherein the extracted cell-free DNA comprises donor-derived cell-free DNA and recipient-derived cell-free DNA; performing targeted amplification of the extracted DNA at 50-50,000 target loci in a single reaction volume; sequencing the amplified DNA by high-throughput sequencing to obtain sequencing reads and quantifying the amount of donor-derived cell-free DNA based on the sequencing reads, determining transplant rejection based on whether the amount of donor-derived cell-free DNA or a function thereof exceeds a cutoff threshold of cell-free DNA amount that indicates transplant rejection, wherein transplant rejection is determined based on whether both the amount of donor-derived RNA and the amount of donor derived cell-free DNA or function thereof exceeds a cutoff threshold that indicates transplant rejection.

In some exemplary embodiments, the amount of donor-derived mRNA is determined by using ratiometric and/or machine learning-artificial intelligence comparisons at a single or a plurality of time points.

In some exemplary embodiments, the sample comprises blood, plasma, serum, CSF or urine.

In some exemplary embodiments, the transplant recipient is a human subject.

In some exemplary embodiments, the transplant recipient has received a plurality of transplanted organs selected from pancreas, kidney, liver, lung, heart, intestinal, thymus or uterus.

In some exemplary embodiments, the one or more transplanted organs are from the same transplant donors.

In some exemplary embodiments, the one or more transplanted organs are from multiple different transplant donors.

In some exemplary embodiments, the transplant recipient has received simultaneous transplantation of more than one organ.

In some exemplary embodiments, the RNA biomarkers indicate an increased immune response, or a decreased immune response.

In some exemplary embodiments, the methods disclosed herein further comprise measuring the amounts of RNA longitudinally for the same transplant recipient; determining a longitudinal change in the amount of RNA.

In some exemplary embodiments, the RNA is cell-free RNA.

In some exemplary embodiments, the cell-free RNA is derived from exosomes or microvesicles.

In some exemplary embodiments, the RNA is small messenger RNA (mRNA).

In some exemplary embodiments, the RNA is small noncoding RNA (sncRNA).

In some exemplary embodiments, the sncRNA comprises micro RNA (miRNA), piwi-interacting RNA (piRNA), small nucleolar RNA (snoRNA), small nuclear RNA (snRNA), or miscellaneous RNA (miscRNA).

In some exemplary embodiments, step (b) comprises multiplex amplification of at least 100, at least 500, at least 1000, at least 2000 target loci, from 10-1000, 100-10000, 50-50000, or 500-20000 target loci, using at least 100, at least 500, at least 1000, at least 2000, from 10-1000, 100-10000, 50-50000, 500-20000 pairs of forward and reverse PCR primers.

In some exemplary embodiments, the amount of RNA or cell-free DNA is measured by a quantitative PCR method, and wherein the quantitative PCR method comprises real-time PCR or digital PCR.

In some exemplary embodiments, the amount of RNA or cell-free DNA is measured by massively multiplex PCR (mmPCR) to obtain amplicons comprising biomarkers, and sequencing of the amplicons.

In some exemplary embodiments, the amount of RNA or cell-free DNA is measured by using microarray.

In some exemplary embodiments, the amount of RNA or cell-free DNA is measured by using molecular barcodes and microscopic imaging (such as NanoString nCounter®).

In some exemplary embodiments, the cutoff threshold is an estimated percentage of donor-derived RNA out of total RNA or a function thereof.

In some exemplary embodiments, the cutoff threshold is an estimated percentage of donor-derived cell-free DNA out of total cell-free DNA or a functional thereof.

In some exemplary embodiments, the cutoff threshold is proportional to an absolute donor-derived RNA or cell-free DNA concentration.

In some exemplary embodiments, a rejection risk for the transplant recipient can be determined based on the amount of donor-derived RNA and/or amount of cell-free DNA.

In some exemplary embodiments, the rejection risk for the transplant recipient is determined using machine learning such as Mann-Whitney test, Benjamini-Hochberg (BH) Procedure, logistic LASSO regression, Recursive Feature Elimination (RFE)—support vector machine (SVM), RFE-Random Forest, or gradient boosting.

In some exemplary embodiments, the machine learning is the logistic LASSO regression, or random forest analysis, and further incorporates one or more parameters selected from time post-transplantation, age of transplant recipient and/or transplant donor, gender of transplant recipient and/or transplant donor, the amount of RNA in the sample, or the amount of total cell-free DNA in the sample or a function thereof.

In some exemplary embodiments, the sample is obtained from the transplant recipient less than 18 months post-transplantation.

### BRIEF DESCRIPTION OF THE DRAWINGS

FIG. 1. Cas9 complex diagram from Integrated DNA Technologies (IDT). The spacer in the gRNA guides the Cas9 complex to the protospacer sequence in the target DNA. In the presence of a PAM sequence in the target DNA, both strands of the target will be cleaved to leave a blunt end.

FIG. 2. Removal of unwanted sequences from sequencing libraries. When an unwanted sequence is ligated to the adaptor, a Cas9-gRNA complex complementary to this unwanted sequence will be introduced. This will result in removal of the adaptor, preventing PCR amplification. This step should be performed on double-stranded reverse transcribed DNA before and during library preparation.

FIG. 3. Significance Distribution of Heart miRNA. The Graph depicts Frequency Ratio vs specific miRNA. High ranking miRNA candidates appear similarly significant between Frequency ratio and Frequency Ratio simulated methods.

FIG. 4. Significance Distribution of Heart miRNA. The graph depicts Bayes value vs specific miRNA. High ranking miRNA candidates appear similarly significant between Bayes and Bayes Ratio simulated methods.

FIG. 5. Significance Distribution of kidney miRNA. The graph depicts Frequency Ratio vs specific miRNA. High ranking miRNA candidates appear similarly significant between Frequency Ratio & Frequency Ratio simulated methods.

FIG. 6. Significance Distribution of Kidney miRNA. The graph depicts Bayes value vs specific miRNA. High ranking miRNA candidates appear similarly significant between Bayes and Bayes Ratio simulated methods.

FIG. 7. Significance Distribution of Immune miRNA. The graph depicts Frequency Ratio vs specific miRNA. High ranking miRNA candidates appear similarly significant between Frequency ratio and Frequency Ratio simulated methods.

FIG. 8. Significance Distribution of Immune miRNA. The graph depicts Bayes value vs specific miRNA. High ranking miRNA candidates appear similarly significant between Bayes & Bayes Ratio simulated methods.

FIG. 9. Significance Distribution of liver miRNA. The graph depicts Significance Concordance Comparison between Frequency ratio and simulated ratio. High ranking miRNA candidates appear similarly significant between Frequency ratio methods.

FIG. 10. Significance Distribution of Lung miRNA. The graph depicts Significance Concordance Comparison between Bayes and simulated Bayes. High ranking miRNA candidates appear similarly significant between Bayes methods.

FIG. 11. Significance Distribution of lung miRNA. The graph depicts Frequency Ratio calculations. High ranking miRNA candidates appear similarly significant between Frequency ratio and Frequency Ratio simulated methods.

FIG. 12. Significance Distribution of Lung miRNA. The graph depicts Significance Concordance Comparison between Bayes and simulated Bayes. High ranking miRNA candidates appear similarly significant between Bayes methods.

FIG. 13. The diagram depicts the highest specificity miRNA in the 14q32.31 miRNA cluster. This cluster is regulated by CH3 genomic imprinting and miRNA expression does not differ between men and women, and has no correlation with age.

FIG. 14. Predicted miRNA targets from RefSeq mRNA. 80% of 40,185 mRNA splice variants have more than 20 predicted microRNA targets. The median predicted microRNA targets is 61 miRNA.

### DETAILED DESCRIPTION

In at least one aspect, the present disclosure relates to a method of preparing a composition of amplified nucleic acids derived from a sample obtained from a transplant recipient useful for determining transplant rejection, comprising: extracting fragmented or intact RNA (such as mRNA) derived from sample of the transplant recipient, wherein the extracted RNA comprises donor- and/or recipient-derived RNA; preparing a composition of nucleic acids from the RNA by performing reverse transcription of the RNA to produce complementary DNA (cDNA) followed by targeted amplification to obtain amplicons of a plurality of target RNA molecules indicative of transplant rejection or organ health. In some exemplary embodiments of the present disclosure, the methods further comprise measuring the amount of the target RNA molecules in the sample based on the amplicons obtained in step b) to quantify the amount of donor-derived target RNA molecules; and determining whether the amount of donor derived target RNA molecules or a function thereof exceeds a cutoff threshold indicating transplant rejection.

In one aspect, the present disclosure relates to a method of preparing a composition of amplified nucleic acids derived from a sample obtained from a transplant recipient useful for determining transplant rejection, wherein the sample comprises donor- and/or recipient-derived RNA;
a) preparing a composition of nucleic acids from the RNA by performing reverse transcription of the RNA to produce complementary DNA (cDNA) followed by amplification to obtain amplicons;
b) measuring the amount of RNA in the sample based on the amplicons obtained in step (b) to quantify the amount of donor-derived RNA and/or the amount of a plurality of target RNA molecules indicative of transplant rejection or organ health; and
c) determining transplant rejection or organ health based on (i) whether the amount of donor derived RNA or a function thereof exceeds a cutoff threshold indicating transplant rejection, and (ii) whether the amount of a plurality of target RNA molecules indicative of transplant rejection or organ health or a function thereof exceeds a cutoff threshold indicating transplant rejection.

In at least one aspect, the present invention relates to a method of preparing a composition of amplified nucleic acids derived from a sample obtained from a transplant recipient useful for determining transplant rejection, comprising: extracting fragmented or intact RNA (such as mRNA) derived from sample of the transplant recipient, wherein the extracted RNA comprises donor- and/or recipient-derived RNA; preparing a composition of nucleic acids from the RNA by performing reverse transcription of the RNA to produce complementary DNA (cDNA) followed by targeted multiplex amplification of 10-50,000 target loci in a single reaction volume; measuring the amount of RNA in the sample based on the amplicons obtained in step b) to quantify the amount of donor-derived RNA; and determining whether the amount of donor derived RNA or a function thereof exceeds a cutoff threshold indicating transplant rejection. In some exemplary embodiments, the term “amount of donor-derived RNA” is equivalent with “total amount of donor-derived RNA.”

In some exemplary embodiments, the RNA comprises mRNA. In some exemplary embodiments, the RNA comprises microRNA. In some exemplary embodiments, the RNA comprises biomarkers useful for determining or monitoring a disease or condition. In some exemplary embodiments, the disease or condition may be transplant rejection. In some exemplary embodiments, the biomarkers indicate an immune response. In some exemplary embodiments, the biomarker is a gene or polymorphism associated with a disease or condition such as transplant rejection or cancer as further described herein. In some exemplary embodiments, the donor organ is heart and the biomarker comprises hsa-miR-377-3p, hsa-miR-30c-5p, hsa-miR-30d-5p, hsa-miR-195-5p, hsa-miR-208a, hsa-miR-499a-5p, and/or hsa-miR-186-5p.

In some exemplary embodiments, the donor organ is liver and the biomarker comprises hsa-miR-15b-5p, hsa-miR-18b-3p, and/or hsa-miR-223.

In some exemplary embodiments, the donor organ is lung and the biomarker comprises hsa-miR-16-5p.

In some exemplary embodiments, the donor organ is kidney and the biomarker comprises hsa-miR-423-5p, and/or hsa-miR-216a-5p.

In one aspect, the present disclosure relates to a method of preparing a composition of amplified nucleic acids derived from a sample obtained from a transplant recipient useful for determining transplant rejection, comprising: a) extracting fragmented or intact mRNA derived from sample of the transplant recipient, wherein the extracted mRNA comprises donor- and/or recipient-derived mRNA; b) preparing a composition of nucleic acids from the mRNA by performing reverse transcription and targeted multiplex amplification of 10-50,000 target loci in a single reaction volume; c) measuring the amount of mRNA in the sample based on the amplicons obtained in step b) to quantify the amount of donor-derived mRNA; and d) determining whether the amount of donor derived mRNA or a function thereof exceeds a cutoff threshold indicating transplant rejection.

In another aspect, the present disclosure relates to a method of method of preparing a composition of amplified nucleic acids derived from a sample obtained from a transplant recipient useful for determination of transplant rejection, comprising: (a) extracting RNA comprising small non-coding RNA (sncRNA) from the sample obtained from the transplant recipient, wherein the extracted RNA comprises donor-derived sncRNA and recipient-derived sncRNA; (b) preparing a composition of nucleic acids from the RNA by reverse transcription and amplification, and sequencing the amplified nucleic acids by high-throughput sequencing to obtain sequencing reads; (c) measuring the amount of sncRNA comprising a plurality of biomarker indicative of transplant rejection based on the sequencing reads in the sample obtained from the transplant recipient; and (d) determining whether the amount of recipient-derived sncRNA comprising biomarkers indicative of transplant rejection exceeds a cutoff threshold or a function thereof.

### Samples Comprising Nucleic Acids and Methods for Obtaining Samples and Extracting Nucleic Acids

The method disclosed herein comprises extracting fragmented or intact RNA derived from a sample obtained from a subject. The subject may be a transplant recipient, or a subject suffering from any disease or disorder described herein. In some exemplary embodiments, the transplant recipient is a human subject. In some exemplary embodiments, the transplant recipient has received a plurality of transplanted organs selected from pancreas, kidney, liver, lung, heart, intestinal, thymus or uterus. In some exemplary embodiments, the one or more transplanted organs are from the same transplant donors. In some exemplary embodiments, the one or more transplanted organs are from more multiple different transplant donors. In some exemplary embodiments, the transplant recipient has received simultaneous transplantation of more than one organ.

In some exemplary embodiments, the transplant recipient has received one or more transplanted organs selected from kidney, liver, heart, lung, pancreas, intestinal, thymus, and uterus. In some exemplary embodiments, the transplant recipient has received a kidney transplant. In some exemplary embodiments, the transplant recipient has received a liver transplant. In some exemplary embodiments, the transplant recipient has received a heart transplant. In some exemplary embodiments, the transplant recipient has received a lung transplant. In some exemplary embodiments, the transplant recipient has received a pancreas transplant. In some exemplary embodiments, the transplant recipient has received an intestinal transplant. In some exemplary embodiments, the transplant recipient has received a thymus transplant. In some exemplary embodiments, the transplant recipient has received a uterus transplant.

In some exemplary embodiments, the sample is obtained from the transplant recipient less than 18 months post-transplantation, less than 17 months post-transplantation, less than 16 months post-transplantation, less than 15 months post-transplantation, less than 14 months post-transplantation, less than 13 months post-transplantation, or less than 12 months post-transplantation. In some exemplary embodiments, the sample is obtained from the transplant recipient between 0 and 2 months post-transplantation, between 2 and 4 months post-transplantation, between 4 and 6 months post-transplantation, between 6 and 9 months post-transplantation, between 9 and 12 months post-transplantation, or between 12 and 18 months post-transplantation.

In some exemplary embodiments, the methods disclosed herein further comprise measuring the amounts of RNA longitudinally for the same transplant recipient; determining a longitudinal change in the amount of RNA. In some exemplary embodiments, the amounts of RNA is the total amount of RNA derived from the donor organ.

In some exemplary embodiments, the transplant recipient has received one or more organs from the same transplant donors. In some exemplary embodiments, the transplant recipient has received one or more organs from more multiple different transplant donors. In some exemplary embodiments, the transplant recipient has received simultaneous transplantation of more than one organ.

The samples may be a body fluid sample, a tissue, a organ, or individual cells. In some exemplary embodiments the sample comprises blood, plasma, serum, CSF or urine. In some exemplary embodiments, the sample is blood. In some exemplary embodiments, the sample is blood, plasma, or serum.

In some exemplary embodiments, the subject is suffering from a disease or disorder. In some exemplary embodiments, the disease or the disease status comprises a cancer, recurrence or metastasis of the cancer, an immune disease or disorder, pre-eclampsia, or congenital heart disease (CHD). In some exemplary embodiments, the cancer is breast cancer, lung cancer, liver cancer, skin cancer, prostate cancer, bladder cancer, or colorectal cancer.

### Nucleic Acids and Methods of Extracting or Enriching Nucleic Acids

The methods disclosed herein comprises extracting nucleic acids from a sample derived from a subject. The nucleic acids may be cell-free DNA, cellular DNA, DNA extracted from exosomes, cell-free RNA, cellular RNA, or RNA extracted from exosomes. The term “RNA” refers herein to any type of RNA, including messenger RNA (mRNA) or small non-coding RNA (sncRNA) such as micro RNA (miRNA). In some exemplary embodiments, the RNA is messenger RNA (mRNA) such as cell-free, cellular, or exosome RNA. In some exemplary embodiments, the RNA comprises small non-coding RNA (sncRNA). In some exemplary embodiments, the sncRNA comprises micro RNA (miRNA), piwi-interacting RNA (piRNA), small nucleolar RNA (snoRNA), small nuclear RNA (snRNA), or miscellaneous RNA (miscRNA). In some exemplary embodiments, the cell-free sncRNA is derived from exosomes or microvesicles.

In some exemplary embodiments, nucleic acids are extracted by using size exclusion. In some exemplary embodiments, cell-free DNA or RNA is isolated from cellular DNA or RNA based on size. In some exemplary embodiments, nucleic acids are isolated by using affinity chromatography.

In some exemplary embodiments, nucleic acids are preferentially enriched. Nucleic acids may be preferentially enriched by using preferential enrichment at a locus or target site. Such preferential enrichment refers to any method that results in the percentage of molecules of nucleic acids in a post-enrichment nucleic acid mixture that correspond to the locus being higher than the percentage of molecules of nucleic acids in the pre-enrichment nucleic acid mixture that correspond to the locus. The method may involve selective amplification of nucleic acid molecules that correspond to a locus. The method may involve removing nucleic acid molecules that do not correspond to the locus. The method may involve a combination of methods. The degree of enrichment is defined as the percentage of molecules of nucleic acids in the post-enrichment mixture that correspond to the locus or target divided by the percentage of molecules of nucleic acids in the pre-enrichment mixture that correspond to the locus or target. Preferential enrichment may be carried out at a plurality of loci. In some exemplary embodiments of the present disclosure, the degree of enrichment is greater than 20. In some exemplary embodiments of the present disclosure, the degree of enrichment is greater than 200. In some exemplary embodiments of the present disclosure, the degree of enrichment is greater than 2,000. When preferential enrichment is carried out at a plurality of loci, the degree of enrichment may refer to the average degree of enrichment of all of the loci in the set of loci.

Amplification refers to a method that increases the number of copies of nucleic acid molecules. Selective Amplification may refer to a method that increases the number of copies of a particular nucleic acid molecules, or nucleic acid molecules that correspond to a particular region of nucleic acid molecules. It may also refer to a method that increases the number of copies of a particular targeted molecule of nucleic acid molecules, or targeted region of nucleic acid molecules more than it increases non-targeted molecules or regions of nucleic acid molecules.

Selective amplification may be a method of preferential enrichment. Universal Priming Sequence refers to a DNA sequence that may be appended to a population of target DNA molecules, for example by ligation, PCR, or ligation mediated PCR. Once added to the population of target molecules, primers specific to the universal priming sequences can be used to amplify the target population using a single pair of amplification primers. Universal priming sequences are typically not related to the target sequences. Universal Adapters, or ‘ligation adaptors’ or ‘library tags’ are DNA molecules containing a universal priming sequence that can be covalently linked to the 5-prime and 3-prime end of a population of target double stranded DNA molecules. The addition of the adapters provides universal priming sequences to the 5-prime and 3-prime end of the target population from which PCR amplification can take place, amplifying all molecules from the target population, using a single pair of amplification primers. Targeting refers to a method used to selectively amplify or otherwise preferentially enrich those molecules of DNA that correspond to a set of loci, in a mixture of DNA.

Particular nucleic acids may also be enriched for by using hybrid capture. In some exemplary embodiments, preferentially enriching the RNA at the plurality of biomarkers comprises: obtaining a set of hybrid capture probes; hybridizing the hybrid capture probes to the RNA in the sample; and physically separating the hybridized RNA from the sample of RNA from the unhybridized RNA from the sample. In some exemplary embodiments, preferentially enriching the sncRNA such as miRNA at the plurality of biomarkers comprises: obtaining a set of hybrid capture probes; hybridizing the hybrid capture probes to the miRNA in the sample; and physically separating the hybridized miRNA from the sample of RNA from the unhybridized RNA from the sample. In some exemplary embodiments, preferentially enriching preselected mRNA comprises: obtaining a set of hybrid capture probes; hybridizing the hybrid capture probes to the mRNA in the sample; and physically separating the hybridized mRNA from the sample of RNA from the unhybridized RNA from the sample.

In some exemplary embodiments, in the methods disclosed herein, the DNA is preferentially enriched at the target loci or a biomarker.

Biomarker″ refers to a molecule that is an indicator of an abnormal biological condition (e.g., disease or disorder, or transplant rejection). For example, a biomarker may be a gene product that (a) is expressed at higher or lower levels, (b) has an altered ratio relative to another biomarker, (c) is present at higher or lower levels, (d) is a variant or mutant of the gene product, or (e) is simply present or absent, in a cell or tissue sample from a subject having or suspected of having a disease as compared to an undiseased tissue or cell sample from the subject having or suspected of having a disease, or as compared to a cell or tissue sample from a subject or a pool of subjects not having or suspected of having the disease. In the context of transplant, the biomarker may be indicative of poor donor organ health or transplant rejection. That is, one or more gene products are sufficiently specific to the test sample that one or more may be used to identify, predict, or detect the presence of transplant rejection, disease, risk of disease, risk of a given event or change in disease status, or provide information for a proper or improved therapeutic regimen.

In some exemplary embodiments, one or more biomarkers can be one or a collection of genetic aberrations, which is used herein to refer to the nucleic acid amounts as well as nucleic acid variants within the nucleic acid-containing particles. Specifically, genetic aberrations include, without limitation, over-expression of a gene (e.g., an oncogene) or a panel of genes, under-expression of a gene (e.g., a tumor suppressor gene such as p53 or RB) or a panel of genes, alternative production of splice variants of a gene or a panel of genes, gene copy number variants (CNV) (e.g., DNA double minutes), nucleic acid modifications (e.g., methylation, acetylation and phosphorylation), single nucleotide polymorphisms (SNPs), chromosomal rearrangements (e.g., inversions, deletions and duplications), and mutations (insertions, deletions, duplications, missense, nonsense, synonymous or any other nucleotide changes) of a gene or a panel of genes, which mutations, in many cases, ultimately affect the activity and function of the gene products, lead to alternative transcriptional splice variants and/or changes of gene expression level, or combinations of any of the foregoing.

In some exemplary embodiments, preferentially enriching the DNA in the sample at the plurality of polymorphic loci includes obtaining a plurality of pre-circularized probes where each probe targets one of the polymorphic loci, and where the 3′ and 5′ end of the probes are designed to hybridize to a region of DNA that is separated from the polymorphic site of the locus by a small number of bases, where the small number is 1, 2, 3, 4, 5, 6, 7, 8, 9, 10, 11, 12, 13, 14, 15, 16, 17, 18, 19, 20, 21 to 25, 26 to 30, 31 to 60, or a combination thereof, hybridizing the pre-circularized probes to DNA from the sample, filling the gap between the hybridized probe ends using DNA polymerase, circularizing the pre-circularized probe, and amplifying the circularized probe.

In some exemplary embodiments, preferentially enriching the DNA at the plurality of polymorphic loci includes obtaining a plurality of ligation-mediated PCR probes where each PCR probe targets one of the polymorphic loci, and where the upstream and downstream PCR probes are designed to hybridize to a region of DNA, on one strand of DNA, that is separated from the polymorphic site of the locus by a small number of bases, where the small number is 1, 2, 3, 4, 5, 6, 7, 8, 9, 10, 11, 12, 13, 14, 15, 16, 17, 18, 19, 20, 21 to 25, 26 to 30, 31 to 60, or a combination thereof, hybridizing the ligation-mediated PCR probes to the DNA from the first sample, filling the gap between the ligation-mediated PCR probe ends using DNA polymerase, ligating the ligation-mediated PCR probes, and amplifying the ligated ligation-mediated PCR probes.

In some exemplary embodiments, preferentially enriching the DNA at the plurality of polymorphic loci includes obtaining a plurality of hybrid capture probes that target the polymorphic loci, hybridizing the hybrid capture probes to the DNA in the sample and physically removing some or all of the unhybridized DNA from the first sample of DNA.

In some exemplary embodiments, the hybrid capture probes are designed to hybridize to a region that is flanking but not overlapping the polymorphic site. In some exemplary embodiments, the hybrid capture probes are designed to hybridize to a region that is flanking but not overlapping the polymorphic site, and where the length of the flanking capture probe may be selected from the group consisting of less than about 120 bases, less than about 110 bases, less than about 100 bases, less than about 90 bases, less than about 80 bases, less than about 70 bases, less than about 60 bases, less than about 50 bases, less than about 40 bases, less than about 30 bases, and less than about 25 bases. In some exemplary embodiments, the hybrid capture probes are designed to hybridize to a region that overlaps the polymorphic site, and where the plurality of hybrid capture probes comprise at least two hybrid capture probes for each polymorphic loci, and where each hybrid capture probe is designed to be complementary to a different allele at that polymorphic locus.

In some exemplary embodiments, preferentially enriching the DNA at a plurality of polymorphic loci includes obtaining a plurality of inner forward primers where each primer targets one of the polymorphic loci, and where the 3′ end of the inner forward primers are designed to hybridize to a region of DNA upstream from the polymorphic site, and separated from the polymorphic site by a small number of bases, where the small number is selected from the group consisting of 1, 2, 3, 4, 5, 6 to 10, 11 to 15, 16 to 20, 21 to 25, 26 to 30, or 31 to 60 base pairs, optionally obtaining a plurality of inner reverse primers where each primer targets one of the polymorphic loci, and where the 3′ end of the inner reverse primers are designed to hybridize to a region of DNA upstream from the polymorphic site, and separated from the polymorphic site by a small number of bases, where the small number is selected from the group consisting of 1, 2, 3, 4, 5, 6 to 10, 11 to 15, 16 to 20, 21 to 25, 26 to 30, or 31 to 60 base pairs, hybridizing the inner primers to the DNA, and amplifying the DNA using the polymerase chain reaction to form amplicons.

In some exemplary embodiments, the method also includes obtaining a plurality of outer forward primers where each primer targets one of the polymorphic loci, and where the outer forward primers are designed to hybridize to the region of DNA upstream from the inner forward primer, optionally obtaining a plurality of outer reverse primers where each primer targets one of the polymorphic loci, and where the outer reverse primers are designed to hybridize to the region of DNA immediately downstream from the inner reverse primer, hybridizing the first primers to the DNA, and amplifying the DNA using the polymerase chain reaction.

In some exemplary embodiments, the method also includes obtaining a plurality of outer reverse primers where each primer targets one of the polymorphic loci, and where the outer reverse primers are designed to hybridize to the region of DNA immediately downstream from the inner reverse primer, optionally obtaining a plurality of outer forward primers where each primer targets one of the polymorphic loci, and where the outer forward primers are designed to hybridize to the region of DNA upstream from the inner forward primer, hybridizing the first primers to the DNA, and amplifying the DNA using the polymerase chain reaction.

In some exemplary embodiments, preparing the first sample further includes appending universal adapters to the DNA in the first sample and amplifying the DNA in the first sample using the polymerase chain reaction. In some exemplary embodiments, at least a fraction of the amplicons that are amplified are less than 100 bp, less than 90 bp, less than 80 bp, less than 70 bp, less than 65 bp, less than 60 bp, less than 55 bp, less than 50 bp, or less than 45 bp, and where the fraction is 10%, 20%, 30%, 40%, 50%, 60%, 70%, 80%, 90%, or 99%.

In some exemplary embodiments, amplifying the DNA is done in one or a plurality of individual reaction volumes, and where each individual reaction volume contains more than 100 different forward and reverse primer pairs, more than 200 different forward and reverse primer pairs, more than 500 different forward and reverse primer pairs, more than 1,000 different forward and reverse primer pairs, more than 2,000 different forward and reverse primer pairs, more than 5,000 different forward and reverse primer pairs, more than 10,000 different forward and reverse primer pairs, more than 20,000 different forward and reverse primer pairs, more than 50,000 different forward and reverse primer pairs, or more than 100,000 different forward and reverse primer pairs.

In some exemplary embodiments, preparing the sample further comprises dividing the sample into a plurality of portions, and where the DNA in each portion is preferentially enriched at a subset of the plurality of polymorphic loci. In some exemplary embodiments, the inner primers are selected by identifying primer pairs likely to form undesired primer duplexes and removing from the plurality of primers at least one of the pair of primers identified as being likely to form undesired primer duplexes. In some exemplary embodiments, the inner primers contain a region that is designed to hybridize either upstream or downstream of the targeted polymorphic locus, and optionally contain a universal priming sequence designed to allow PCR amplification. In some exemplary embodiments, at least some of the primers additionally contain a random region that differs for each individual primer molecule. In some exemplary embodiments, at least some of the primers additionally contain a molecular barcode.

In some exemplary embodiments, the method comprises: (a) performing multiplex polymerase chain reaction (PCR) on a nucleic acid sample comprising target loci to simultaneously amplify at least 1,000 distinct target loci using either (i) at least 1,000 different primer pairs, or (ii) at least 1,000 target-specific primers and a universal or tag-specific primer, in a single reaction volume to produce amplified products comprising target amplicons; and (b) sequencing the amplified products. In some exemplary embodiments, the method does not comprise using a microarray.

In some exemplary embodiments, the method comprises (a) performing multiplex polymerase chain reaction (PCR) on the cell free DNA sample comprising target loci to simultaneously amplify at least 1,000 distinct target loci using either (i) at least 1,000 different primer pairs, or (ii) at least 1,000 target-specific primers and a universal or tag-specific primer, in a single reaction volume to produce amplified products comprising target amplicons; and b) sequencing the amplified products. In some exemplary embodiments, the method does not comprise using a microarray.

In some exemplary embodiments, mRNA is isolated by using probes that hybridize to the poly-A tail of the mRNA molecules.

After blood draw and before nucleic acid extraction, blood cells within a blood sample may burse and shed long fragments of DNA into the sample, which would increase the total amount of cell-free DNA (cfDNA) and background noise, distorting thd % of dd-cfDNA detected. In order to reduce such background noise, and based on the observation that dd-cfDNA is typically shorter than DNA shredded from a transplant recipient blood cell, two particular enrichments for dd-cfDNA are contemplated. In one embodiment, a size selection is applied to select for shorter cfDNA. In another embodiment, a universal amplification step is applied to reduce noise (e.g., before applying multiplex PCR), based on the hypothesis that shorter dd-cfDNA (often in mononucleosome form) is amplified more efficiently than longer transplant recipient-derived DNA.

### Target Genes and Loci

The nucleic acids may comprise biomarkers indicative of an immune response, or various diseases or conditions as described elsewhere herein.

In some exemplary embodiments, the method comprises extracting fragmented or intact mRNA derived from sample of the transplant recipient, wherein the extracted mRNA comprises donor- and/or recipient-derived mRNA, and wherein the mRNA comprises a plurality of biomarkers indicative of an immune response, or a disease or disorder. In some exemplary embodiments, the biomarker indicates an increased immune response. In some exemplary embodiments, the biomarker indicates a decreased immune response. In some particular embodiments, sncRNA (such as miRNA) biomarkers indicate an increased immune response, or a decreased immune response.

In some exemplary embodiments, the presently disclosed method comprises pre-selecting RNA target molecules. In some exemplary embodiments, the RNA target molecules comprises RNA species known to be relevant for assessing organ health. In some exemplary embodiments, the present disclosure provides methods for identifying RNA target molecules that are relevant for assessing organ health.

In some exemplary embodiments, the target RNA molecules comprise one or more miRNA molecules selected from the group consisting of cel-miR-39-3p, hsa-Let-7a-5p, hsa-Let-7d-3p, hsa-Let-7i-5p, hsa-miR-1224-5p, hsa-miR-125b-5p, hsa-miR-126-3p, hsa-miR-1281, hsa-miR-130a-3p, hsa-mir-135a1-5p, hsa-miR-142-3p, hsa-miR-145-3p, hsa-miR-145-5p, hsa-miR-146a-5p, hsa-miR-155-5p, hsa-miR-15a-5p, hsa-miR-15b-5p, hsa-miR-16-5p, hsa-miR-17-3p, hsa-miR-1825, hsa-miR-186-5p, hsa-miR-18a-5p, hsa-miR-18b-3p, hsa-miR-191-5p, hsa-miR-195-5p, hsa-miR-199a-1-3p, hsa-miR-200b-3p, hsa-miR-203a-3p, hsa-miR-204-5p, hsa-miR-208a-3p, hsa-miR-21-5p, hsa-miR-210-3p, hsa-miR-211-5p, hsa-miR-215-5p, hsa-miR-216a-5p, hsa-miR-223-3p, hsa-miR-23a-3p, hsa-miR-24-3p, hsa-miR-299-5p, hsa-miR-30a-3p, hsa-miR-30c-5p, hsa-miR-30d-5p, hsa-miR-320a-3p, hsa-miR-323a-3p, hsa-miR-3615, hsa-miR-377-3p, hsa-miR-378a-3p, hsa-miR-378h, hsa-miR-382-5p, hsa-miR-411-5p, hsa-miR-423-5p, hsa-miR-4286, hsa-miR-449b-5p, hsa-miR-449c-5p, hsa-miR-451a, hsa-miR-484, hsa-miR-487a-5p, hsa-miR-494-3p, hsa-miR-499a-5p, hsa-miR-500a-3p, hsa-miR-625-5p, hsa-miR-877-5p, hsa-miR-92b-3p, hsa-miR-93-5p, hsa-mir19a-5p, and hsa-mir208a-3p. In some exemplary embodiments, the target RNA molecules comprise one or more miRNA molecules set forth in Tables 2, 3, and/or 4 herein.

In some exemplary embodiments, the target RNA molecules comprise one or more miRNA molecules selected from the group consisting of cel-miR-39-3p, hsa-Let-7a-5p, hsa-Let-7d-3p, hsa-Let-7i-5p, hsa-miR-1224-5p, hsa-miR-125b-5p, hsa-miR-126-3p, hsa-miR-1281, hsa-miR-130a-3p, hsa-mir-135a1-5p, hsa-miR-142-3p, hsa-miR-145-3p, hsa-miR-145-5p, hsa-miR-146a-5p, hsa-miR-155-5p, hsa-miR-15a-5p, hsa-miR-15b-5p, hsa-miR-16-5p, hsa-miR-17-3p, hsa-miR-1825, hsa-miR-186-5p, hsa-miR-18a-5p, hsa-miR-18b-3p, hsa-miR-191-5p, hsa-miR-195-5p, hsa-miR-199a-1-3p, hsa-miR-200b-3p, hsa-miR-203a-3p, hsa-miR-204-5p, hsa-miR-208a-3p, hsa-miR-21-5p, hsa-miR-210-3p, hsa-miR-211-5p, hsa-miR-215-5p, hsa-miR-216a-5p, hsa-miR-223-3p, hsa-miR-23a-3p, hsa-miR-24-3p, hsa-miR-299-5p, hsa-miR-30a-3p, hsa-miR-30c-5p, hsa-miR-30d-5p, hsa-miR-320a-3p, hsa-miR-323a-3p, hsa-miR-3615, hsa-miR-377-3p, hsa-miR-378a-3p, hsa-miR-378h, hsa-miR-382-5p, hsa-miR-411-5p, hsa-miR-423-5p, hsa-miR-4286, hsa-miR-449b-5p, hsa-miR-449c-5p, hsa-miR-451a, hsa-miR-484, hsa-miR-487a-5p, hsa-miR-494-3p, hsa-miR-499a-5p, hsa-miR-500a-3p, hsa-miR-625-5p, hsa-miR-877-5p, hsa-miR-92b-3p, hsa-miR-93-5p, hsa-mir19a-5p, hsa-mir208a-3p, hsa-miR-101-3p, hsa-miR-136-3p, hsa-miR-185-3p, hsa-miR-192-3p, hsa-miR-194-5p, hsa-miR-196b-5p, hsa-miR-214-3p, hsa-miR-339-3p, hsa-miR-4746-5p, hsa-miR-500a-5p, hsa-miR-539-5p, hsa-miR-576-5p, hsa-miR-1-3p, hsa-miR-1277-5p, hsa-miR-139-5p, hsa-miR-146b-5p, hsa-miR-183-5p, hsa-miR-188-5p, hsa-miR-190a-5p, hsa-miR-200a-3p, hsa-miR-205-5p, hsa-miR-2115-3p, hsa-miR-329-3p, hsa-miR-3690, hsa-miR-376a-3p, hsa-miR-376b-3p, hsa-miR-412-5p, hsa-miR-449a, hsa-miR-539-3p, hsa-miR-551a, hsa-miR-582-3p, hsa-miR-628-5p, hsa-miR-629-5p, hsa-miR-642a-5p, hsa-miR-651-5p, hsa-miR-873-5p, hsa-miR-887-3p, and hsa-miR-376c-3p.

In some exemplary embodiments, the target RNA molecules comprise one or more miRNA molecules selected from the group consisting of hsa-miR-101-3p, hsa-miR-136-3p, hsa-miR-17-3p, hsa-miR-185-3p, hsa-miR-192-3p, hsa-miR-194-5p, hsa-miR-196b-5p, hsa-miR-214-3p, hsa-miR-339-3p, hsa-miR-4746-5p, hsa-miR-500a-5p, hsa-miR-539-5p, hsa-miR-576-5p, hsa-miR-1-3p, hsa-miR-1277-5p, hsa-miR-139-5p, hsa-miR-146b-5p, hsa-miR-183-5p, hsa-miR-188-5p, hsa-miR-190a-5p, hsa-miR-195-5p, hsa-miR-200a-3p, hsa-miR-205-5p, hsa-miR-2115-3p, hsa-miR-215-5p, hsa-miR-223-3p, hsa-miR-29b-3p, hsa-miR-329-3p, hsa-miR-3690, hsa-miR-376a-3p, hsa-miR-376b-3p, hsa-miR-412-5p, hsa-miR-449a, hsa-miR-449c-5p, hsa-miR-539-3p, hsa-miR-551a, hsa-miR-582-3p, hsa-miR-628-5p, hsa-miR-629-5p, hsa-miR-642a-5p, hsa-miR-651-5p, hsa-miR-873-5p, hsa-miR-887-3p, and hsa-miR-376c-3p.

In some exemplary embodiments, the target RNA molecules comprise one or more miRNA molecules selected from the group consisting of hsa-miR-92b-5p, hsa-miR-6734-5p, hsa-miR-664a-5p, hsa-miR-576-5p, hsa-miR-539-5p, hsa-miR-500a-5p, hsa-miR-4488, hsa-miR-381-3p, hsa-miR-376c-3p, hsa-miR-339-3p, hsa-miR-185-3p, hsa-miR-17-3p, hsa-miR-1271-5p, and ‘hsa-miR-101.

In some exemplary embodiments, the target RNA molecules comprise one or more miRNA molecules selected from the group consisting of hsa-miR-99b-5p, hsa-miR-660-3p, hsa-miR-500a-5p, hsa-miR-4746-5p, hsa-miR-410-3p, hsa-miR-214-3p, hsa-miR-196b-5p, hsa-miR-194-5p, hsa-miR-192-3p, hsa-miR-185-3p, hsa-miR-17-3p, hsa-miR-136-3p, and hsa-miR-101-3p.

In some exemplary embodiments, the target RNA molecule is hsa-miR-17-3p.

In some exemplary embodiments, the target RNA molecules comprise one or more miRNA molecules selected from the group consisting of hsa-miR-5695, hsa-miR-454-5p, hsa-miR-3912-3p, hsa-miR-363-3p, hsa-miR-27a-5p, hsa-miR-191-5p, hsa-miR-17-3p, hsa-miR-145-5p, and hsa-let-7i-3p.

In some exemplary embodiments, the target RNA molecules comprise one or more miRNA molecules selected from the group consisting of hsa-miR-652-3p, hsa-miR-584-5p, hsa-miR-378a-3p, hsa-miR-338-3p, hsa-miR-320a-3p, hsa-miR-29c-3p, hsa-miR-221-3p, hsa-miR-20a-5p, hsa-miR-199b-3p, hsa-miR-181a-5p, hsa-miR-17-5p, hsa-miR-17-3p, hsa-miR-151a-5p, hsa-miR-151a-3p, hsa-miR-148a-3p, and hsa-miR-143-3p.

In some exemplary embodiments, the target RNA molecules comprise one or more miRNA molecules selected from the group consisting of ‘hsa-miR-576-5p, hsa-miR-539-5p, hsa-miR-500a-5p, hsa-miR-376c-3p, hsa-miR-339-3p, hsa-miR-185-3p, hsa-miR-17-3p, and hsa-miR-101-3p.

In some exemplary embodiments, the target RNA molecules comprise one or more miRNA molecules selected from the group consisting of hsa-miR-17-3p, hsa-miR-500a-5p, hsa-miR-215-5p, hsa-miR-1271-5p, and hsa-miR-151a-5p.

In some exemplary embodiments, the target RNA molecules comprise one or more miRNA molecules selected from the group consisting of hsa-miR-17-3p, hsa-miR-500a-5p, and hsa-miR-151a-5p.

In some exemplary embodiments, the target RNA molecules comprise one or more miRNA molecules selected from the group consisting of cel-miR-39-3p, hsa-Let-7a-5p, hsa-Let-7d-3p, hsa-Let-7i-5p, hsa-miR-1224-5p, hsa-miR-125b-5p, hsa-miR-126-3p, hsa-miR-1281, hsa-miR-130a-3p, hsa-mir-135a1-5p, hsa-miR-142-3p, hsa-miR-145-3p, hsa-miR-145-5p, hsa-miR-146a-5p, hsa-miR-155-5p, hsa-miR-15a-5p, hsa-miR-15b-5p, hsa-miR-16-5p, hsa-miR-17-3p, hsa-miR-1825, hsa-miR-186-5p, hsa-miR-18a-5p, hsa-miR-18b-3p, hsa-miR-191-5p, hsa-miR-195-5p, hsa-miR-199a-1-3p, hsa-miR-200b-3p, hsa-miR-203a-3p, hsa-miR-204-5p, hsa-miR-208a-3p, hsa-miR-21-5p, hsa-miR-210-3p, hsa-miR-211-5p, hsa-miR-215-5p, hsa-miR-216a-5p, hsa-miR-223-3p, hsa-miR-23a-3p, hsa-miR-24-3p, hsa-miR-299-5p, hsa-miR-30a-3p, hsa-miR-30c-5p, hsa-miR-30d-5p, hsa-miR-320a-3p, hsa-miR-323a-3p, hsa-miR-29b-3p, hsa-miR-3615, hsa-miR-377-3p, hsa-miR-378a-3p, hsa-miR-378h, hsa-miR-382-5p, hsa-miR-411-5p, hsa-miR-423-5p, hsa-miR-4286, hsa-miR-449b-5p, hsa-miR-449c-5p, hsa-miR-451a, hsa-miR-484, hsa-miR-487a-5p, hsa-miR-494-3p, hsa-miR-499a-5p, hsa-miR-500a-3p, hsa-miR-625-5p, hsa-miR-877-5p, hsa-miR-92b-3p, hsa-miR-93-5p, hsa-mir19a-5p, hsa-mir208a-3p, hsa-miR-101-3p, hsa-miR-136-3p, hsa-miR-192-3p, hsa-miR-194-5p, hsa-miR-196b-5p, hsa-miR-214-3p, hsa-miR-339-3p, hsa-miR-4746-5p, hsa-miR-500a-5p, hsa-miR-539-5p, hsa-miR-576-5p, hsa-miR-1-3p, hsa-miR-1277-5p, hsa-miR-139-5p, hsa-miR-146b-5p, hsa-miR-183-5p, hsa-miR-188-5p, hsa-miR-190a-5p, hsa-miR-200a-3p, hsa-miR-205-5p, hsa-miR-2115-3p, hsa-miR-3690, hsa-miR-376a-3p, hsa-miR-376b-3p, hsa-miR-412-5p, hsa-miR-449a, hsa-miR-539-3p, hsa-miR-551a, hsa-miR-582-3p, hsa-miR-628-5p, hsa-miR-629-5p, hsa-miR-642a-5p, hsa-miR-651-5p, hsa-miR-873-5p, hsa-miR-887-3p, hsa-miR-376c-3p, hsa-miR-92b-5p, hsa-miR-6734-5p, hsa-miR-664a-5p, hsa-miR-4488, hsa-miR-381-3p, hsa-miR-1271-5p, hsa-miR-101, hsa-miR-99b-5p, hsa-miR-660-3p, hsa-miR-329-3p, hsa-miR-410-3p, hsa-miR-185-3p, hsa-miR-569, hsa-miR-454-5p, hsa-miR-3912-3p, hsa-miR-363-3p, hsa-miR-27a-5p, hsa-miR-191-5p, hsa-miR-145-5p, and hsa-let-7i-3p, ‘hsa-miR-652-3p’, ‘hsa-miR-584-5p, hsa-miR-378a-3p, hsa-miR-338-3p, hsa-miR-29c-3p, hsa-miR-221-3p, hsa-miR-20a-5p, hsa-miR-199b-3p, hsa-miR-181a-5p, hsa-miR-17-5p, hsa-miR-151a-3p, hsa-miR-148a-3p, hsa-miR-143-3p, and hsa-miR-151a-5p.

In some exemplary embodiments, the methods disclosed herein further comprise preferentially enriching the RNA at a plurality of biomarkers indicative of transplant rejection. In some exemplary embodiments, the RNA biomarkers indicate an increased immune response, or a decreased immune response.

In some exemplary embodiments, the biomarkers indicative of a cancer, recurrence or metastasis of the cancer, an immune disease or disorder, pre-eclampsia, or congenital heart disease (CHD).

In some exemplary embodiments, the biomarkers comprise single nucleotide polymorphism (SNP) loci.

Samples and Methods for Isolating Nucleic Acids from the Samples

In some exemplary embodiments, the nucleic acid sample includes fragmented or digested nucleic acids. In some exemplary embodiments, the nucleic acid sample includes DNA, such as genomic DNA, cDNA, cell-free DNA (cfDNA), cell-free mitochondrial DNA (cf mDNA), cell-free DNA that originated from nuclear DNA (cf nDNA), cellular DNA, or mitochondrial DNA.

In some exemplary embodiments, nucleic acid sample includes RNA, such as cfRNA, cellular RNA, cytoplasmic RNA, coding cytoplasmic RNA, non-coding cytoplasmic RNA, mRNA, miRNA, mitochondrial RNA, rRNA, or tRNA. In some exemplary embodiments, the nucleic acid sample includes DNA from a single cell, 2 cells, 3 cells, 4 cells, 5 cells, 6 cells, 7 cells, 8 cells, 9 cell, 10 cells, or more than 10 cells. In some exemplary embodiments, the nucleic acid sample is a blood or plasma sample that is substantially free of cells. In some exemplary embodiments, the nucleic acid sample includes or is derived from blood, plasma, saliva, semen, sperm, cell culture supernatant, mucus secretion, dental plaque, gastrointestinal tract tissue, stool, urine, hair, bone, body fluids, tears, tissue, skin, fingernails, blastomeres, embryos, amniotic fluid, chorionic villus samples, bile, lymph, cervical mucus, or a forensic sample. In some exemplary embodiments, the target loci are segments of human nucleic acids. In some exemplary embodiments, the target loci are segments of human nucleic acids found in the human genome. In some exemplary embodiments, the target loci comprise or consist of single nucleotide polymorphisms (SNPs). In some exemplary embodiments, the primers are DNA molecules.

In some exemplary embodiments, the method includes isolating or purifying the DNA and/or RNA. There are a number of standard procedures known in the art to accomplish such an end. In some exemplary embodiments, the sample may be centrifuged to separate various layers. In some exemplary embodiments, the DNA or RNA may be isolated using filtration. In some exemplary embodiments, the preparation of the DNA or RNA may involve amplification, separation, purification by chromatography, liquid separation, isolation, preferential enrichment, preferential amplification, targeted amplification, or any of a number of other techniques either known in the art or described herein. In some exemplary embodiments for the isolation of DNA, RNase is used to degrade RNA. In some exemplary embodiments for the isolation of RNA, DNase (such as DNase I from Invitrogen, Carlsbad, Calif., USA) is used to degrade DNA. In some exemplary embodiments, an RNeasy™ mini kit (Qiagen), is used to isolate RNA according to the manufacturer's protocol. In some exemplary embodiments, small RNA molecules are isolated using the mirVana™ PARIS kit (Ambion, Austin, Tex., USA) according to the manufacturer's protocol (Gu et al., J. Neurochem. 122:641-649, 2012, which is hereby incorporated by reference in its entirety). The concentration and purity of RNA may optionally be determined using Nanovue (GE Healthcare, Piscataway, N.J., USA), and RNA integrity may optionally be measured by use of the 2100 Bioanalyzer (Agilent Technologies, Santa Clara, Calif., USA) (Gu et al., J. Neurochem. 122:641-649, 2012, which is hereby incorporated by reference in its entirety). In some exemplary embodiments, TRIZOL or RNAlater™ (Ambion) is used to stabilize RNA during storage.

In some exemplary embodiments, universal tagged adaptors are added to make a library. Prior to ligation, sample DNA may be blunt ended, and then a single adenosine base is added to the 3-prime end. In some exemplary embodiments, ligation of adaptors to nucleic acids is a sticky end ligation. Prior to ligation the DNA may be cleaved using a restriction enzyme or some other cleavage method. During ligation the 3-prime adenosine of the sample fragments and the complementary 3-prime tyrosine overhang of adaptor can enhance ligation efficiency. In some exemplary embodiments, adaptor ligation is performed using the ligation kit found in the AGILENT SURESELECT™ kit. In some exemplary embodiments, the library is amplified using universal primers. In an embodiment, the amplified library is fractionated by size separation or by using products such as AGENCOURT AMPURE™ beads or other similar methods. In some exemplary embodiments, PCR amplification is used to amplify target loci. In some exemplary embodiments, the amplified DNA is sequenced (such as sequencing using an ILLUMINA IIGAX or HiSeq sequencer). In some exemplary embodiments, the amplified DNA is sequenced from each end of the amplified DNA to reduce sequencing errors. If there is a sequence error in a particular base when sequencing from one end of the amplified DNA, there is less likely to be a sequence error in the complementary base when sequencing from the other side of the amplified DNA (compared to sequencing multiple times from the same end of the amplified DNA).

In some exemplary embodiments, miRNA can be separated from fragments of RNA caused by degradation because degraded RNA has lost phosphorylation groups at the ends. The miRNA retains the phosphorylation groups at the ends. An adapter can be ligated to phosphorylated miRNA ends, but the adaptor will not ligate to unphosphorylated RNA species such as degraded mRNA. The adaptor can contain sequences that allow for primer binding to aid reverse transcription to produce complementary DNA (cDNA) selectively from.

As nonlimiting examples, a locus can be a single nucleotide polymorphism, an intron, or an exon. In some exemplary embodiments, a locus can include an insertion, deletion, or transposition. In some exemplary embodiments, the sample can include a blood, sera, or plasma sample. In some exemplary embodiments, the sample can include free floating DNA (e.g. circulating cell-free tumor DNA or circulating cell-free fetal DNA) in a blood, sera, or plasma sample. In these embodiments, the sample is typically from an animal, such as a mammal or human, and is typically present in fragments about 160 nucleotides in length. In some exemplary embodiments, the free-floating DNA is isolated from blood using an EDTA-2Na tube after removal of cellular debris and platelets by centrifugation. The plasma samples can be stored at −80.degree. C. until the DNA is extracted using, for example, QIAamp™ DNA Mini Kit (Qiagen, Hilden, Germany), (e.g. Hamakawa et al., Br J Cancer. 2015; 112:352-356). However, the sample can be derived from other sources and nucleic acid molecules from any organism can be used for this method. In some exemplary embodiments, DNA derived from bacteria and/or viruses can be used to analyze true sequence variants within a mixed population, especially in environmental and biodiversity sampling.

Many kits and methods are known in the art for generating libraries of nucleic acid molecules for subsequent sequencing. Kits especially adapted for preparing libraries from small nucleic acid fragments, especially circulating cell-free DNA, can be useful for practicing methods provided herein. For example, the NEXTflex™ Cell Free kits (Bioo Scientific, Austin, Tex.) or the Natera Library Prep Kit (Natera, San Carlos, Calif.). Such kits would typically be modified to include adaptors that are customized for the amplification and sequencing steps of the methods provided herein. Adaptor ligation can also be performed using commercially available kits such as the ligation kit found in the Agilent SureSelect™ kit (Agilent, Santa Clara, Calif.).

Sample nucleic acid molecules are composed of naturally occurring or non-naturally occurring ribonucleotides or deoxyribonucleotides linked through phosphodiester linkages. Furthermore, sample nucleic acid molecules are composed of a nucleic acid segment that is targeted for sequencing. Sample nucleic acid molecules can be or can include nucleic acid segments that are at least 20, 25, 50, 75, 100, 125, 150, 200, 250, 300, 400, 500, 600, 700, 800, 900, or 1,000 nucleotides in length. In any of the embodiments disclosed herein the sample nucleic acid molecules or nucleic acid segments can be between 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, and 500 nucleotides in length on the low end of the range and 10, 11, 12, 13, 14, 15, 16, 17, 18, 19, 20, 25, 50, 75, 100, 125, 150, 200, 250, 300, 400, 500, 1,000, 2,000, 3,000, 4,000, 5,000, 6,000, 7,000, 8,000, 9,000, and 10,000 nucleotides in length on the high end of the range. In some exemplary embodiments, the nucleic acid molecules can be fragments of genomic DNA and can be between 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, and 500 nucleotides in length on the low end of the range and 10, 11, 12, 13, 14, 15, 16, 17, 18, 19, 20, 25, 50, 75, 100, 125, 150, 200, 250, 300, 400, 500, 1,000, 2,000, 3,000, 4,000, 5,000, 6,000, 7,000, 8,000, 9,000, and 10,000 nucleotides in length on the high end of the range. For the sake of clarity, nucleic acids initially isolated from a living tissue, fluid, or cultured cells, can be much longer than sample nucleic acid molecules processed using methods herein. As discussed herein, for example, such initially isolated nucleic acid molecules can be fragmented to generate nucleic acid segments, before being used in the methods herein. In some exemplary embodiments, the nucleic acid molecules and nucleic acid segments can be identical. The sample nucleic acid molecule or sample nucleic acid segment can include a target locus that contains the nucleotide or nucleotides that are being queried, especially a single nucleotide polymorphism or single nucleotide variant. In any of the disclosed embodiments, the target loci can be at least 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, 500, 600, 700, 800, 900, or 1,000 nucleotides in length and include a portion of or the entirety of the sample nucleic acid molecule and/or the sample nucleic acid segment. In other embodiments, the target loci can be between 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, and 500 nucleotides in length on the low end of the range and 10, 11, 12, 13, 14, 15, 16, 17, 18, 19, 20, 25, 50, 75, 100, 125, 150, 200, 250, 300, 400, 500, 1,000, 2,000, 3,000, 4,000, 5,000, 6,000, 7,000, 8,000, 9,000, and 10,000 nucleotides in length on the high end of the range. In some exemplary embodiments, the target loci on different sample nucleic acid molecules can be at least 50%, 60%, 70%, 80%, 90% 95%, 96%, 97%, 98%, 99%, 99.9%, or 100% identical. In some exemplary embodiments, the target loci on different sample nucleic acid molecules can share at least 50%, 60%, 70%, 80%, 90% 95%, 96%, 97%, 98%, 99%, 99.9%, or 100% sequence identity.

In some exemplary embodiments, the entire sample nucleic acid molecule is a sample nucleic acid segment. For example, in certain embodiments where adaptors are ligated directly to the ends of sample nucleic acid molecules, or ligated to a nucleic acid(s) ligated to the ends of sample nucleic acid molecules, or ligated as part of primers that bind to sequences at the termini of sample nucleic acid segments, or adapters, such as universal adapters added thereto, as discussed further herein, the entire nucleic acid molecule can be a sample nucleic acid segment. In other embodiments, for example certain embodiments where adaptors are attached to sample nucleic acid molecules as part of primers that target binding sites internal to the termini of sample nucleic acid molecules, a portion of the sample nucleic acid molecule can be the sample nucleic acid segment that is targeted for downstream sequencing. For example, at least 50%, 60%, 70%, 75%, 80%, 85%, 90%, 95%, 96%, 97%, 98%, 99%, or 100% of a sample nucleic acid molecule can be a nucleic acid segment.

In some exemplary embodiments, sample nucleic acid molecules are a mixture of nucleic acids isolated from a natural source, some sample nucleic acid molecules having identical sequences, some having sequences sharing at least 50%, 60%, 70%, 80%, 90%, 95%, 98%, or 99% sequence identity, and some with less than 50%, 40%, 30%, 20%, 10%, or 5% sequence identity over between 20, 25, 50, 75, 100, 125, 150, 200, 250 nucleotides on the low end of the range, and 50, 75, 100, 125, 150, 200, 250, 300, 400, or 500 nucleotides on the high end of the range. Such sample nucleic acid molecules can be nucleic acid samples isolated from tissues or fluids of a mammal, such as a human, without enriching one sequence over another. In other embodiments, target sequences, for example, those from a gene of interest, can be enriched prior to performing methods provided herein.

### Removing Contamination

Biological samples may contain large amounts of contaminating nucleic acids that can make detection of target nucleic acids difficult, reduce quality of the nucleic acid library, and/or reduce the throughput of the assay. For example, blood cells within a blood sample may burst and shed nucleic acids into the sample, resulting in that the sample may comprise large amount of irrelevant nucleic acids form the red blood cells. In one aspect of the present disclosure, the genome editing system CRISPR (clustered regularly interspersed short palindromic repeats) may be used to remove contaminating nucleic acids. CRISPR-Cas technology allows for simple, flexible, and relatively inexpensive cutting of DNA in a targeted sequence-specific manner. The required components for in vitro digestion are a Cas and a target-specific guide RNA (gRNA).), therefore relatively high concentrations of enzyme must be used for complete digestion.

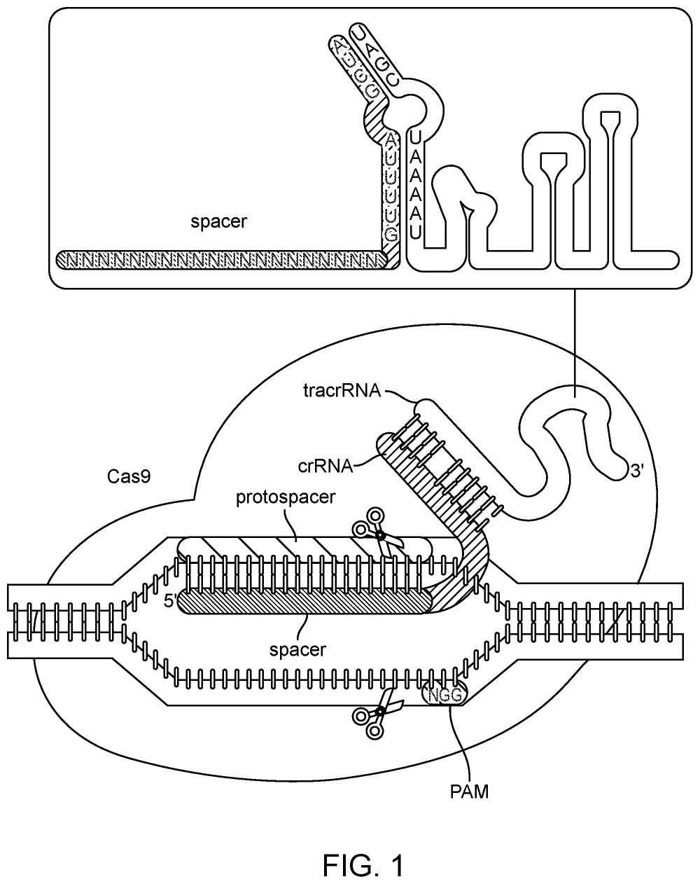

An effective CRISPR-Cas complex must have several features. First, the gRNA must have a spacer element which is complementary to the target nucleic acids. This complementary target nucleic acid sequence is referred to as the protospacer. In addition, immediately downstream of the spacer (For Cas9, for Cas12a it is immediately upstream of the spacer), there must be a Protospacer Adjacent Motif (PAM). For Cas9 this motif is NGG, where N is any nucleotide, while for Cas12a the PAM motif is TTTV, where V is any nucleotide except T. Also required for proper complex formation is the core of the gRNA. However, since this is conserved for all gRNAs for the same Cas enzyme, IDT has standardized this for Cas9 and created a separate “tracrRNA” which anneals to the shorter “crRNA” to form the complete gRNA. The mechanism for CRISPR process is graphically depicted in FIG. 1.

Various CRISPR Cas systems have been developed. The CRISPR Cas9 system is the first and best-characterized single-protein CRISPR effector and are subcategorized in to types II-A, II-B, and II-C. Cas9 makes a blunt double-stranded DNA break, which can then be repaired by either non-homologous end joining or homologous recombination with a donor template DNA to create site-specific edits. Type II-A Cas9s generally have high genome editing efficiency, but off-target cleavage at unintended genome sites can be a disadvantage. Variants have been engineered to overcome these limitations, and type II-C Cas9s tend to have naturally higher fidelity. Cas9 uses a guide spacer with a length of 18-24 nucleotides (nt). The total length of the guide for Cas9 may be about 100 nt. The PAM sequence may be 3-NGG (SpCas9), 3-NNGRRT (SaCas9), 3-NNNNGATT (NmCas9). Cas9 produces a Blunt-ended dsDNA break. Mutant Cas9 enzymes are also commercially available which produce single-stranded nicks in DNA on one strand of the target, as well as a non-cutting mutant which only binds the target.

Cas12 belongs to Type V CRISPR-Cas, which comprises subtypes V-A and V-B, and is also known as Cpf1 (Type V-A), or C2cl (type V-B). Cas12 is a compact and efficient enzyme that creates staggered cuts in dsDNA, and thereby creates a 3 to 5 base overhang. Guide spacer of Cas 12 has a length of 18-25 nt, and the total guide length is 42-44 nt. The Cas 12 PAM sequences May 5-TTTN (FnCas12a). Cas12 processes its own guide RNAs, leading to increased multiplexing ability. Cas 12 has also been engineered as a platform for epigenome editing, and it was recently discovered that Cas12a can indiscriminately chop up single-stranded DNA once activated by a target DNA molecule matching its spacer sequence.

Cas 13 is a Type VI CRISPR-Cas that comprises subtypes VI-A, VI-B, VI-C, and VI-D, and is also known as C2c2 or CasRx (type VI-D). Cas 13 targets RNA, not DNA. The guide spacer for Cas13 has a length of 22-30 nt, and a total guide length of 52-66 nt. The Cas13 PAM sequences included3-H (LshCas 13a), 5-D and 3-NAN or NNA (BzCas 13b), and none (RfCas13d). Once Cas13 is activated by a ssRNA sequence bearing complementarity to its crRNA spacer, Cas13 exhibits a nonspecific RNase activity to destroy all nearby RNA regardless of their sequence. This property has been harnessed in vitro for precision diagnostics. These systems can also be used for efficient, multiplexable, and specific RNA knockdown or RNA sequence editing in mammalian cells.

The methods disclosed herein may comprise utilizing any CRISPR-Cas System to deplete contaminating or overabundant nucleic acids in a biological sample or libraries. For example Cas9 and/or Cas12 may be used to remove DNA species in whole blood or hemolysis tainted blood, serum or plasma samples, thereby increasing the sensitivity of detecting the target loci. In another aspect, the Cas enzymes targeting RNA species directly may be used. For example, Cas13 may be used to remove contaminating RNA species from the biological sample prior to library preparation.

Cas enzymes can be given a mixture of different gRNAs to allow targeting of many different loci in the same reaction. In some exemplary embodiments, 1 gRNA is used to target a loci. In some exemplary embodiments, 1-5 gRNAs are used to target 1-5 loci in the same reaction. In some exemplary embodiments, 1-10 gRNAs are used to target 1-10 loci in the same reaction. In some exemplary embodiments, 1-100 gRNAs are used to target 1-100 loci in the same reaction. In some exemplary embodiments, 1-5000 gRNAs are used to target 1-5000 loci in the same reaction. In some exemplary embodiments, 10-5000 gRNAs are used to target 10-5000 loci in the same reaction. In some exemplary embodiments, 100-5000 gRNAs are used to target 100-5000 loci in the same reaction. In some exemplary embodiments, 1000-50000 gRNAs are used to target 1000-50000 loci in the same reaction. In some exemplary embodiments, 10000-5000 gRNAs are used to target 10000-50000 loci in the same reaction. In some exemplary embodiments, a plurality of gRNAs are used to target a plurality of target loci in the same reaction. In some exemplary embodiments, at least 10 gRNAs are used to target at least 10 loci in the same reaction. In some exemplary embodiments, at least 20 gRNAs are used to target at least 20 loci in the same reaction. In some exemplary embodiments, at least 50 gRNAs are used to target at least 50 loci in the same reaction. In some exemplary embodiments, at least 100 gRNAs are used to target at least 100 loci in the same reaction. In some exemplary embodiments, at least 500 gRNAs are used to target at least 500 loci in the same reaction. In some exemplary embodiments, at least 1000 gRNAs are used to target at least 1000 loci in the same reaction. In some exemplary embodiments, at least 2000 gRNAs are used to target at least 2000 loci in the same reaction. In some exemplary embodiments, at least 3000 gRNAs are used to target at least 3000 loci in the same reaction. In some exemplary embodiments, at least 4000 gRNAs are used to target at least 4000 loci in the same reaction. In some exemplary embodiments, at least 5000 gRNAs are used to target at least 5000 loci in the same reaction. In some exemplary embodiments, at least 10000 gRNAs are used to target at least 10000 loci in the same reaction. In some exemplary embodiments, at least 20000 gRNAs are used to target at least 20000 loci in the same reaction.

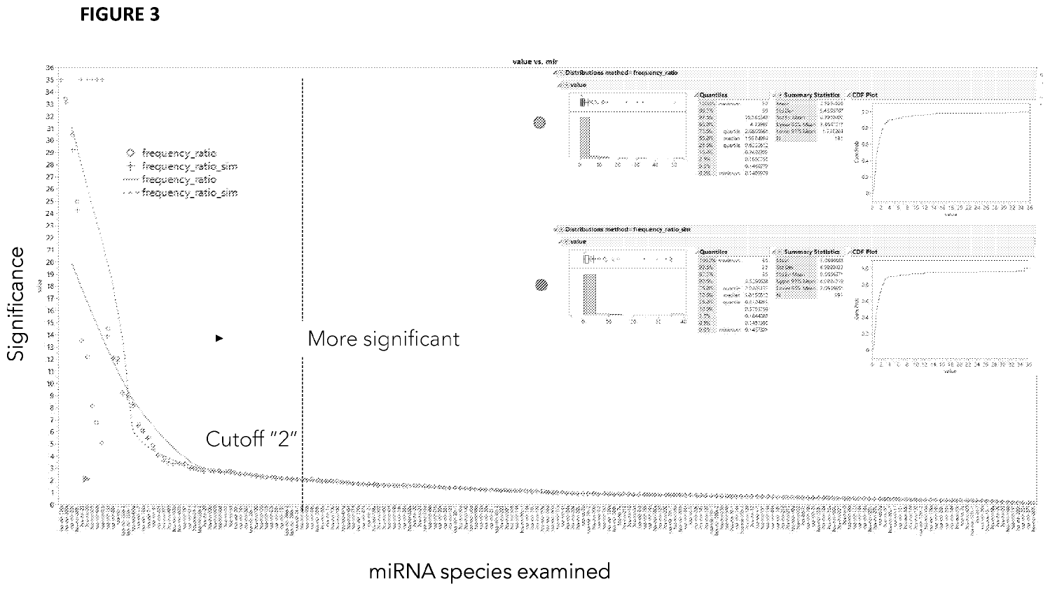

Untargeted miRNA analysis may be performed by ligating the miRNA to an adaptor as outlined. The adaptor may provide a NGG location adjacent to the miRNA to allow for Cas9 cutting. When an unwanted sequence is ligated to the adaptor, a Cas9-gRNA complex complementary to this unwanted sequence will be introduced (see FIG. 3). This will result in removal of the adaptor, and preventing PCR amplification. This step is performed on double-stranded reverse transcribed DNA.

In some exemplary embodiments, a pilot sequencing run can be used to inform which targets need to be removed from future sequencing runs. This would allow assay developers to focus on only miRNAs of interest by designing gRNAs targeting the most common uninteresting or contaminating small RNA fragments actually observed in the specific sample type used in the assay. This could include highly abundant miRNAs or small fragments of ribosomal or messenger RNAs that might cause background noise.

In some exemplary embodiments, Cas enzymes can be removed by using thermolabile Proteinase K (NEB P8111S) because Cas complexes are relatively long-lived and may interfere with downstream applications.

While this approach could also be used in targeted miRNA applications, it is preferable to improve/remove bad primers prior to the assay rather than try to remove them after amplification using CRISPR-Cas.

In some exemplary embodiments, the CRISPER-Cas mediated removal of contaminating species may also be used in mRNA applications. In some exemplary embodiments, CRISPR-Cas approaches are used to remove contaminating mRNA species with a length less than 20 nucleotides. When the length of the contaminating mRNA species is less than 20 nucleotides, a similar approach to that proposed for miRNA could be used to design assays to specifically target the observed sequences. This could be especially useful if, for example, barcoding primer dimers are consuming a large portion of the sequencing reads.

In some exemplary embodiments, CRISPR-Cas approaches are used to remove contaminating mRNA species with a length larger than 20 nucleotides. In some exemplary embodiments, CRISPR-Cas approaches are used in combination with a “tag and capture” method to remove contaminating mRNA species with a length larger than 20 nucleotides.

In some exemplary embodiments, the CRISPR-Cas method to remove contaminating species is used on cDNA derived from miRNA or mRNA prior to amplification. In some exemplary embodiments, the CRISPR-Cas method to remove contaminating species is used on cDNA derived from miRNA or mRNA, and wherein the cDNA is amplified for 1 to 5 cycles, 1 to 10 cycles, or 1-15, cycles. In some exemplary embodiments, the cDNA is amplified for no more than 2, 3, 4, 5, 6, 7, 8, 9, or 10 cycles prior to CRISPR-Cas mediated removal of contaminating species.

In some exemplary embodiments, the contaminating or overabundant species comprise hemoglobin mRNA, tRNA, rRNA, and miRNAs such as miR-451, miR-144, and miR-486, thereby increasing the fraction of desired reads that map to a target loci of interest per sample and the sample throughput per sequencing run.

In some exemplary embodiments, the method further comprises depleting adaptor dimers, primer dimers, unwanted ligation products from the composition of amplified nucleic acids comprising target loci, thereby increasing the fraction of desired reads that map to a target loci of interest per sample and the sample throughput per sequencing run.

Methods of identification of miRNA that bind to messenger RNAs of Organ Health transplant interest

Text mine a microRNA reference database to identify miRNA that bind to messenger RNAs of known interest to transplant health.

Predict the specificity of microRNA that bind messenger RNAs of known interest to transplant health.

Identify microRNAs by combining text mining of a miRNA reference database (a) and miRNA that bind mRNA of known interest (b). In the working examples herein, 7 higher specificity miRNAs were identified from combining method a and b.

Identify miRNA among large mRNA gene sets related to transplant organ health (such as the Banff 2019 expert guided gene set) and look for a common miRNA signature in that gene set versus randomly selected gene sets. The sets of mRNA may be at least 1000 mRNAs.

Artificial intelligence may be used for text mining and predicting miRNAs that bind mRNA of known interest.

Combining Measuring Cell-Free DNA with Measuring RNA to Determine And/or Monitoring Transplant Rejection

In some exemplary embodiments, the risk of cancer, cancer recurrence, or cancer metastasis may be determined based on: (i) measuring the amount of donor-derived cell-free DNA in a sample obtained from the transplant recipient, extracting cell-free DNA from the sample obtained from the transplant recipient, wherein the extracted cell-free DNA comprises donor-derived cell-free DNA and recipient-derived cell-free DNA; (ii) performing targeted amplification of the extracted DNA at 50-50,000 target loci in a single reaction volume; (iii) sequencing the amplified DNA by high-throughput sequencing to obtain sequencing reads and quantifying the amount of donor-derived cell-free DNA based on the sequencing reads, determining transplant rejection based on whether the amount of donor-derived cell-free DNA or a function thereof exceeds a cutoff threshold of cell-free DNA amount that indicates transplant rejection, wherein transplant rejection is determined based on whether both the amount of donor-derived RNA and the amount of donor derived cell-free DNA or function thereof exceeds a cutoff threshold that indicates transplant rejection. The combination of the amount of RNA and cfDNA in the samples obtained from the transplant recipient can serve as a biomarker for rejection and as a biomarker for net state of immunosuppression. In another aspect, a rejection risk for the transplant recipient can be determined based on the amount of donor-derived RNA and/or amount of cell-free DNA. In another aspect, a rejection risk for the transplant recipient can be determined based on the amount of donor-derived mRNA and/or amount of cell-free DNA. In another aspect, a rejection risk for the transplant recipient can be determined based on the amount of donor-derived miRNA and/or amount of cell-free DNA.

In some exemplary embodiments, the risk of cancer, cancer recurrence, or cancer metastasis may be determined based on: (i) measuring the amount of donor-derived cell-free DNA in a sample obtained from the subject suffering from or suspected to suffer from a cancer, extracting cell-free DNA from the sample obtained from the subject, wherein the extracted cell-free DNA comprises cell-free DNA derived from the tumor or cancer cells and cell-free DNA from normal tissue or cells; (ii) performing targeted amplification of the extracted DNA at 50-50,000 target loci in a single reaction volume; (iii) sequencing the amplified DNA by high-throughput sequencing to obtain sequencing reads and quantifying the amount of cell-free DNA derived from the tumor or cancer cells based on the sequencing reads, determining risk of cancer, cancer recurrence or cancer metastasis based on whether the amount of cell-free DNA derived from the tumor or cancer cells or a function thereof exceeds a cutoff threshold of cell-free DNA derived from the tumor or cancer cells amount that indicates cancer, cancer recurrence or cancer metastasis. In some embodiment, the combination of an amount of mRNA targets in combination with an amount of cfDNA in the samples indicates cancer, cancer recurrence or cancer metastasis.

In another aspect, a risk of cancer, cancer recurrence or cancer metastasis can be determined based on the amount of RNA and/or amount of cell-free DNA derived from a tumor or cancer cells.

### Determination of Rejection Risk for the Transplant Recipient

In some exemplary embodiments, wherein the rejection risk for the transplant recipient is determined using machine learning analysis such as logistic LASSO regression, Mann-Whitney test, Benjamini-Hochberg (BH) Procedure, Recursive Feature Elimination (RFE)—support vector machine (SVM), RFE-Random Forest, or gradient boosting. In some exemplary embodiments, the machine learning analysis incorporates the amount of donor-derived RNA in the sample of the transplant recipient or a function thereof as a parameter. In some exemplary embodiments, the machine learning analysis incorporates the number of reads of donor-derived RNA or a function thereof as a parameter. In some exemplary embodiments, the machine learning analysis incorporates the estimated percentage of donor-derived RNA out of total RNA as a parameter. In some exemplary embodiments, the machine learning analysis incorporates the amount of cell-free DNA, the number of reads of cell-free DNA, or the estimated percentage of cell-free DNA out of total cell-free DNA in the sample of the transplant recipient as a parameter. In some exemplary embodiments, the machine learning analysis further incorporates the amount of total cell-free DNA in the sample of the transplant recipient or a function thereof as a parameter. In some exemplary embodiments, the machine learning analysis further incorporates the number of reads of total cell-free DNA or a function thereof as a parameter.

Machine learning may be used to resolve rejection vs non-rejection. Machine learning is disclosed in WO2020/018522, titled “Methods and Systems for calling Ploidy States using a Neural Network” and filed on Jul. 16, 2019 as PCT/US2019/041981, which is incorporated herein by reference in its entirety. In some exemplary embodiments, the cutoff threshold value is scaled according to the amount of total cfDNA or RNA in the blood sample.

In some exemplary embodiments, the cutoff threshold value is expressed as percentage of dd-cfDNA (dd-cfDNA %) in the blood sample. In some exemplary embodiments, the cutoff threshold value is expressed as quantity or absolute quantity of dd-cfDNA. In some exemplary embodiments, the cutoff threshold value is expressed as quantity or absolute quantity of dd-cfDNA per volume unit of the blood sample. In some exemplary embodiments, the cutoff threshold value is expressed as quantity or absolute quantity of dd-cfDNA per volume unit of the blood sample multiplied by body mass, BMI, or blood volume of the transplant recipient.

In some exemplary embodiments, the cutoff threshold value takes into account the body mass, BMI, or blood volume of the patient. In some exemplary embodiments, the cutoff threshold value takes into account one or more of the following: donor genome copies per volume of plasma, cell-free DNA yield per volume of plasma, donor height, donor weight, donor age, donor gender, donor ethnicity, donor organ mass, donor organ, live vs deceased donor, the donor's familial relationship to the recipient (or lack thereof), recipient height, recipient weight, recipient age, recipient gender, recipient ethnicity, creatinine, eGFR (estimated glomerular filtration rate), cfDNA methylation, DSA (donor-specific antibodies), KDPI (kidney donor profile index), medications (immunosuppression, steroids, blood thinners, etc.), infections (BKV, EBV, CMV, UTI), recipient and/or donor HLA alleles or epitope mismatches, Banff classification of renal allograft pathology, and for-cause vs surveillance or protocol biopsy.

In some exemplary embodiments, the method has a sensitivity of at least 50% in identifying acute rejection (AR) over non-AR when the dd-cfDNA amount is above the cutoff threshold value scaled or adjusted according to the amount of total cfDNA in the blood sample and a confidence interval of 95%. In some exemplary embodiments, the method has a sensitivity of at least 60% in identifying acute rejection (AR) over non-AR when the dd-cfDNA amount is above the cutoff threshold value scaled or adjusted according to the amount of total cfDNA in the blood sample and a confidence interval of 95%. In some exemplary embodiments, the method has a sensitivity of at least 70% in identifying acute rejection (AR) over non-AR when the dd-cfDNA amount is above the cutoff threshold value scaled or adjusted according to the amount of total cfDNA in the blood sample and a confidence interval of 95%. In some exemplary embodiments, the method has a sensitivity of at least 80% in identifying acute rejection (AR) over non-AR when the dd-cfDNA amount is above the cutoff threshold value scaled or adjusted according to the amount of total cfDNA in the blood sample and a confidence interval of 95%. In some exemplary embodiments, the method has a sensitivity of at least 85% in identifying acute rejection (AR) over non-AR when the dd-cfDNA amount is above the cutoff threshold value scaled or adjusted according to the amount of total cfDNA in the blood sample and a confidence interval of 95%. In some exemplary embodiments, the method has a sensitivity of at least 90% in identifying acute rejection (AR) over non-AR when the dd-cfDNA amount is above the cutoff threshold value scaled or adjusted according to the amount of total cfDNA in the blood sample and a confidence interval of 95%. In some exemplary embodiments, the method has a sensitivity of at least 95% in identifying acute rejection (AR) over non-AR when the dd-cfDNA amount is be above the cutoff threshold value scaled or adjusted according to the amount of total cfDNA in the blood sample and a confidence interval of 95%.

In some exemplary embodiments, the method has a specificity of at least 50% in identifying acute rejection (AR) over non-AR when the dd-cfDNA amount is above the cutoff threshold value scaled or adjusted according to the amount of total cfDNA in the blood sample and a confidence interval of 95%. In some exemplary embodiments, the method has a specificity of at least 60% in identifying acute rejection (AR) over non-AR when the dd-cfDNA amount is above the cutoff threshold value scaled or adjusted according to the amount of total cfDNA in the blood sample and a confidence interval of 95%. In some exemplary embodiments, the method has a specificity of at least 70% in identifying acute rejection (AR) over non-AR when the dd-cfDNA amount is above the cutoff threshold value scaled or adjusted according to the amount of total cfDNA in the blood sample and a confidence interval of 95%. In some exemplary embodiments, the method has a specificity of at least 75% in identifying acute rejection (AR) over non-AR when the dd-cfDNA amount is above the cutoff threshold value scaled or adjusted according to the amount of total cfDNA in the blood sample and a confidence interval of 95%. In some exemplary embodiments, the method has a specificity of at least 80% in identifying acute rejection (AR) over non-AR when the dd-cfDNA amount is above the cutoff threshold value scaled or adjusted according to the amount of total cfDNA in the blood sample and a confidence interval of 95%. In some exemplary embodiments, the method has a specificity of at least 85% in identifying acute rejection (AR) over non-AR when the dd-cfDNA amount is above the cutoff threshold value scaled or adjusted according to the amount of total cfDNA in the blood sample and a confidence interval of 95%. In some exemplary embodiments, the method has a specificity of at least 90% in identifying acute rejection (AR) over non-AR when the dd-cfDNA amount is above the cutoff threshold value scaled or adjusted according to the amount of total cfDNA in the blood sample and a confidence interval of 95%. In some exemplary embodiments, the method has a specificity of at least 95% in identifying acute rejection (AR) over non-AR when the dd-cfDNA amount is above the cutoff threshold value scaled or adjusted according to the amount of total cfDNA in the blood sample and a confidence interval of 95%.

Some embodiments of the present invention relate to a method of quantifying the amount of donor-derived cell-free DNA in a biological sample of a transplant recipient, comprising: a) isolating cell-free DNA from the biological sample of the transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of donor-derived cell-free DNA and the amount of total cell-free DNA, wherein the amount of total cell-free DNA is quantified using sequencing reads derived from the first Tracer DNA composition.

Some embodiments use either a fixed threshold of donor DNA per plasma volume or one that is not fixed, such as adjusted or scaled as noted herein. The way that this is determined can be based on using a training data set to build an algorithm to maximize performance. It may also take into account other data such as patient weight, age, or other clinical factors.

In some exemplary embodiments, the method further comprises determining the occurrence or likely occurrence of transplant rejection using the amount of donor-derived cell-free DNA. In some exemplary embodiments, the amount of donor-derived cell-free DNA is compared to a cutoff threshold value to determine the occurrence or likely occurrence of transplant rejection, wherein the cutoff threshold value is adjusted or scaled according to the amount of total cell-free DNA. In some exemplary embodiments, the cutoff threshold value is a function of the number of reads of the donor-derived cell-free DNA.

In some exemplary embodiments, the method comprises applying a scaled or dynamic threshold metric that takes into account the amount of total cfDNA in the samples to more accurately assess transplant rejection. In some exemplary embodiments, the method further comprises flagging the sample if the amount of total cell-free DNA is above a pre-determined value. In some exemplary embodiments, the method further comprises flagging the sample if the amount of total cell-free DNA is below a pre-determined value.

The RNA or DNA may be extracted from a sample from the transplant recipient, wherein the sample comprises blood, plasma, serum, Cerebrospinal fluid (CSF), or urine.

In some exemplary embodiments, the machine learning analysis further incorporates time post-transplantation as a parameter. In some exemplary embodiments, the machine learning analysis further incorporates the age of transplant recipient and/or transplant donor as a parameter. In some exemplary embodiments, the machine learning analysis further incorporates the gender of transplant recipient and/or transplant donor as a parameter.

In some exemplary embodiments, the rejection risk for the transplant recipient is determined with a sensitivity of at least 0.81, or at least 0.82, or at least 0.83, or at least 0.84, or at least 0.85, or at least 0.86, or at least 0.87, or at least 0.88, or at least 0.89, or at least 0.90. In some exemplary embodiments, the rejection risk for the transplant recipient is determined with a specificity of at least 0.81, or at least 0.82, or at least 0.83, or at least 0.84, or at least 0.85, or at least 0.86, or at least 0.87, or at least 0.88, or at least 0.89, or at least 0.90. In some exemplary embodiments, the rejection risk for the transplant recipient is determined with an area under the curve (AUC) of at least at least 0.86, or at least 0.87, or at least 0.88, or at least 0.89, or at least 0.90, or at least 0.91 or at least 0.92, or at least 0.93, or at least 0.94, or at least 0.95.

### Methods for Measuring the Amount of Nucleic Acids

In some exemplary embodiments, the amount of RNA is measured by quantitative PCR. In some exemplary embodiments, the amount of RNA is measured by real-time PCR. In some exemplary embodiments, the amount of RNA is measured by digital PCR. In some exemplary embodiments, the amount of RNA is measured by sequencing such as high-throughput sequencing, next-generation sequence, or sequencing-by-synthesis.

In some exemplary embodiments, the amount of donor-derived nucleic acids (e.g. RNA and/or DNA) is determined by using ratiometric and/or machine learning-artificial intelligence comparisons at a single or a plurality of time points. In some exemplary embodiments, the amount of donor-derived mRNA is determined by using ratiometric and/or machine learning-artificial intelligence comparisons at a single or a plurality of time points. In some exemplary embodiments, the amount of donor-derived miRNA is determined by using ratiometric and/or machine learning-artificial intelligence comparisons at a single or a plurality of time points.

In some exemplary embodiments, the amount of RNA or cell-free DNA is measured by a quantitative PCR method. In some exemplary embodiments, the amount of mRNA is measured by a quantitative PCR method. In some exemplary embodiments, the amount of miRNA is measured by a quantitative PCR method. In some exemplary embodiments, the quantitative PCR method comprises real-time PCR or digital PCR.

In some exemplary embodiments, the amount of mRNA or cell-free DNA is measured by massively multiplex PCR (mmPCR) to obtain amplicons comprising biomarkers, and sequencing of the amplicons.

In some exemplary embodiments, the amount of nucleic acids (e.g. mRNA, miRNA, or cell-free DNA) is measured by using microarray.

In some exemplary embodiments, the amount of nucleic acids (e.g. mRNA, miRNA, or cell-free DNA) is measured by using molecular barcodes and microscopic imaging (such as NanoString nCounter®).

In some exemplary embodiments, the amount of donor-derived RNA is measured by: extracting RNA from the blood, plasma, serum, cerebral spinal fluid (CSF), or urine sample of the transplant recipient, wherein the extracted RNA comprises donor-derived RNA and recipient-derived RNA; performing targeted amplification of the extracted RNA at 100-50,000 target loci in a single reaction volume; sequencing the amplified RNA by high-throughput sequencing to obtain sequencing reads and quantifying the amount of donor-derived RNA based on the sequencing reads. In some exemplary embodiments, the amplification of RNA comprises performing reverse transcriptase to obtain complementary DNA (cDNA).

In some exemplary embodiments, the amount of donor-derived cell-free DNA is measured by: extracting cell-free DNA from the blood, plasma, serum cerebral spinal fluid (CSF), or urine sample of the transplant recipient, wherein the extracted cell-free DNA comprises donor-derived cell-free DNA and recipient-derived cell-free DNA; performing targeted amplification of the extracted DNA at 10-50,000 target loci in a single reaction volume; sequencing the amplified DNA by high-throughput sequencing to obtain sequencing reads and quantifying the amount of donor-derived cell-free DNA based on the sequencing reads.

In some exemplary embodiments, the method is performed without prior knowledge of donor genotypes. In some exemplary embodiments, the method does not comprise genotyping transplant donor(s).

In some exemplary embodiments, the amount of nucleic acids is measured by targeted amplification. In some exemplary embodiments, the amount of a particular mRNA target is measured by targeted amplification. In some exemplary embodiments, the targeted amplification comprises PCR. In some exemplary embodiments, the primers for the targeted amplification include 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 pairs of forward and reverse PCR primers. In some exemplary embodiments, the targeted amplification comprises performing amplification at 100-20,000, 500-20,000, 1,000-10,000, 200-500, 500-1,000, 1,000-2,000, 2,000-5,000, 5,000-10,000, 10,000-20,000, 20,000-50,000 target loci in a single reaction volume using 500-20,000, 1,000-10,000, 200-500, 500-1,000, 1,000-2,000, 2,000-5,000, 5,000-10,000, 10,000-20,000, or 20,000-50,000 primer pairs to obtain amplification products.

In some exemplary embodiments, the targeted amplification comprises nested PCR. In some exemplary embodiments, the primers for the targeted amplification include a first universal primer and 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 target-specific primers, and a second universal primer and 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 inner target-specific primers. In some exemplary embodiments, the targeted amplification comprises performing amplification at 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 target loci in a single reaction volume using a first universal primer and 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 target-specific primers to obtain amplification products. In some exemplary embodiments, the targeted amplification comprises performing amplification at 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 target loci in a single reaction volume using a second universal primer and 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 inner target-specific primers to obtain amplification products. In some exemplary embodiments, the methods disclosed herein comprise PCR amplification of at least 10, at least 100, at least 500, at least 1000, at least 2000 biomarkers, from 10-1000, 100-10000, 200-50000, or 500-20000 RNA biomarkers, using at least 10, at least 100, at least 500, at least 1000, at least 2000, from 10-1000, 100-10000, 200-50000, 500-20000 pairs of forward and reverse PCR primers. In some exemplary embodiments, step (b) comprises amplification of at least 2, at least 5, at least 10, at least 20, at least 30, at least 50, at least 100 target RNA molecules, from 2-10, 200-100, 50-500, or 50-2000 target RNA molecules, using at least at least 2, at least 5, at least 10, at least 20, at least 30, at least 50, at least 100 target RNA molecules, from 2-10, 200-100, 50-500, or 50-2000 pairs of forward and reverse PCR primers.

In some exemplary embodiments, the method further comprises attaching tags to the amplification products prior to performing high-throughput sequencing, wherein the tags comprise sequencing-compatible adaptors. In some exemplary embodiments, the method further comprises attaching tags to the extracted RNA prior to performing targeted amplification, wherein the tags comprise adaptors for amplification. In some embodiment, the tags comprise sample-specific barcodes, and wherein the method further comprises pooling the amplification products from a plurality of samples prior to high-throughput sequencing and sequencing the pool of amplification products together in a single run during the high-throughput sequencing.

In some exemplary embodiments, the amount of nucleic acids is determined by using for example, tracer nucleic acids, or internal calibration nucleic acids. The terms “tracer nucleic acids,” or “internal calibration nucleic acids” are used interchangeably and refer to a composition of nucleic acids for which one or more of the following is known advance-length, sequence, nucleotide composition, quantity, or biological origin. The tracer can be added to a biological sample derived from a human subject to help estimate the amount of total RNA or cfDNA in said sample. It can also be added to reaction mixtures other than the biological sample itself.

### Cutoff Threshold for Determining Transplant Rejection

In some exemplary embodiments, the cutoff threshold is an estimate percentage of donor-derived RNA out of total RNA or a function thereof. In some exemplary embodiments, the cutoff threshold is 1.0%, 1.1%, 1.2%, 1.3%, 1.4%, 1.5%, 1.6%, 1.7%, 1.8%, 1.9%, or 2.0% RNA (e.g. mRNA or miRNA). In some exemplary embodiments, the cutoff threshold is 1.0%, 1.1%, 1.2%, 1.3%, 1.4%, 1.5%, 1.6%, 1.7%, 1.8%, 1.9%, or 2.0% cell-free DNA or combination of cell-free DNA and RNA. In some exemplary embodiments, the cutoff threshold is adjusted according to the type of organs transplanted. In some exemplary embodiments, the cutoff threshold is adjusted according to the number of organs transplanted.

In some exemplary embodiments, the cutoff threshold is an estimate percentage of donor-derived RNA out of total RNA or a function thereof. In some exemplary embodiments, the cutoff threshold is 1.0%, 1.1%, 1.2%, 1.3%, 1.4%, 1.5%, 1.6%, 1.7%, 1.8%, 1.9%, or 2.0% RNA. In some exemplary embodiments, the cutoff threshold is 1.0%, 1.1%, 1.2%, 1.3%, 1.4%, 1.5%, 1.6%, 1.7%, 1.8%, 1.9%, or 2.0% cell-free DNA or combination of cell-free DNA and RNA In some exemplary embodiments, the cutoff threshold is adjusted according to the type of organs transplanted. In some exemplary embodiments, the cutoff threshold is adjusted according to the number of organs transplanted.

In some exemplary embodiments, the cutoff threshold is an estimate percentage of donor-derived mRNA out of total mRNA or a function thereof. In some exemplary embodiments, the cutoff threshold is 1.0%, 1.1%, 1.2%, 1.3%, 1.4%, 1.5%, 1.6%, 1.7%, 1.8%, 1.9%, or 2.0% RNA. In some exemplary embodiments, the cutoff threshold is 1.0%, 1.1%, 1.2%, 1.3%, 1.4%, 1.5%, 1.6%, 1.7%, 1.8%, 1.9%, or 2.0% cell-free DNA or combination of cell-free DNA and mRNA In some exemplary embodiments, the cutoff threshold is adjusted according to the type of organs transplanted. In some exemplary embodiments, the cutoff threshold is adjusted according to the number of organs transplanted.

In some exemplary embodiments, the cutoff threshold is an estimate percentage of donor-derived preselected mRNA targets out of total mRNA or a function thereof. In some exemplary embodiments, the cutoff threshold is 1.0%, 1.1%, 1.2%, 1.3%, 1.4%, 1.5%, 1.6%, 1.7%, 1.8%, 1.9%, or 2.0% RNA. In some exemplary embodiments, the cutoff threshold is 1.0%, 1.1%, 1.2%, 1.3%, 1.4%, 1.5%, 1.6%, 1.7%, 1.8%, 1.9%, or 2.0% cell-free DNA or combination of cell-free DNA and the preselected mRNA targets. In some exemplary embodiments, the cutoff threshold is adjusted according to the type of organs transplanted. In some exemplary embodiments, the cutoff threshold is adjusted according to the number of organs transplanted

In some exemplary embodiments, the cutoff threshold is proportional to an absolute donor-derived RNA concentration. In some exemplary embodiments, the cutoff threshold is a copy number of donor-derived RNA or a function thereof. In some exemplary embodiments, the cutoff threshold is expressed as quantity or absolute quantity of RNA. In some exemplary embodiments, the cutoff threshold is expressed as quantity or absolute quantity of RNA per volume unit of the blood sample. In some exemplary embodiments, the cutoff threshold is expressed as quantity or absolute quantity of RNA per volume unit of the blood sample multiplied by body mass, BMI, or blood volume of the transplant recipient.

In some exemplary embodiments, the cutoff threshold is proportional to an absolute donor-derived RNA concentration. In some exemplary embodiments, the cutoff threshold is a copy number of donor-derived RNA or a function thereof. In some exemplary embodiments, the cutoff threshold is expressed as quantity or absolute quantity of RNA. In some exemplary embodiments, the cutoff threshold is expressed as quantity or absolute quantity of RNA per volume unit of the blood sample. In some exemplary embodiments, the cutoff threshold is expressed as quantity or absolute quantity of RNA per volume unit of the blood sample multiplied by body mass, BMI, or blood volume of the transplant recipient.

In some exemplary embodiments, the method further comprises repeating steps (a)-(d) longitudinally for the same transplant recipient, and determining a longitudinal change in the amount of RNA or a function thereof and a longitudinal change in the amount of donor-derived RNA or a function thereof.

### Definitions

As used herein the term “single nucleotide polymorphism (SNP)” refers to a single nucleotide that may differ between the genomes of two members of the same species. The usage of the term does not imply any limit on the frequency with which each variant occurs.

In some exemplary embodiments, for example, sequence refers to a DNA or RNA sequence or a genetic sequence. It may refer to the primary, physical structure of the DNA or RNA molecule or strand in an individual. It may refer to the sequence of nucleotides found in that DNA or RNA molecule, or the complementary strand to the DNA or RNA molecule. It may refer to the information contained in the DNA or RNA molecule as its representation in silico.

In some exemplary embodiments, for example, locus refers to a particular region of interest on the DNA or RNA of an individual and includes without limitation one or more SNPs, the site of a possible insertion or deletion, or the site of some other relevant genetic variation. Disease-linked SNPs may also refer to disease-linked loci.

In some exemplary embodiments, for example, polymorphic allele, also “polymorphic locus,” refers to an allele or locus where the genotype varies between individuals within a given species. Some examples of polymorphic alleles include single nucleotide polymorphisms (SNPs), short tandem repeats, deletions, duplications, and inversions.

In some exemplary embodiments, for example, allele refers to the nucleotides or nucleotide sequence occupying a particular locus.

In some exemplary embodiments, for example, genetic data also “genotypic data” refers to the data describing aspects of the genome of one or more individuals. It may refer to one or a set of loci, partial or entire sequences, partial or entire chromosomes, or the entire genome. It may refer to the identity of one or a plurality of nucleotides; it may refer to a set of sequential nucleotides, or nucleotides from different locations in the genome, or a combination thereof. Genotypic data is typically in silico, however, it is also possible to consider physical nucleotides in a sequence as chemically encoded genetic data. Genotypic Data may be said to be “on,” “of,” “at,” “from” or “on” the individual(s). Genotypic Data may refer to output measurements from a genotyping platform where those measurements are made on genetic material.

In some exemplary embodiments, for example, genetic material also “genetic sample” refers to physical matter, such as tissue or blood, from one or more individuals comprising nucleic acids (e.g., comprising DNA or RNA).

In some exemplary embodiments, for example, allelic data refers to a set of genotypic data concerning a set of one or more alleles. It may refer to the phased, haplotypic data. It may refer to SNP identities, and it may refer to the sequence data of the nucleic acid, including insertions, deletions, repeats and mutations.

In some exemplary embodiments, for example, allelic state refers to the actual state of the genes in a set of one or more alleles. It may refer to the actual state of the genes described by the allelic data.

In some exemplary embodiments, for example, allelic ratio or allele ratio, refers to the ratio between the amount of each allele at a locus that is present in a sample or in an individual. When the sample was measured by sequencing, the allelic ratio may refer to the ratio of sequence reads that map to each allele at the locus. When the sample was measured by an intensity based measurement method, the allele ratio may refer to the ratio of the amounts of each allele present at that locus as estimated by the measurement method.

In some exemplary embodiments, for example, allele count refers to the number of sequences that map to a particular locus, and if that locus is polymorphic, it refers to the number of sequences that map to each of the alleles. If each allele is counted in a binary fashion, then the allele count will be whole number. If the alleles are counted probabilistically, then the allele count can be a fractional number.

In some exemplary embodiments, for example, primer, also “PCR probe” refers to a single DNA molecule (a DNA oligomer) or a collection of DNA molecules (DNA oligomers) where the DNA molecules are identical, or nearly so, and where the primer contains a region that is designed to hybridize to a targeted polymorphic locus, and contain a priming sequence designed to allow amplification such as PCR amplification. A primer may also contain a molecular barcode. A primer may contain a random region that differs for each individual molecule.

In some exemplary embodiments, for example, hybrid capture probe refers to any nucleic acid sequence, possibly modified, that is generated by various methods such as PCR or direct synthesis and intended to be complementary to one strand of a specific target DNA or RNA sequence in a sample. The exogenous hybrid capture probes may be added to a prepared sample and hybridized through a denaturation-reannealing process to form duplexes of exogenous-endogenous fragments. These duplexes may then be physically separated from the sample by various means.

In some exemplary embodiments, for example, sequence read refers to data representing a sequence of nucleotide bases that were measured using a clonal sequencing method. Clonal sequencing may produce sequence data representing single, or clones, or clusters of one original DNA or RNA molecule. A sequence read may also have associated quality score at each base position of the sequence indicating the probability that nucleotide has been called correctly.

In some exemplary embodiments, for example, mapping a sequence read is the process of determining a sequence read's location of origin in the genome sequence of a particular organism. The location of origin of sequence reads is based on similarity of nucleotide sequence of the read and the genome sequence.

In some exemplary embodiments, for example, DNA or RNA of donor origin refers to DNA or RNA that was originally part of a cell whose genotype was essentially equivalent to that of the transplant donor. The donor can be a human or a non-human mammalian (e.g., pig).

In some exemplary embodiments, for example, DNA or RNA of recipient origin refers to DNA or RNA that was originally part of a cell whose genotype was essentially equivalent to that of the transplant recipient.

In some exemplary embodiments, RNA may refer to messenger RNA (mRNA), small non-coding RNA (sncRNA), transfer RNA (tRNA), or a non-protein coding RNA from cells. In some exemplary embodiments, sncRNA comprises micro RNA (miRNA), piwi-interacting RNA (piRNA), small nucleolar RNA (snoRNA), small nuclear RNA (snRNA), or miscellaneous

RNA (miscRNA). In some exemplary embodiments, the RNA is cell-free RNA. In some exemplary embodiments, the cell-free RNA is derived from exosomes or microvesicles.

In some exemplary embodiments, amplification of RNA comprises reverse transcription of RNA to produce complementary DNA (cDNA) followed by amplification of cDNA by amplification methods disclosed elsewhere herein.

In some exemplary embodiments, for example, transplant recipient plasma refers to the plasma portion of the blood from a female from a patient who has received an allograft or xenograft, e.g., an organ transplant recipient.

In some exemplary embodiments, for example, preferential enrichment of DNA or RNA that corresponds to a locus, or preferential enrichment of DNA or RNA at a locus, refers to any technique that results in the percentage of molecules of DNA or RNA in a post-enrichment DNA or RNA mixture that correspond to the locus being higher than the percentage of molecules of DNA or RNA in the pre-enrichment DNA or RNA mixture that correspond to the locus. The technique may involve selective amplification of DNA or RNA molecules that correspond to a locus. The technique may involve removing DNA or RNA molecules that do not correspond to the locus. The technique may involve a combination of methods. The degree of enrichment is defined as the percentage of molecules of DNA or RNA in the post-enrichment mixture that correspond to the locus divided by the percentage of molecules of DNA or RNA in the pre-enrichment mixture that correspond to the locus. Preferential enrichment may be carried out at a plurality of loci. In some exemplary embodiments of the present disclosure, the degree of enrichment is greater than 20. In some exemplary embodiments of the present disclosure, the degree of enrichment is greater than 200. In some exemplary embodiments of the present disclosure, the degree of enrichment is greater than 2,000. When preferential enrichment is carried out at a plurality of loci, the degree of enrichment may refer to the average degree of enrichment of all of the loci in the set of loci.

In some exemplary embodiments, for example, amplification refers to a technique that increases the number of copies of a molecule of RNA and/or DNA.

In some exemplary embodiments, for example, selective amplification may refer to a technique that increases the number of copies of a particular molecule of RNA and/or DNA, or molecules of RNA and/or DNA that correspond to a particular region of RNA and/or DNA. It may also refer to a technique that increases the number of copies of a particular targeted molecule of RNA and/or DNA, or targeted region of RNA and/or DNA more than it increases non-targeted molecules or regions of RNA and/or DNA. Selective amplification may be a method of preferential enrichment.

In some exemplary embodiments, for example, universal priming sequence refers to a DNA sequence that may be appended to a population of target nucleic acid molecules, for example by ligation, PCR, or ligation mediated PCR. Once added to the population of target molecules, primers specific to the universal priming sequences can be used to amplify the target population using a single pair of amplification primers. Universal priming sequences need not be related to the target sequences.

In some exemplary embodiments, for example, universal adapters, or ‘ligation adaptors’ or ‘library tags’ are DNA molecules containing a universal priming sequence that can be covalently linked to the 5-prime and 3-prime end of a population of target double stranded DNA molecules. The addition of the adapters provides universal priming sequences to the 5-prime and 3-prime end of the target population from which PCR amplification can take place, amplifying all molecules from the target population, using a single pair of amplification primers.

In some exemplary embodiments, for example, targeting refers to a method used to selectively amplify or otherwise preferentially enrich those molecules of DNA or RNA that correspond to a set of loci in a mixture of DNA or RNA.

### Analysis of Donor-Derived RNA for Monitoring Transplant Rejection

“Acute rejection or AR” is the rejection by the immune system of a tissue transplant recipient when the transplanted tissue is immunologically foreign. Acute rejection is characterized by infiltration of the transplanted tissue by immune cells of the recipient, which carry out their effector function and destroy the transplanted tissue. The onset of acute rejection is rapid and generally occurs in humans within a few weeks after transplant surgery. Generally, acute rejection can be inhibited or suppressed with immunosuppressive drugs such as rapamycin, cyclosporin A, anti-CD40L monoclonal antibody and the like.

“Chronic transplant rejection or injury” or “CAI” generally occurs in humans within several months to years after engraftment, even in the presence of successful immunosuppression of acute rejection. Fibrosis is a common factor in chronic rejection of all types of organ transplants. Chronic rejection can typically be described by a range of specific disorders that are characteristic of the particular organ. For example, in lung transplants, such disorders include fibroproliferative destruction of the airway (bronchiolitis obliterans); in heart transplants or transplants of cardiac tissue, such as valve replacements, such disorders include fibrotic atherosclerosis; in kidney transplants, such disorders include, obstructive nephropathy, nephrosclerorsis, tubulointerstitial nephropathy; and in liver transplants, such disorders include disappearing bile duct syndrome. Chronic rejection can also be characterized by ischemic insult, denervation of the transplanted tissue, hyperlipidemia and hypertension associated with immunosuppressive drugs.

The term “transplant rejection” encompasses both acute and chronic transplant rejection. The term “transplant injury” refers to all manners of graft dysfunction, irrespective of pathological diagnosis. The term “organ injury” refers to biomarkers that track with poor function of the organ, irrespective of the organ being native or a transplant, and irrespective of the etiology.

In one aspect, the present invention relates to a method of quantifying the amount of donor-derived RNA (dd-RNA) in a sample obtained from a transplant recipient, comprising: extracting RNA from the sample obtained from the transplant recipient, wherein the RNA comprises donor-derived RNA and recipient-derived RNA; performing targeted amplification at 10-50,000 target loci in a single reaction volume using 10-50,000 primer pairs, wherein the target loci comprise polymorphic loci and non-polymorphic loci, and quantifying the amount of donor-derived RNA and/or cell-free DNA in the amplification products. In some exemplary embodiments, the donor-derived RNA may be donor-derived mRNA or donor-derived miRNA.

In another aspect, the present invention relates to a method of quantifying the amount of donor-derived RNA (dd-RNA) in a blood, serum or plasma sample obtained from a transplant recipient, comprising: extracting RNA from the sample from the transplant recipient, wherein the RNA comprises donor-derived RNA and recipient-derived RNA, and utilizing Cas9/Cas12a to deplete contaminating or overabundant species in whole blood or hemolysis tainted blood, serum or plasma samples to increase the sensitivity of detecting the target loci; performing targeted amplification at 10-50,000 target loci in a single reaction volume using 10-50,000 primer pairs, wherein the target loci comprise polymorphic loci and non-polymorphic loci; and quantifying the amount of donor-derived cell-free DNA in the amplification products. In some exemplary embodiments, the contaminating or overabundant species comprise hemoglobin mRNA and miR-451, miR-144, and miR-486.

In another aspect, the present invention relates to a method of quantifying the amount of donor-derived RNA (dd-RNA) in a sample obtained from a transplant recipient, comprising: extracting RNA from the sample from the transplant recipient, wherein the RNA comprises donor-derived RNA and recipient-derived RNA; performing targeted amplification at 10-50,000 target loci in a single reaction volume using 10-50,000 primer pairs, wherein the target loci comprise polymorphic loci and non-polymorphic loci; depleting adaptor dimers, primer dimers, and unwanted ligation products from the composition of amplified nucleic acids comprising target loci, thereby increasing the fraction of desired reads that map to a target loci of interest per sample and the sample throughput per sequencing run and quantifying the amount of donor-derived cell-free DNA in the amplification products.

In another aspect, the present invention relates to a method of detecting donor-derived RNA in a blood sample of a transplant recipient, comprising: extracting RNA from the blood sample of the transplant recipient, wherein the RNA comprises donor-derived RNA and recipient-derived RNA; performing targeted amplification at 100-50,000 target loci in a single reaction volume using 100-50,000 primer pairs, wherein the target loci comprise polymorphic loci and non-polymorphic loci; sequencing the amplification products by high-throughput sequencing; and quantifying the amount of donor-derived RNA. In some exemplary embodiments, the method comprises targeted amplification of about 100 to about 1000, about 200-2000, about 1000-10000, about 2000-10000, about 2000-50000 target loci.

In some exemplary embodiments, the method comprises universal amplification of the extracted RNA. In some exemplary embodiments, the universal amplification preferentially amplifies donor-derived RNA over recipient-derived RNA.

In some exemplary embodiments, the transplant recipient is a mammal. In some exemplary embodiments, the transplant recipient is a human.

In some exemplary embodiments, the transplant recipient has received a transplant selected from organ transplant, tissue transplant, cell transplant, and fluid transplant. In some exemplary embodiments, the transplant recipient has received a transplant selected from kidney transplant, liver transplant, pancreas transplant, intestinal transplant, heart transplant, lung transplant, heart/lung transplant, stomach transplant, testis transplant, penis transplant, ovary transplant, uterus transplant, thymus transplant, face transplant, hand transplant, leg transplant, bone transplant, bone marrow transplant, cornea transplant, skin transplant, pancreas islet cell transplant, heart valve transplant, blood vessel transplant, and blood transfusion. In some exemplary embodiments, the transplant recipient has received SPK transplant.

In some exemplary embodiments, the quantifying step comprises determining the percentage of donor-derived RNA out of the total of donor-derived RNA and recipient-derived RNA in the blood sample. In some exemplary embodiments, the quantifying step comprises determining the number of copies of donor-derived RNA per volume unit of the blood sample.

In some exemplary embodiments, the method further comprises detecting the occurrence or likely occurrence of active rejection of transplantation using the quantified amount of donor-derived RNA. In some exemplary embodiments, the method is performed without prior knowledge of donor genotypes.

In some exemplary embodiments, each primer pair is designed to amplify a target sequence of about 50-100 bp. In some exemplary embodiments, each primer pair is designed to amplify a target sequence of no more than 75 bp. In some exemplary embodiments, each primer pair is designed to amplify a target sequence of about 60-75 bp. In some exemplary embodiments, each primer pair is designed to amplify a target sequence of about 65 bp.

In some exemplary embodiments, the targeted amplification comprises amplifying at least 1,000 polymorphic loci in a single reaction volume. In some exemplary embodiments, the targeted amplification comprises amplifying at least 2,000 polymorphic loci in a single reaction volume. In some exemplary embodiments, the targeted amplification comprises amplifying at least 5,000 polymorphic loci in a single reaction volume. In some exemplary embodiments, the targeted amplification comprises amplifying at least 10,000 polymorphic loci in a single reaction volume. In some exemplary embodiments, the targeted amplification comprises amplifying from about 100 to about 50000 polymorphic loci in a single reaction volume. In some exemplary embodiments, the targeted amplification comprises amplifying from about 1000 to about 50000 polymorphic loci in a single reaction volume. In some exemplary embodiments, the targeted amplification comprises amplifying from about 5000 to about 50000 polymorphic loci in a single reaction volume.

In some exemplary embodiments, method further comprises measuring an amount of one or more alleles at the target loci that are polymorphic loci. In some exemplary embodiments, the polymorphic loci and the non-polymorphic loci are amplified in a single reaction.

In some exemplary embodiments, the quantifying step comprises detecting the amplified target loci using a microarray. In some exemplary embodiments, the quantifying step does not comprise using a microarray.

In some exemplary embodiments, the targeted amplification comprises simultaneously amplifying 50-50,000 target loci in a single reaction volume using (i) at least 50-50,000 different primer pairs, or (ii) at least 50-50,000 target-specific primers and a universal or tag-specific primer 50-50,000 primer pairs.

In a further aspect, the present invention relates to an exemplary method of determining the likelihood of transplant rejection within a transplant recipient, the method comprising: extracting RNA from the blood sample of the transplant recipient, wherein the RNA comprises donor-derived RNA and recipient-derived RNA; performing universal amplification of the extracted RNA; performing targeted amplification at 50-50,000 target loci in a single reaction volume using 50-50,000 primer pairs, wherein the target loci comprise polymorphic loci and non-polymorphic loci; sequencing the amplification products by high-throughput sequencing; and quantifying the amount of donor-derived RNA in the blood sample, wherein a greater amount of dd-RNA indicates a greater likelihood of transplant rejection.

In a further aspect, the present invention relates to an exemplary method of diagnosing a transplant within a transplant recipient as undergoing acute rejection, the method comprising: extracting RNA from the blood sample of the transplant recipient, wherein the RNA comprises donor-derived RNA and recipient-derived RNA; performing universal amplification of the extracted RNA; performing targeted amplification at 50-50,000 target loci in a single reaction volume using 50-50,000 primer pairs, wherein the target loci comprise polymorphic loci and non-polymorphic loci; sequencing the amplification products by high-throughput sequencing; and quantifying the amount of donor-derived RNA in the blood sample, wherein an amount of dd-RNA of greater than 1% (or 1.1%, or 1.2%, or 1.3%, or 1.4%, or 1.5%, or 1.6%, or 1.7%, or 1.8%, or 1.9%, or 2.0%) indicates that the transplant is undergoing acute rejection.

In some exemplary embodiments, the transplant rejection is antibody mediated transplant rejection. In some exemplary embodiments, the transplant rejection is T cell mediated transplant rejection.

In some exemplary embodiments, an amount of dd-RNA of less than 1% (or 0.9%, or 0.8%, or 0.7%, or 0.6%, or 0.5%) indicates that the transplant is either undergoing borderline rejection, undergoing other injury, or stable.

In a further aspect, the present invention relates to an exemplary method of monitoring immunosuppressive therapy in a subject, the method comprising: extracting RNA from the blood sample of the transplant recipient, wherein the RNA comprises donor-derived RNA and recipient-derived RNA; performing universal amplification of the extracted RNA; performing targeted amplification at 500-50,000 target loci in a single reaction volume using 500-50,000 primer pairs, wherein the target loci comprise polymorphic loci and non-polymorphic loci; sequencing the amplification products by high-throughput sequencing; and quantifying the amount of donor-derived RNA in the blood sample, wherein a change in levels of dd-RNA over a time interval is indicative of transplant status.

In some exemplary embodiments, the method further comprising adjusting immunosuppressive therapy based on the levels of dd-RNA over the time interval.

In some exemplary embodiments, for example, an increase in the levels of dd-RNA is indicative of transplant rejection and a need for adjusting immunosuppressive therapy. In some exemplary embodiments, no change or a decrease in the levels of dd-RNA indicates transplant tolerance or stability, and a need for adjusting immunosuppressive therapy.

In some exemplary embodiments, for example, an amount of dd-RNA of greater than 1% (or 1.1%, or 1.2%, or 1.3%, or 1.4%, or 1.5%, or 1.6%, or 1.7%, or 1.8%, or 1.9%, or 2.0%) indicates that the transplant is undergoing acute rejection. In some exemplary embodiments, the transplant rejection is antibody mediated transplant rejection. In some exemplary embodiments, the transplant rejection is T cell mediated transplant rejection.

In some exemplary embodiments, an amount of dd-RNA of less than 1% (or 0.9%, or 0.8%, or 0.7%, or 0.6%, or 0.5%) indicates that the transplant is either undergoing borderline rejection, undergoing other injury, or stable.

In some embodiments, the method does not comprise genotyping the transplant donor and/or the transplant recipient.

In some embodiments, the method further comprises measuring an amount of one or more alleles at the target loci that are polymorphic loci.

In some embodiments, the transplant recipient is a human. In some embodiments, the transplant recipient has received a transplant selected from a kidney transplant, liver transplant, pancreas transplant, islet cell transplant, intestinal transplant, heart transplant, lung transplant, bone marrow transplant, heart valve transplant, or a skin transplant. In some embodiments, the transplant recipient has received SPK transplant.

In some embodiments, the extracting step comprises size selection to enrich for donor-derived RNA and reduce the amount of recipient-derived RNA disposed from bursting blood cells.

In some embodiments, the universal amplification step preferentially amplifies donor-derived RNA over recipient-derived RNA that are disposed from bursting blood cells.

In some embodiments, the method comprises longitudinally collecting a plurality of blood samples from the transplant recipient after transplantation, and repeating steps (a) to (e) for each blood sample collected. In some embodiments, the method comprises collecting and analyzing blood samples from the transplant recipient for a time period of about three months, or about six months, or about twelve months, or about eighteen months, or about twenty-four months, etc. In some embodiments, the method comprises collecting blood samples from the transplant recipient at an interval of about one week, or about two weeks, or about three weeks, or about one month, or about two months, or about three months, etc.

In some embodiments, the method has a sensitivity of at least 80%, or at least 85%, or at least 90%, or at least 95%, or at least 98% in identifying acute rejection (AR) over non-AR with a cutoff threshold of 1% dd-RNA and a confidence interval of 95%.

In some embodiments, the method has a specificity of at least 60%, or at least 65%, or at least 70%, or at least 75%, or at least 80%, or at least 85%, or at least 90% in identifying AR over non-AR with a cutoff threshold of 1% dd-RNA and a confidence interval of 95%.

In some embodiments, the method has an area under the curve (AUC) of at least 0.8, or 0.85, or at least 0.9, or at least 0.95 in identifying AR over non-AR with a cutoff threshold of 1% dd-RNA and a confidence interval of 95%.

In some exemplary embodiments, for example, the method has a sensitivity of at least 80%, or at least 85%, or at least 90%, or at least 95%, or at least 98% in identifying AR over normal, stable allografts (STA) with a cutoff threshold of 1% dd-RNA and a confidence interval of 95%.

In some exemplary embodiments, for example, the method has a specificity of at least 80%, or at least 85%, or at least 90%, or at least 95%, or at least 98% in identifying AR over STA with a cutoff threshold of 1% dd-RNA and a confidence interval of 95%.

In some exemplary embodiments, for example, the method has an AUC of at least 0.8, or 0.85, or at least 0.9, or at least 0.95, or at least 0.98, or at least 0.99 in identifying AR over STA with a cutoff threshold of 1% dd-RNA and a confidence interval of 95%.

In some exemplary embodiments, for example, the method has a sensitivity as determined by a limit of blank (LoB) of 0.5% or less, and a limit of detection (LoD) of 0.5% or less. In some exemplary embodiments, LoB is 0.23% or less and LoD is 0.29% or less. In some exemplary embodiments, the sensitivity is further determined by a limit of quantitation (LoQ). In some exemplary embodiments, LoQ is 10 times greater than the LoD; LoQ may be 5 times greater than the LoD; LoQ may be 1.5 times greater than the LoD; LoQ may be 1.2 times greater than the LoD; LoQ may be 1.1 times greater than the LoD; or LoQ may be equal to or greater than the LoD. In some exemplary embodiments, LoB is equal to or less than 0.04%, LoD is equal to or less than 0.05%, and/or LoQ is equal to the LoD.

In some exemplary embodiments, for example, the method has an accuracy as determined by evaluating a linearity value obtained from linear regression analysis of measured donor fractions as a function of the corresponding attempted spike levels, wherein the linearity value is a R2 value, wherein the R2 value is from about 0.98 to about 1.0. In some exemplary embodiments, the R2 value is 0.999. In some exemplary embodiments, for example, the method has an accuracy as determined by using linear regression on measured donor fractions as a function of the corresponding attempted spike levels to calculate a slope value and an intercept value, wherein the slope value is from about 0.9 to about 1.2 and the intercept value is from about-0.0001 to about 0.01. In some exemplary embodiments, the slope value is approximately 1, and the intercept value is approximately 0.

In some exemplary embodiments, for example, the method has a precision as determined by calculating a coefficient of variation (CV), wherein the CV is less than about 10.0%. CV is less than about 6%. In some exemplary embodiments, the CV is less than about 4%. In some exemplary embodiments, the CV is less than about 2%. In some exemplary embodiments, the CV is less than about 1%.

In some exemplary embodiments, for example, the AR is antibody-mediated rejection (ABMR). In some exemplary embodiments, the AR is T-cell-mediated rejection (TCMR).

Further disclosed herein are exemplary methods for detection of transplant donor-derived RNA (dd-RNA) in a sample from a transplant recipient. In some exemplary embodiments, in the exemplary methods disclosed herein, the transplant recipient is a mammal. In some exemplary embodiments, the transplant recipient is a human. In some exemplary embodiments, the transplant recipient has received a transplant selected from a kidney transplant, liver transplant, pancreas transplant, islet cell transplant, intestinal transplant, heart transplant, lung transplant, bone marrow transplant, heart valve transplant, or a skin transplant. In some exemplary embodiments, the transplant recipient has received SPK transplant. In some exemplary embodiments, the method may be performed on transplant recipients the day of or after transplant surgery, up to a year following transplant surgery.

In some exemplary embodiments, disclosed herein is a method of amplifying target loci of donor-derived RNA (dd-RNA) from a blood sample of a transplant recipient, the method comprising: a) extracting RNA from the blood sample of the transplant recipient, wherein the RNA comprises RNA derived from both the transplanted cells and from the transplant recipient, b) enriching the extracted RNA at target loci, wherein the target loci comprise 50 to 5000 target loci comprising polymorphic loci and non-polymorphic loci; and c) amplifying the target loci.

In some exemplary embodiments, disclosed herein is a method of detecting donor-derived RNA (dd-RNA) in a sample from a transplant recipient, the method comprising: a) extracting RNA from the sample of the transplant recipient, wherein the RNA comprises RNA derived from both the transplanted cells and from the transplant recipient, b) enriching the extracted RNA at target loci, wherein the target loci comprise 50 to 5000 target loci comprising polymorphic loci and non-polymorphic loci; c) amplifying the target loci; d) contacting the amplified target loci with probes that specifically hybridize to target loci; and e) detecting binding of the target loci with the probes, thereby detecting RNA in the blood sample. In some exemplary embodiments, the probes are labelled with a detectable marker.

In some exemplary embodiments, disclosed herein is a method of determining the likelihood of transplant rejection within a transplant recipient, the method comprising: a) extracting RNA from the sample of the transplant recipient, wherein the RNA comprises RNA derived from both the transplanted cells and from the transplant recipient, b) enriching the extracted RNA at target loci, wherein the target loci comprise 50 to 5000 target loci comprising polymorphic loci and non-polymorphic loci; c) amplifying the target loci; and d) measuring an amount of transplant RNA and an amount of recipient RNA in the recipient sample; wherein a greater amount of dd-RNA indicates a greater likelihood of transplant rejection.

In some exemplary embodiments, disclosed herein is a method of diagnosing a transplant within a transplant recipient as undergoing acute rejection, the method comprising: a) extracting RNA from the blood sample of the transplant recipient, wherein the RNA comprises RNA derived from both the transplanted cells and from the transplant recipient, b) enriching the extracted RNA at target loci, wherein the target loci comprise 50 to 5000 target loci comprising polymorphic loci and non-polymorphic loci; c) amplifying the target loci; and d) measuring an amount of transplant RNA and an amount of recipient RNA in the recipient sample; wherein an amount of dd-RNA of greater than 1% (or 1.1%, or 1.2%, or 1.3%, or 1.4%, or 1.5%, or 1.6%, or 1.7%, or 1.8%, or 1.9%, or 2.0%) indicates that the transplant is undergoing acute rejection.

In some exemplary embodiments, in the methods disclosed herein, the transplant rejection is antibody mediated transplant rejection. In some exemplary embodiments, the transplant rejection is T cell mediated transplant rejection. In some exemplary embodiments, an amount of dd-RNA of less than 1% (or 0.9%, or 0.8%, or 0.7%, or 0.6%, or 0.5%) indicates that the transplant is either undergoing borderline rejection, undergoing other injury, or stable.

In some exemplary embodiments In some exemplary embodiments, disclosed herein is a method of monitoring immunosuppressive therapy in a subject, the method comprising a) extracting RNA from the blood sample of the transplant recipient, wherein the RNA comprises RNA derived from both the transplanted cells and from the transplant recipient, b) enriching the extracted RNA at target loci, wherein the target loci comprise 50 to 5000 target loci comprising polymorphic loci and non-polymorphic loci; c) amplifying the target loci; and d) measuring an amount of transplant DNA and an amount of recipient DNA in the recipient blood sample; wherein a change in levels of dd-RNA over a time interval is indicative of transplant status. In some exemplary embodiments. In some exemplary embodiments, the method further comprises adjusting immunosuppressive therapy based on the levels of dd-RNA over the time interval. In some exemplary embodiments. In some exemplary embodiments, an increase in the levels of dd-RNA are indicative of transplant rejection and a need for adjusting immunosuppressive therapy. In some exemplary embodiments. In some exemplary embodiments, a change or a decrease in the levels of dd-RNA indicates transplant tolerance or stability, and a need for adjusting immunosuppressive therapy.

In some exemplary embodiments, in the methods disclosed herein, the target loci that are amplified in amplicons of about 50-100 bp in length, or about 60-80 bp in length. In some exemplary embodiments, the amplicons are about 65 bp in length.

In some exemplary embodiments, the methods disclosed herein further comprise measuring an amount of transplant RNA and an amount of recipient RNA in the recipient blood sample.

In some exemplary embodiments, the methods disclosed herein do not comprise genotyping the transplant donor and the transplant recipient.

In some exemplary embodiments, the methods disclosed herein further comprise detecting the amplified target loci using a microarray.

In some exemplary embodiments, in the methods disclosed herein, the polymorphic loci and the non-polymorphic loci are amplified in a single reaction.

In some exemplary embodiments, in the methods disclosed herein, the RNA is preferentially enriched at the target loci.

In some exemplary embodiments, preferentially enriching the RNA in the sample at the plurality of target loci includes obtaining a plurality of pre-circularized probes where each probe targets one of the target loci, and where the 3′ and 5′ end of the probes are designed to hybridize to a region of RNA that is separated from the polymorphic site of the locus by a small number of bases, where the small number is 1, 2, 3, 4, 5, 6, 7, 8, 9, 10, 11, 12, 13, 14, 15, 16, 17, 18, 19, 20, 21 to 25, 26 to 30, 31 to 60, or a combination thereof, hybridizing the pre-circularized probes to RNA converted to cDNA from the sample, filling the gap between the hybridized probe ends using DNA polymerase, circularizing the pre-circularized probe, and amplifying the circularized probe.

In some exemplary embodiments, preferentially enriching the RNA at the plurality of polymorphic loci includes obtaining a plurality of ligation-mediated PCR probes where each PCR probe targets one of the polymorphic loci, and where the upstream and downstream PCR probes are designed to hybridize to a region of cDNA derived from the extracted RNA, on one strand of the cDNA, that is separated from the polymorphic site of the locus by a small number of bases, where the small number is 1, 2, 3, 4, 5, 6, 7, 8, 9, 10, 11, 12, 13, 14, 15, 16, 17, 18, 19, 20, 21 to 25, 26 to 30, 31 to 60, or a combination thereof, hybridizing the ligation-mediated PCR probes to the cDNA from the first sample, filling the gap between the ligation-mediated PCR probe ends using DNA polymerase, ligating the ligation-mediated PCR probes, and amplifying the ligated ligation-mediated PCR probes.

In some exemplary embodiments, preferentially enriching the RNA at the plurality of target loci includes obtaining a plurality of hybrid capture probes that target specific loci, hybridizing the hybrid capture probes to the RNA in the sample and physically removing some or all of the unhybridized RNA from the first sample of RNA.

In some exemplary embodiments, the hybrid capture probes are designed to hybridize to a region that is flanking but not overlapping the polymorphic site. In some exemplary embodiments In some exemplary embodiments, the hybrid capture probes are designed to hybridize to a region that is flanking but not overlapping the polymorphic site, and where the length of the flanking capture probe may be selected from the group consisting of less than about 120 bases, less than about 110 bases, less than about 100 bases, less than about 90 bases, less than about 80 bases, less than about 70 bases, less than about 60 bases, less than about 50 bases, less than about 40 bases, less than about 30 bases, and less than about 25 bases. In some exemplary embodiments, the hybrid capture probes are designed to hybridize to a region that overlaps the polymorphic site, and where the plurality of hybrid capture probes comprise at least two hybrid capture probes for each polymorphic loci, and where each hybrid capture probe is designed to be complementary to a different allele at that polymorphic locus.

In some exemplary embodiments, preferentially enriching the RNA or cDNA derived from the RNA at a plurality of polymorphic loci includes obtaining a plurality of inner forward primers where each primer targets one of the polymorphic loci, and where the 3′ end of the inner forward primers are designed to hybridize to a region of RNA or cDNA derived from the RNA upstream from the polymorphic site, and separated from the polymorphic site by a small number of bases, where the small number is selected from the group consisting of 1, 2, 3, 4, 5, 6 to 10, 11 to 15, 16 to 20, 21 to 25, 26 to 30, or 31 to 60 base pairs, optionally obtaining a plurality of inner reverse primers where each primer targets one of the polymorphic loci, and where the 3′ end of the inner reverse primers are designed to hybridize to a region of RNA or cDNA derived from the RNA upstream from the polymorphic site, and separated from the polymorphic site by a small number of bases, where the small number is selected from the group consisting of 1, 2, 3, 4, 5, 6 to 10, 11 to 15, 16 to 20, 21 to 25, 26 to 30, or 31 to 60 base pairs, hybridizing the inner primers to the RNA or cDNA derived from the RNA, and amplifying the cDNA using the polymerase chain reaction to form amplicons.

In some exemplary embodiments, the method also includes obtaining a plurality of outer forward primers where each primer targets one of the polymorphic loci, and where the outer forward primers are designed to hybridize to the region of RNA or cDNA derived from the RNA upstream from the inner forward primer, optionally obtaining a plurality of outer reverse primers where each primer targets one of the polymorphic loci, and where the outer reverse primers are designed to hybridize to the region of RNA or cDNA derived from the RNA immediately downstream from the inner reverse primer, hybridizing the first primers to the RNA or cDNA derived from the RNA, and amplifying the cDNA using the polymerase chain reaction.

In some exemplary embodiments, the method also includes obtaining a plurality of outer reverse primers where each primer targets one of the polymorphic loci, and where the outer reverse primers are designed to hybridize to the region of RNA or cDNA derived from the RNA immediately downstream from the inner reverse primer, optionally obtaining a plurality of outer forward primers where each primer targets one of the polymorphic loci, and where the outer forward primers are designed to hybridize to the region of RNA or cDNA derived from the RNA upstream from the inner forward primer, hybridizing the first primers to the RNA or cDNA derived from the RNA, and amplifying the RNA or cDNA derived from the RNA using the polymerase chain reaction.

In some exemplary embodiments, preparing the first sample further includes appending universal adapters to the RNA in the first sample and amplifying the RNA in the first sample using the polymerase chain reaction. In some exemplary embodiments, at least a fraction of the amplicons that are amplified are less than 100 bp, less than 90 bp, less than 80 bp, less than 70 bp, less than 65 bp, less than 60 bp, less than 55 bp, less than 50 bp, or less than 45 bp, and where the fraction is 10%, 20%, 30%, 40%, 50%, 60%, 70%, 80%, 90%, or 99%.

In some exemplary embodiments, amplifying the RNA or cDNA derived from the RNA is done in one or a plurality of individual reaction volumes, and where each individual reaction volume contains more than 100 different forward and reverse primer pairs, more than 200 different forward and reverse primer pairs, more than 500 different forward and reverse primer pairs, more than 1,000 different forward and reverse primer pairs, more than 2,000 different forward and reverse primer pairs, more than 5,000 different forward and reverse primer pairs, more than 10,000 different forward and reverse primer pairs, more than 20,000 different forward and reverse primer pairs, more than 50,000 different forward and reverse primer pairs, or more than 100,000 different forward and reverse primer pairs.

In some exemplary embodiments, preparing the sample further comprises dividing the sample into a plurality of portions, and where the RNA or cDNA derived from the RNA in each portion is preferentially enriched at a subset of the plurality of polymorphic loci. In some exemplary embodiments, the inner primers are selected by identifying primer pairs likely to form undesired primer duplexes and removing from the plurality of primers at least one of the pair of primers identified as being likely to form undesired primer duplexes. In some exemplary embodiments, the inner primers contain a region that is designed to hybridize either upstream or downstream of the targeted polymorphic locus, and optionally contain a universal priming sequence designed to allow PCR amplification. In some exemplary embodiments, at least some of the primers additionally contain a random region that differs for each individual primer molecule.

In some exemplary embodiments, at least some of the primers additionally contain a molecular barcode.

In some exemplary embodiments, the method comprises: (a) performing multiplex polymerase chain reaction (PCR) on a nucleic acid sample comprising target loci to simultaneously amplify at least 1,000 distinct target loci using either (i) at least 1,000 different primer pairs, or (ii) at least 1,000 target-specific primers and a universal or tag-specific primer, in a single reaction volume to produce amplified products comprising target amplicons; and (b) sequencing the amplified products. In some exemplary embodiments, the method does not comprise using a microarray.

In some exemplary embodiments, the method comprises (a) performing multiplex polymerase chain reaction (PCR) on the RNA (or cDNA) sample comprising target loci to simultaneously amplify at least 1,000 distinct target loci using either (i) at least 1,000 different primer pairs, or (ii) at least 1,000 target-specific primers and a universal or tag-specific primer, in a single reaction volume to produce amplified products comprising target amplicons; and b) sequencing the amplified products. In some exemplary embodiments, the method does not comprise using a microarray.

In some exemplary embodiments, the method also includes obtaining genotypic data from one or both of the transplant donor and the transplant recipient. In some exemplary embodiments, obtaining genotypic data from one or both of the transplant donor and the transplant recipient includes preparing the RNA from the donor and the recipient where the preparing comprises preferentially enriching the RNA at the plurality of polymorphic loci to give prepared RNA or cDNA, optionally amplifying the prepared cDNA, and measuring the RNA in the prepared sample at the plurality of polymorphic loci.

In some exemplary embodiments, building a joint distribution model for the expected allele count probabilities of the plurality of polymorphic loci on the chromosome is done using the obtained genetic data from the one or both of the transplant donor and the transplant recipient. In some exemplary embodiments, the first sample has been isolated from transplant recipient plasma and where the obtaining genotypic data from the transplant recipient is done by estimating the recipient genotypic data from the RNA measurements made on the prepared sample.

In some exemplary embodiments, preferential enrichment results in average degree of allelic bias between the prepared sample and the first sample of a factor selected from the group consisting of no more than a factor of 2, no more than a factor of 1.5, no more than a factor of 1.2, no more than a factor of 1.1, no more than a factor of 1.05, no more than a factor of 1.02, no more than a factor of 1.01, no more than a factor of 1.005, no more than a factor of 1.002, no more than a factor of 1.001 and no more than a factor of 1.0001. In some exemplary embodiments, the plurality of polymorphic loci are SNPs. In some exemplary embodiments, measuring the RNA in the prepared sample is done by sequencing.

In some exemplary embodiments, a diagnostic box is disclosed for helping to determine transplant status in a transplant recipient where the diagnostic box is capable of executing the preparing and measuring steps of the disclosed methods.

In some exemplary embodiments, the allele counts are probabilistic rather than binary. In some exemplary embodiments, measurements of the RNA in the prepared sample at the plurality of polymorphic loci are also used to determine whether or not the transplant has inherited one or a plurality of linked haplotypes.

In some exemplary embodiments, building a joint distribution model for allele count probabilities is done by using data about the probability of chromosomes crossing over at different locations in a chromosome to model dependence between polymorphic alleles on the chromosome. In some exemplary embodiments, building a joint distribution model for allele counts and the step of determining the relative probability of each hypothesis are done using a method that does not require the use of a reference chromosome.

In some exemplary embodiments, determining the relative probability of each hypothesis makes use of an estimated fraction of donor-derived RNA (dd-RNA) in the prepared sample. In some exemplary embodiments, the DNA measurements from the prepared sample used in calculating allele count probabilities and determining the relative probability of each hypothesis comprise primary genetic data. In some exemplary embodiments, selecting the transplant status corresponding to the hypothesis with the greatest probability is carried out using maximum likelihood estimates or maximum a posteriori estimates.

In some exemplary embodiments, calling the transplant status also includes combining the relative probabilities of each of the status hypotheses determined using the joint distribution model and the allele count probabilities with relative probabilities of each of the status hypotheses that are calculated using statistical techniques taken from a group consisting of a read count analysis, comparing heterozygosity rates, a statistic that is only available when donor genetic information is used, the probability of normalized genotype signals for certain donor/recipient contexts, a statistic that is calculated using an estimated transplant fraction of the first sample or the prepared sample, and combinations thereof.

In some exemplary embodiments, a confidence estimate is calculated for the called transplant status. In some exemplary embodiments, the method also includes taking a clinical action based on the called transplant status.

In some exemplary embodiments, a report displaying a determined transplant status is generated using the method. In some exemplary embodiments, a kit is disclosed for determining a transplant status designed to be used with the methods disclosed herein, the kit including a plurality of inner forward primers and optionally the plurality of inner reverse primers, where each of the primers is designed to hybridize to the region of RNA immediately upstream and/or downstream from one of the target sites, where the region of hybridization is separated from the target site by a small number of bases, where the small number is selected from the group consisting of 1, 2, 3, 4, 5, 6 to 10, 11 to 15, 16 to 20, 21 to 25, 26 to 30, 31 to 60, and combinations thereof.

In some exemplary embodiments, the methods disclosed herein comprise a selection step to select for shorter RNA.

In some exemplary embodiments, the methods disclosed herein comprise a universal application step to enrich for RNA.

In some exemplary embodiments, the determination that the amount of dd-RNA above a cutoff threshold is indicative of acute rejection of the transplant. Machine learning and artificial intelligence may be used to resolve rejection vs non-rejection.

In some exemplary embodiments, the cutoff threshold value is expressed as percentage of dd-RNA (dd-RNA %) in the blood sample.

In some exemplary embodiments, the cutoff threshold value is expressed as copy number of dd-RNA per volume unit of the blood sample.

In some exemplary embodiments, the cutoff threshold value is expressed as copy number of dd-RNA per volume unit of the blood sample multiplied by body mass or blood volume of the transplant recipient.

In some exemplary embodiments, the cutoff threshold value takes into account the body mass or blood volume of the patient.

In some exemplary embodiments, the cutoff threshold value takes into account one or more of the followings: donor genome copies per volume of plasma, cell-free DNA or RNA yield per volume of plasma, donor height, donor weight, donor age, donor gender, donor ethnicity, donor organ mass, donor organ, live vs deceased donor, related vs unrelated donor, recipient height, recipient weight, recipient age, recipient gender, recipient ethnicity, creatinine, eGFR (estimated glomerular filtration rate), cfDNA methylation, DSA (donor-specific antibodies), KDPI (kidney donor profile index), medications (immunosuppression, steroids, blood thinners, etc.), infections (BKV, EBV, CMV, UTI), recipient and/or donor HLA alleles or epitope mismatches, Banff classification of renal allograft pathology, and for-cause vs surveillance or protocol biopsy.

In some exemplary embodiments, the cutoff threshold value is scaled according to the amount of total RNA in the blood sample.

In some exemplary embodiments, the method has a sensitivity of at least 80% in identifying acute rejection (AR) over non-AR when the dd-RNA amount is above the cutoff threshold value scaled according to the amount of total RNA in the sample and a confidence interval of 95%.

In some exemplary embodiments, the method has a specificity of at least 70% in identifying acute rejection (AR) over non-AR when the dd-RNA amount is above the cutoff threshold value scaled according to the amount of total RNA in the blood sample and a confidence interval of 95%.

In some exemplary embodiments, the method has a sensitivity of at least 80% in identifying acute rejection (AR) over non-AR when the dd-RNA amount is above the cutoff threshold value scaled according to the amount of total RNA in the sample and a confidence interval of 95%. In some exemplary embodiments, the method has a sensitivity of at least 85% in identifying acute rejection (AR) over non-AR when the dd-RNA amount is above the cutoff threshold value scaled according to the amount of total RNA in the blood sample and a confidence interval of 95%. In some exemplary embodiments In some exemplary embodiments, the method has a sensitivity of at least 90% in identifying acute rejection (AR) over non-AR when the dd-RNA amount is above the cutoff threshold value scaled according to the amount of total RNA in the blood sample and a confidence interval of 95%. In some exemplary embodiments, the method has a sensitivity of at least 95% in identifying acute rejection (AR) over non-AR when the dd-RNA amount is be above the cutoff threshold value scaled according to the amount of total RNA in the sample and a confidence interval of 95%.

In some exemplary embodiments, the method has a specificity of at least 70% in identifying acute rejection (AR) over non-AR when the RNA amount is above the cutoff threshold value scaled according to the amount of total RNA in the blood sample and a confidence interval of 95%. In some exemplary embodiments, the method has a specificity of at least 75% in identifying acute rejection (AR) over non-AR when the dd-RNA amount is above the cutoff threshold value scaled according to the amount of total RNA in the blood sample and a confidence interval of 95%. In some exemplary embodiments, the method has a specificity of at least 85% in identifying acute rejection (AR) over non-AR when the dd-RNA amount is above the cutoff threshold value scaled according to the amount of total RNA in the blood sample and a confidence interval of 95%. In some exemplary embodiments, the method has a specificity of at least 90% in identifying acute rejection (AR) over non-AR when the dd-cfDNA amount is above the cutoff threshold value scaled according to the amount of total RNA in the blood sample and a confidence interval of 95%. In some exemplary embodiments, the method has a specificity of at least 95% in identifying acute rejection (AR) over non-AR when the dd-RNA amount is above the cutoff threshold value scaled according to the amount of total RNA in the blood sample and a confidence interval of 95%.

### Analysis of Donor-Derived Cell-Free DNA for Monitoring Transplant Rejection

In one aspect, the present invention further comprises: (i) measuring the amount of donor-derived cell-free DNA in a sample obtained from the transplant recipient, extracting cell-free DNA from the sample obtained from the transplant recipient, wherein the extracted cell-free DNA comprises donor-derived cell-free DNA and recipient-derived cell-free DNA; (ii) performing targeted amplification of the extracted DNA at 10-50,000 target loci in a single reaction volume; (iii) sequencing the amplified DNA to obtain sequencing reads and quantifying the amount of donor-derived cell-free DNA based on the sequencing reads, determining transplant rejection based on whether the amount of donor-derived cell-free DNA or a function thereof exceeds a cutoff threshold of cell-free DNA amount that indicates transplant rejection, wherein transplant rejection is determined based on whether both the amount of donor-derived RNA and the amount of donor derived cell-free DNA or function thereof exceeds a cutoff threshold that indicates transplant rejection.

In another aspect, the present invention relates to a method of quantifying the amount of donor-derived cell-free DNA (dd-cfDNA) in a blood sample of a transplant recipient, comprising: extracting DNA from the blood sample of the transplant recipient, wherein the DNA comprises donor-derived cell-free DNA and recipient-derived cell-free DNA; performing targeted amplification at 100-50,000 or 500-50,000 target loci in a single reaction volume using 100-50,000 or 500-50,000 primer pairs, wherein the target loci comprise polymorphic loci and non-polymorphic loci, and wherein each primer pair is designed to amplify a target sequence of no more than 100 bp; and quantifying the amount of donor-derived cell-free DNA in the amplification products.

In another aspect, the present invention relates to a method of quantifying the amount of donor-derived cell-free DNA (dd-cfDNA) in a blood sample of a transplant recipient, comprising: extracting DNA from the blood sample of the transplant recipient, wherein the DNA comprises donor-derived cell-free DNA and recipient-derived cell-free DNA, and wherein the extracting step comprises size selection to enrich for donor-derived cell-free DNA and reduce the amount of recipient-derived cell-free DNA disposed from bursting white-blood cells; performing targeted amplification at 500-50,000 target loci in a single reaction volume using 500-50,000 primer pairs, wherein the target loci comprise polymorphic loci and non-polymorphic loci; and quantifying the amount of donor-derived cell-free DNA in the amplification products.

In another aspect, the present invention relates to a method of detecting donor-derived cell-free DNA (dd-cfDNA) in a blood sample of a transplant recipient, comprising: extracting DNA from the blood sample of the transplant recipient, wherein the DNA comprises donor-derived cell-free DNA and recipient-derived cell-free DNA; performing targeted amplification at 100-50,000 target loci in a single reaction volume using 100-50,000 primer pairs, wherein the target loci comprise polymorphic loci and non-polymorphic loci; sequencing the amplification products by high-throughput sequencing; and quantifying the amount of donor-derived cell-free DNA.

In some exemplary embodiments, the method further comprises performing universal amplification of the extracted DNA. In some exemplary embodiments, the universal amplification preferentially amplifies donor-derived cell-free DNA over recipient-derived cell-free DNA that are disposed from bursting white-blood cells.

In some exemplary embodiments, the transplant recipient is a mammal. In some exemplary embodiments, the transplant recipient is a human.

In some exemplary embodiments, the transplant recipient has received a transplant selected from organ transplant, tissue transplant, cell transplant, and fluid transplant. In some exemplary embodiments, the transplant recipient has received a transplant selected from kidney transplant, liver transplant, pancreas transplant, intestinal transplant, heart transplant, lung transplant, heart/lung transplant, stomach transplant, testis transplant, penis transplant, ovary transplant, uterus transplant, thymus transplant, face transplant, hand transplant, leg transplant, bone transplant, bone marrow transplant, cornea transplant, skin transplant, pancreas islet cell transplant, heart valve transplant, blood vessel transplant, and blood transfusion. In some exemplary embodiments, the transplant recipient has received SPK transplant.

In some exemplary embodiments, the quantifying step comprises determining the percentage of donor-derived cell-free DNA out of the total of donor-derived cell-free DNA and recipient-derived cell-free DNA in the blood sample. In some exemplary embodiments, the quantifying step comprises determining the number of copies of donor-derived cell-free DNA per volume unit of the blood sample.

In some exemplary embodiments, the method further comprises detecting the occurrence or likely occurrence of active rejection of transplantation using the quantified amount of donor-derived cell-free DNA. In some exemplary embodiments, the method is performed without prior knowledge of donor genotypes.

In some exemplary embodiments, each primer pair is designed to amplify a target sequence of about 50-100 bp. In some exemplary embodiments, each primer pair is designed to amplify a target sequence of no more than 75 bp. In some exemplary embodiments, each primer pair is designed to amplify a target sequence of about 60-75 bp. In some exemplary embodiments, each primer pair is designed to amplify a target sequence of about 65 bp.

In some exemplary embodiments, the targeted amplification comprises amplifying at least 1,000 polymorphic loci in a single reaction volume. In some exemplary embodiments, the targeted amplification comprises amplifying at least 2,000 polymorphic loci in a single reaction volume. In some exemplary embodiments, the targeted amplification comprises amplifying at least 5,000 polymorphic loci in a single reaction volume. In some exemplary embodiments, the targeted amplification comprises amplifying at least 10,000 polymorphic loci in a single reaction volume. In some exemplary embodiments, the targeted amplification comprises amplifying 10 to 10,000, 10 to 50,000, 100 to 50,000, or 1000 to 50,000 polymorphic loci in a single reaction volume.

In some exemplary embodiments, method further comprises measuring an amount of one or more alleles at the target loci that are polymorphic loci. In some exemplary embodiments, the polymorphic loci and the non-polymorphic loci are amplified in a single reaction.

In some exemplary embodiments, the quantifying step comprises detecting the amplified target loci using a microarray. In some exemplary embodiments, the quantifying step does not comprise using a microarray.

In some exemplary embodiments, the targeted amplification comprises simultaneously amplifying 500-50,000 target loci in a single reaction volume using (i) at least 500-50,000 different primer pairs, or (ii) at least 500-50,000 target-specific primers and a universal or tag-specific primer 500-50,000 primer pairs.

In a further aspect, the present invention relates to a method of determining the likelihood of transplant rejection within a transplant recipient, the method comprising: extracting DNA from the blood sample of the transplant recipient, wherein the DNA comprises donor-derived cell-free DNA and recipient-derived cell-free DNA; performing universal amplification of the extracted DNA; performing targeted amplification at 500-50,000 target loci in a single reaction volume using 500-50,000 primer pairs, wherein the target loci comprise polymorphic loci and non-polymorphic loci; sequencing the amplification products by high-throughput sequencing; and quantifying the amount of donor-derived cell-free DNA in the blood sample, wherein a greater amount of dd-cfDNA indicates a greater likelihood of transplant rejection.

In a further aspect, the present invention relates to a method of diagnosing a transplant within a transplant recipient as undergoing acute rejection, the method comprising: extracting DNA from the blood sample of the transplant recipient, wherein the DNA comprises donor-derived cell-free DNA and recipient-derived cell-free DNA; performing universal amplification of the extracted DNA; performing targeted amplification at 500-50,000 target loci in a single reaction volume using 500-50,000 primer pairs, wherein the target loci comprise polymorphic loci and non-polymorphic loci; sequencing the amplification products by high-throughput sequencing; and quantifying the amount of donor-derived cell-free DNA in the blood sample, wherein an amount of dd-cfDNA of greater than 1% (or 1.1%, or 1.2%, or 1.3%, or 1.4%, or 1.5%, or 1.6%, or 1.7%, or 1.8%, or 1.9%, or 2.0%) indicates that the transplant is undergoing acute rejection.

In some exemplary embodiments, the transplant rejection is antibody mediated transplant rejection. In some exemplary embodiments, the transplant rejection is T cell mediated transplant rejection.

In some exemplary embodiments, an amount of dd-cfDNA of less than 1% (or 0.9%, or 0.8%, or 0.7%, or 0.6%, or 0.5%) indicates that the transplant is either undergoing borderline rejection, undergoing other injury, or stable.

In a further aspect, the present invention relates to a method of monitoring immunosuppressive therapy in a subject, the method comprising: extracting DNA from the blood sample of the transplant recipient, wherein the DNA comprises donor-derived cell-free DNA and recipient-derived cell-free DNA; performing universal amplification of the extracted DNA; performing targeted amplification at 500-50,000 target loci in a single reaction volume using 500-50,000 primer pairs, wherein the target loci comprise polymorphic loci and non-polymorphic loci; sequencing the amplification products by high-throughput sequencing; and quantifying the amount of donor-derived cell-free DNA in the blood sample, wherein a change in levels of dd-cfDNA over a time interval is indicative of transplant status.

In some exemplary embodiments, the method further comprising adjusting immunosuppressive therapy based on the levels of dd-cfDNA over the time interval.

In some exemplary embodiments, an increase in the levels of dd-cfDNA is indicative of transplant rejection and a need for adjusting immunosuppressive therapy. In some exemplary embodiments, no change or a decrease in the levels of dd-cfDNA indicates transplant tolerance or stability, and a need for adjusting immunosuppressive therapy.

In some exemplary embodiments, an amount of dd-cfDNA of greater than 1% (or 1.1%, or 1.2%, or 1.3%, or 1.4%, or 1.5%, or 1.6%, or 1.7%, or 1.8%, or 1.9%, or 2.0%) indicates that the transplant is undergoing acute rejection. In some exemplary embodiments, the transplant rejection is antibody mediated transplant rejection. In some exemplary embodiments, the transplant rejection is T cell mediated transplant rejection.

In some exemplary embodiments, an amount of dd-cfDNA of less than 1% (or 0.9%, or 0.8%, or 0.7%, or 0.6%, or 0.5%) indicates that the transplant is either undergoing borderline rejection, undergoing other injury, or stable.

In some exemplary embodiments, the method does not comprise genotyping the transplant donor and/or the transplant recipient.

In some exemplary embodiments, the method further comprises measuring an amount of one or more alleles at the target loci that are polymorphic loci.

In some exemplary embodiments, the target loci comprise at least 1,000 polymorphic loci, or at least 2,000 polymorphic loci, or at least 5,000 polymorphic loci, or at least 10,000 polymorphic loci.

In some exemplary embodiments, the target loci that are amplified in amplicons of about 50-100 bp in length, or about 50-90 bp in length, or about 60-80 bp in length, or about 60-75 bp in length, or about 65 bp in length.

In some exemplary embodiments, the transplant recipient is a human. In some exemplary embodiments, the transplant recipient has received a transplant selected from a kidney transplant, liver transplant, pancreas transplant, islet cell transplant, intestinal transplant, heart transplant, lung transplant, bone marrow transplant, heart valve transplant, or a skin transplant. In some exemplary embodiments, the transplant recipient has received SPK transplant.

In some exemplary embodiments, the extracting step comprises size selection to enrich for donor-derived cell-free DNA and reduce the amount of recipient-derived cell-free DNA disposed from bursting white-blood cells.

In some exemplary embodiments, the universal amplification step preferentially amplifies donor-derived cell-free DNA over recipient-derived cell-free DNA that are disposed from bursting white-blood cells.

In some exemplary embodiments, the method comprises longitudinally collecting a plurality of blood samples from the transplant recipient after transplantation, and repeating steps (a) to (e) for each blood sample collected. In some exemplary embodiments, the method comprises collecting and analyzing blood samples from the transplant recipient for a time period of about three months, or about six months, or about twelve months, or about eighteen months, or about twenty-four months, etc. In some exemplary embodiments, the method comprises collecting blood samples from the transplant recipient at an interval of about one week, or about two weeks, or about three weeks, or about one month, or about two months, or about three months, etc.

In some exemplary embodiments, the method has a sensitivity of at least 80%, or at least 85%, or at least 90%, or at least 95%, or at least 98% in identifying acute rejection (AR) over non-AR with a cutoff threshold of 1% dd-cfDNA and a confidence interval of 95%.

In some exemplary embodiments, the method has a specificity of at least 60%, or at least 65%, or at least 70%, or at least 75%, or at least 80%, or at least 85%, or at least 90% in identifying AR over non-AR with a cutoff threshold of 1% dd-cfDNA and a confidence interval of 95%.

In some exemplary embodiments, the method has an area under the curve (AUC) of at least 0.8, or 0.85, or at least 0.9, or at least 0.95 in identifying AR over non-AR with a cutoff threshold of 1% dd-cfDNA and a confidence interval of 95%.

In some exemplary embodiments, the method has a sensitivity of at least 80%, or at least 85%, or at least 90%, or at least 95%, or at least 98% in identifying AR over normal, stable allografts (STA) with a cutoff threshold of 1% dd-cfDNA and a confidence interval of 95%.

In some exemplary embodiments, the method has a specificity of at least 80%, or at least 85%, or at least 90%, or at least 95%, or at least 98% in identifying AR over STA with a cutoff threshold of 1% dd-cfDNA and a confidence interval of 95%.

In some exemplary embodiments, the method has an AUC of at least 0.8, or 0.85, or at least 0.9, or at least 0.95, or at least 0.98, or at least 0.99 in identifying AR over STA with a cutoff threshold of 1% dd-cfDNA and a confidence interval of 95%.

In some exemplary embodiments, the method has a sensitivity as determined by a limit of blank (LoB) of 0.5% or less, and a limit of detection (LoD) of 0.5% or less. In some exemplary embodiments, LoB is 0.23% or less and LoD is 0.29% or less. In some exemplary embodiments, the sensitivity is further determined by a limit of quantitation (LoQ). In some exemplary embodiments, LoQ is 10 times greater than the LoD; LoQ may be 5 times greater than the LoD; LoQ may be 1.5 times greater than the LoD; LoQ may be 1.2 times greater than the LoD; LoQ may be 1.1 times greater than the LoD; or LoQ may be equal to or greater than the LoD. In some exemplary embodiments, LoB is equal to or less than 0.04%, LoD is equal to or less than 0.05%, and/or LoQ is equal to the LoD.

In some exemplary embodiments, the method has an accuracy as determined by evaluating a linearity value obtained from linear regression analysis of measured donor fractions as a function of the corresponding attempted spike levels, wherein the linearity value is a R2 value, wherein the R2 value is from about 0.98 to about 1.0. In some exemplary embodiments, the R2 value is 0.999. In some exemplary embodiments, the method has an accuracy as determined by using linear regression on measured donor fractions as a function of the corresponding attempted spike levels to calculate a slope value and an intercept value, wherein the slope value is from about 0.9 to about 1.2 and the intercept value is from about-0.0001 to about 0.01. In some exemplary embodiments, the slope value is approximately 1, and the intercept value is approximately 0.

In some exemplary embodiments, the method has a precision as determined by calculating a coefficient of variation (CV), wherein the CV is less than about 10.0%. CV is less than about 6%. In some exemplary embodiments, the CV is less than about 4%. In some exemplary embodiments, the CV is less than about 2%. In some exemplary embodiments, the CV is less than about 1%.

In some exemplary embodiments, the AR is antibody-mediated rejection (ABMR). In some exemplary embodiments, the AR is T-cell-mediated rejection (TCMR).

Further disclosed herein are methods for detection of transplant donor-derived cell-free DNA (dd-cfDNA) in a sample from a transplant recipient. In some exemplary embodiments, in the methods disclosed herein, the transplant recipient is a mammal. In some exemplary embodiments, the transplant recipient is a human. In some exemplary embodiments, the transplant recipient has received a transplant selected from a kidney transplant, liver transplant, pancreas transplant, islet cell transplant, intestinal transplant, heart transplant, lung transplant, bone marrow transplant, heart valve transplant, or a skin transplant. In some exemplary embodiments, the transplant recipient has received SPK transplant. In some exemplary embodiments, the method may be performed on transplant recipients the day of or after transplant surgery, up to a year following transplant surgery.

In some exemplary embodiments, disclosed herein is a method of amplifying target loci of donor-derived cell-free DNA (dd-cfDNA) from a blood sample of a transplant recipient, the method comprising: a) extracting DNA from the blood sample of the transplant recipient, wherein the DNA comprises cell-free DNA derived from both the transplanted cells and from the transplant recipient, b) enriching the extracted DNA at target loci, wherein the target loci comprise 50 to 5000 target loci comprising polymorphic loci and non-polymorphic loci; and c) amplifying the target loci.

In some exemplary embodiments, disclosed herein is a method of detecting donor-derived cell-free DNA (dd-cfDNA) in a blood sample from a transplant recipient, the method comprising: a) extracting DNA from the blood sample of the transplant recipient, wherein the DNA comprises cell-free DNA derived from both the transplanted cells and from the transplant recipient, b) enriching the extracted DNA at target loci, wherein the target loci comprise 50 to 5000 target loci comprising polymorphic loci and non-polymorphic loci; c) amplifying the target loci; d) contacting the amplified target loci with probes that specifically hybridize to target loci; and e) detecting binding of the target loci with the probes, thereby detecting dd-cfDNA in the blood sample. In some exemplary embodiments, the probes are labelled with a detectable marker.

In some exemplary embodiments, disclosed herein is a method of determining the likelihood of transplant rejection within a transplant recipient, the method comprising: a) extracting DNA from the blood sample of the transplant recipient, wherein the DNA comprises cell-free DNA derived from both the transplanted cells and from the transplant recipient, b) enriching the extracted DNA at target loci, wherein the target loci comprise 50 to 5000 target loci comprising polymorphic loci and non-polymorphic loci; c) amplifying the target loci; and d) measuring an amount of transplant DNA and an amount of recipient DNA in the recipient blood sample; wherein a greater amount of dd-cfDNA indicates a greater likelihood of transplant rejection.

In some exemplary embodiments, disclosed herein is a method of diagnosing a transplant within a transplant recipient as undergoing acute rejection, the method comprising: a) extracting DNA from the blood sample of the transplant recipient, wherein the DNA comprises cell-free DNA derived from both the transplanted cells and from the transplant recipient, b) enriching the extracted DNA at target loci, wherein the target loci comprise 50 to 5000 target loci comprising polymorphic loci and non-polymorphic loci; c) amplifying the target loci; and d) measuring an amount of transplant DNA and an amount of recipient DNA in the recipient blood sample; wherein an amount of dd-cfDNA of greater than 1% (or 1.1%, or 1.2%, or 1.3%, or 1.4%, or 1.5%, or 1.6%, or 1.7%, or 1.8%, or 1.9%, or 2.0%) indicates that the transplant is undergoing acute rejection.

In some exemplary embodiments, in the methods disclosed herein, the transplant rejection is antibody mediated transplant rejection. In some exemplary embodiments, the transplant rejection is T cell mediated transplant rejection. In some exemplary embodiments, an amount of dd-cfDNA of less than 1% (or 0.9%, or 0.8%, or 0.7%, or 0.6%, or 0.5%) indicates that the transplant is either undergoing borderline rejection, undergoing other injury, or stable.

In some exemplary embodiments, disclosed herein is a method of monitoring immunosuppressive therapy in a subject, the method comprising a) extracting DNA from the blood sample of the transplant recipient, wherein the DNA comprises cell-free DNA derived from both the transplanted cells and from the transplant recipient, b) enriching the extracted DNA at target loci, wherein the target loci comprise 50 to 5000 target loci comprising polymorphic loci and non-polymorphic loci; c) amplifying the target loci; and d) measuring an amount of transplant DNA and an amount of recipient DNA in the recipient blood sample; wherein a change in levels of dd-cfDNA over a time interval is indicative of transplant status. In some exemplary embodiments, the method further comprises adjusting immunosuppressive therapy based on the levels of dd-cfDNA over the time interval. In some exemplary embodiments, an increase in the levels of dd-cfDNA are indicative of transplant rejection and a need for adjusting immunosuppressive therapy. In some exemplary embodiments, a change or a decrease in the levels of dd-cfDNA indicates transplant tolerance or stability, and a need for adjusting immunosuppressive therapy.

In some exemplary embodiments, in the methods disclosed herein, the target loci that are amplified in amplicons of about 50-100 bp in length, or about 60-80 bp in length. In some exemplary embodiments, the amplicons are about 65 bp in length.

In some exemplary embodiments, the methods disclosed herein further comprise measuring an amount of transplant DNA and an amount of recipient DNA in the recipient blood sample.

In some exemplary embodiments, the methods disclosed herein do not comprise genotyping the transplant donor and the transplant recipient.

In at least another aspect, the present invention relates to a method of administrating immunosuppressive therapy in a transplant recipient, comprising: (a) measuring the amount of RNA in a blood, plasma, serum, cerebral spinal fluid (CSF), or urine sample of the transplant recipient; and (b) measuring the amount of donor-derived RNA in a blood, plasma, serum, cerebral spinal fluid (CSF), or urine sample of the transplant recipient; and (c) titrating the dosage of an immunosuppressive therapy according to the amount of RNA or a function thereof and the amount of donor-derived RNA or a function thereof.

In another aspect, the present disclosure relates to a method of administrating immunosuppressive therapy in a transplant recipient, comprising:
(a) identifying a plurality of RNA biomarkers in a sample obtained from the transplant recipient; and
(b) titrating the dosage of an immunosuppressive therapy according to the RNA biomarkers.

In another aspect, the present disclosure relates to a method of administrating immunosuppressive therapy in a transplant recipient, comprising:
(a) identifying a plurality of RNA biomarkers in a sample obtained from the transplant recipient, and detecting an amount of donor-derived RNA; and
(b) titrating the dosage of an immunosuppressive therapy according to the RNA biomarkers and the amount of donor-derived RNA.

In some exemplary embodiments, the method further comprises repeating steps (a)-(b) longitudinally for the same transplant recipient, and determining a longitudinal change in the amount of RNA or a function thereof; and/or a longitudinal change in the amount of donor-derived cell-free DNA or a function thereof.

In some exemplary embodiments, the method further comprises titrating the dosage of the immunosuppressive therapy according to the longitudinal change in the amount of RNA or a function thereof; and/or the longitudinal change in the amount of donor-derived cell-free DNA or a function thereof.

### Analytical Methods

In some exemplary embodiments, the method also includes obtaining genotypic data from one or both of the transplant donor and the transplant recipient. In some exemplary embodiments, obtaining genotypic data from one or both of the transplant donor and the transplant recipient includes preparing the DNA from the donor and the recipient where the preparing comprises preferentially enriching the DNA at the plurality of polymorphic loci to give prepared DNA, optionally amplifying the prepared DNA, and measuring the DNA in the prepared sample at the plurality of polymorphic loci.

In some exemplary embodiments, building a joint distribution model for the expected allele count probabilities of the plurality of polymorphic loci on the chromosome is done using the obtained genetic data from the one or both of the transplant donor and the transplant recipient. In some exemplary embodiments, the first sample has been isolated from transplant recipient plasma and where the obtaining genotypic data from the transplant recipient is done by estimating the recipient genotypic data from the DNA measurements made on the prepared sample.

In some exemplary embodiments, preferential enrichment results in average degree of allelic bias between the prepared sample and the first sample of a factor selected from the group consisting of no more than a factor of 2, no more than a factor of 1.5, no more than a factor of 1.2, no more than a factor of 1.1, no more than a factor of 1.05, no more than a factor of 1.02, no more than a factor of 1.01, no more than a factor of 1.005, no more than a factor of 1.002, no more than a factor of 1.001 and no more than a factor of 1.0001. In some exemplary embodiments, the plurality of polymorphic loci are SNPs. In some exemplary embodiments, measuring the DNA in the prepared sample is done by sequencing.

In some exemplary embodiments, a diagnostic box is disclosed for helping to determine transplant status in a transplant recipient where the diagnostic box is capable of executing the preparing and measuring steps of the disclosed methods.

In some exemplary embodiments, the allele counts are probabilistic rather than binary. In some exemplary embodiments, measurements of the DNA in the prepared sample at the plurality of polymorphic loci are also used to determine whether or not the transplant has inherited one or a plurality of linked haplotypes.

In some exemplary embodiments, building a joint distribution model for allele count probabilities is done by using data about the probability of chromosomes crossing over at different locations in a chromosome to model dependence between polymorphic alleles on the chromosome. In some exemplary embodiments, building a joint distribution model for allele counts and the step of determining the relative probability of each hypothesis are done using a method that does not require the use of a reference chromosome.

In some exemplary embodiments, determining the relative probability of each hypothesis makes use of an estimated fraction of donor-derived RNA and/or the donor-derived cell-free DNA (dd-cfDNA) in the prepared sample. In some exemplary embodiments, the DNA measurements from the prepared sample used in calculating allele count probabilities and determining the relative probability of each hypothesis comprise primary genetic data. In some exemplary embodiments, selecting the transplant status corresponding to the hypothesis with the greatest probability is carried out using maximum likelihood estimates or maximum a posteriori estimates.

In some exemplary embodiments, calling the transplant status also includes combining the relative probabilities of each of the status hypotheses determined using the joint distribution model and the allele count probabilities with relative probabilities of each of the status hypotheses that are calculated using statistical techniques taken from a group consisting of a read count analysis, comparing heterozygosity rates, a statistic that is only available when donor genetic information is used, the probability of normalized genotype signals for certain donor/recipient contexts, a statistic that is calculated using an estimated transplant fraction of the first sample or the prepared sample, and combinations thereof.

In some exemplary embodiments, a confidence estimate is calculated for the called transplant status. In some exemplary embodiments, the method also includes taking a clinical action based on the called transplant status.

In some exemplary embodiments, a report displaying a determined transplant status is generated using the method. In some exemplary embodiments, a kit is disclosed for determining a transplant status designed to be used with the methods disclosed herein, the kit including a plurality of inner forward primers and optionally the plurality of inner reverse primers, where each of the primers is designed to hybridize to the region of DNA immediately upstream and/or downstream from one of the polymorphic sites on the target chromosome, and optionally additional chromosomes, where the region of hybridization is separated from the polymorphic site by a small number of bases, where the small number is selected from the group consisting of 1, 2, 3, 4, 5, 6 to 10, 11 to 15, 16 to 20, 21 to 25, 26 to 30, 31 to 60, and combinations thereof.

In some exemplary embodiments, the cutoff threshold value takes into account one or more of the followings: donor genome copies per volume of plasma, cell-free DNA yield per volume of plasma, donor height, donor weight, donor age, donor gender, donor ethnicity, donor organ mass, donor organ, live vs deceased donor, related vs unrelated donor, recipient height, recipient weight, recipient age, recipient gender, recipient ethnicity, creatinine, eGFR (estimated glomerular filtration rate), cfDNA methylation, DSA (donor-specific antibodies), KDPI (kidney donor profile index), medications (immunosuppression, steroids, blood thinners, etc.), infections (BKV, EBV, CMV, UTI), recipient and/or donor HLA alleles or epitope mismatches, Banff classification of renal allograft pathology, and for-cause vs surveillance or protocol biopsy.

In some exemplary embodiments, the cutoff threshold value is scaled according to the amount of total cfDNA in the blood sample.

In some exemplary embodiments, the method has a sensitivity of at least 80% in identifying acute rejection (AR) over non-AR when the dd-cfDNA amount is above the cutoff threshold value scaled according to the amount of total cfDNA in the blood sample and a confidence interval of 95%.

In some exemplary embodiments, the method has a specificity of at least 70% in identifying acute rejection (AR) over non-AR when the dd-cfDNA amount is above the cutoff threshold value scaled according to the amount of total cfDNA in the blood sample and a confidence interval of 95%.

In some exemplary embodiments, the method has a sensitivity of at least 80% in identifying acute rejection (AR) over non-AR when the dd-cfDNA amount is above the cutoff threshold value scaled according to the amount of total cfDNA in the blood sample and a confidence interval of 95%. In some exemplary embodiments, the method has a sensitivity of at least 85% in identifying acute rejection (AR) over non-AR when the dd-cfDNA amount is above the cutoff threshold value scaled according to the amount of total cfDNA in the blood sample and a confidence interval of 95%. In some exemplary embodiments, the method has a sensitivity of at least 90% in identifying acute rejection (AR) over non-AR when the dd-cfDNA amount is above the cutoff threshold value scaled according to the amount of total cfDNA in the blood sample and a confidence interval of 95%. In some exemplary embodiments, the method has a sensitivity of at least 95% in identifying acute rejection (AR) over non-AR when the dd-cfDNA amount is be above the cutoff threshold value scaled according to the amount of total cfDNA in the blood sample and a confidence interval of 95%.

In some exemplary embodiments, the method has a specificity of at least 70% in identifying acute rejection (AR) over non-AR when the dd-cfDNA amount is above the cutoff threshold value scaled according to the amount of total cfDNA in the blood sample and a confidence interval of 95%. In some exemplary embodiments, the method has a specificity of at least 75% in identifying acute rejection (AR) over non-AR when the dd-cfDNA amount is above the cutoff threshold value scaled according to the amount of total cfDNA in the blood sample and a confidence interval of 95%. In some exemplary embodiments, the method has a specificity of at least 85% in identifying acute rejection (AR) over non-AR when the dd-cfDNA amount is above the cutoff threshold value scaled according to the amount of total cfDNA in the blood sample and a confidence interval of 95%. In some exemplary embodiments, the method has a specificity of at least 90% in identifying acute rejection (AR) over non-AR when the dd-cfDNA amount is above the cutoff threshold value scaled according to the amount of total cfDNA in the blood sample and a confidence interval of 95%. In some exemplary embodiments, the method has a specificity of at least 95% in identifying acute rejection (AR) over non-AR when the dd-cfDNA amount is above the cutoff threshold value scaled according to the amount of total cfDNA in the blood sample and a confidence interval of 95%.

### Multiplex Amplification

In some exemplary embodiments, the method comprises performing a multiplex amplification reaction to amplify a plurality of target loci in one reaction mixture before determining the sequences of the selectively enriched RNA or DNA.

In certain illustrative embodiments, the nucleic acid sequence data is generated by performing high throughput RNA sequencing of a plurality of copies of a series of amplicons generated using a multiplex amplification reaction, wherein each amplicon of the series of amplicons spans at least one polymorphic locus of the set of polymorphic loci and wherein each of the polymeric loci of the set is amplified. In certain illustrative embodiments, the nucleic acid sequence data is generated by performing high throughput DNA sequencing of a plurality of copies of a series of amplicons generated using a multiplex amplification reaction, wherein each amplicon of the series of amplicons spans at least one polymorphic locus of the set of polymorphic loci and wherein each of the polymeric loci of the set is amplified. For example, in these embodiments a multiplex PCR to amplify amplicons across at least 100; 200; 500; 1,000; 2,000; 5,000; 10,000; 20,000; 50,000; or 100,000 polymorphic loci (e.g., SNP loci) may be performed. This multiplex reaction can be set up as a single reaction or as pools of different subset multiplex reactions. The multiplex reaction methods provided herein, such as the massive multiplex PCR disclosed herein provide an exemplary process for carrying out the amplification reaction to help attain improved multiplexing and therefore, sensitivity levels.

In some exemplary embodiments, amplification is performed using direct multiplexed PCR, sequential PCR, nested PCR, doubly nested PCR, one-anda-half sided nested PCR, fully nested PCR, one sided fully nested PCR, one-sided nested PCR, hemi-nested PCR, hemi-nested PCR, triply hemi-nested PCR, semi-nested PCR, one sided semi-nested PCR, reverse semi-nested PCR method, or one-sided PCR, which are described in U.S. application Ser. No. 13/683,604, filed Nov. 21, 2012, U.S. Publication No. 2013/0123120, U.S. application Ser. No. 13/300,235, filed Nov. 18, 2011, U.S. Publication No 2012/0270212, and U.S. Ser. No. 61/994,791, filed May 16, 2014, all of which are hereby incorporated by reference in their entirety.

In some exemplary embodiments, multiplex PCR is used. In some exemplary embodiments, the method of amplifying target loci in a nucleic acid sample involves (i) contacting the nucleic acid sample with a library of primers that simultaneously hybridize to at least 100; 200; 500; 1,000; 2,000; 5,000; 10,000; 20,000; 50,000; or 100,000 different target loci to produce a single reaction mixture; and (ii) subjecting the reaction mixture to primer extension reaction conditions (such as PCR conditions) to produce amplified products that include target amplicons. In some exemplary embodiments, at least 50, 60, 70, 80, 90, 95, 96, 97, 98, 99, or 99.5% of the targeted loci are amplified. In various embodiments, less than 60, 50, 40, 30, 20, 10, 5, 4, 3, 2, 1, 0.5, 0.25, 0.1, or 0.05% of the amplified products are primer dimers. In some exemplary embodiments, the primers are in solution (such as being dissolved in the liquid phase rather than in a solid phase). In some exemplary embodiments, the primers are in solution and are not immobilized on a solid support. In some exemplary embodiments, the primers are not part of a microarray.

In certain embodiments, the multiplex amplification reaction is performed under limiting primer conditions for at least ½ of the reactions. In some exemplary embodiments, limiting primer concentrations are used in 1/10, ⅕, ¼, ⅓, ½, or all of the reactions of the multiplex reaction. Provided herein are factors to consider in achieving limiting primer conditions in an amplification reaction such as PCR.

In certain embodiments, the multiplex amplification reaction can include, for example, between 2,500 and 50,000 multiplex reactions. In certain embodiments, the following ranges of multiplex reactions are performed: between 100, 200, 250, 500, 1000, 2500, 5000, 10,000, 20,000, 25000, 50000 on the low end of the range and between 200, 250, 500, 1000, 2500, 5000, 10,000, 20,000, 25000, 50000, and 100,000 on the high end of the range.

In an embodiment, a multiplex PCR assay is designed to amplify potentially heterozygous SNP or other polymorphic or non-polymorphic loci on one or more chromosomes and these assays are used in a single reaction to amplify DNA. The number of PCR assays may be between 50 and 200 PCR assays, between 200 and 1,000 PCR assays, between 1,000 and 5,000 PCR assays, or between 5,000 and 20,000 PCR assays (50 to 200-plex, 200 to 1,000-plex, 1,000 to 5,000-plex, 5,000 to 20,000-plex, more than 20,000-plex respectively). In an embodiment, a multiplex pool of at least 10,000 PCR assays (10,000-plex) are designed to amplify potentially heterozygous SNP loci a single reaction to amplify cfDNA obtained from a blood, plasma, serum, solid tissue, or urine sample. The SNP frequencies of each locus may be determined by clonal or some other method of sequencing of the amplicons. In another embodiment the original cfDNA samples is split into two samples and parallel 5,000-plex assays are performed. In another embodiment the original cfDNA samples is split into n samples and parallel (˜10,000/n)-plex assays are performed where n is between 2 and 12, or between 12 and 24, or between 24 and 48, or between 48 and 96.

In an embodiment, a method disclosed herein uses highly efficient highly multiplexed targeted PCR to amplify DNA followed by high throughput sequencing to determine the allele frequencies at each target locus. One technique that allows highly multiplexed targeted PCR to perform in a highly efficient manner involves designing primers that are unlikely to hybridize with one another. The PCR probes, typically referred to as primers, are selected by creating a thermodynamic model of potentially adverse interactions between at least 100, at least 200, at least 500, at least 1,000, at least 2,000, at least 5,000, at least 10,000, at least 20,000, or at least 50,000 potential primer pairs, or unintended interactions between primers and sample DNA, and then using the model to eliminate designs that are incompatible with other the designs in the pool. Another technique that allows highly multiplexed targeted PCR to perform in a highly efficient manner is using a partial or full nesting approach to the targeted PCR. Using one or a combination of these approaches allows multiplexing of at least 100, at least 200, at least 500, at least 1,000, at least 2,000, at least 5,000, at least 10,000, at least 20,000, or at least 50,000 primers in a single pool with the resulting amplified DNA comprising a majority of DNA molecules that, when sequenced, will map to targeted loci. Using one or a combination of these approaches allows multiplexing of a large number of primers in a single pool with the resulting amplified DNA comprising greater than 50%, greater than 80%, greater than 90%, greater than 95%, greater than 98%, or greater than 99% DNA molecules that map to targeted loci.

Bioinformatics methods are used to analyze the genetic data obtained from multiplex PCR. The bioinformatics methods useful and relevant to the methods disclosed herein can be found in U.S. Patent Publication No. 2018/0025109, incorporated by reference herein.

### High-Throughput Sequencing

In some exemplary embodiments, the sequences of the amplicons are determined by performing high-throughput sequencing.

The genetic data of the transplant recipient and/or of the transplant donor can be transformed from a molecular state to an electronic state by measuring the appropriate genetic material using tools and or techniques taken from a group including, but not limited to: genotyping microarrays, and high throughput sequencing. Some high throughput sequencing methods include Sanger DNA sequencing, pyrosequencing, the ILLUMINA SOLEXA platform, ILLUMINA'S GENOME ANALYZER, or APPLIED BIOSYSTEM's 454 sequencing platform, HELICOS's TRUE SINGLE MOLECULE SEQUENCING platform, HALCYON MOLECULAR's electron microscope sequencing method, or any other sequencing method. In some exemplary embodiments, the high throughput sequencing is performed on Illumina NextSeq®, followed by demultiplexing and mapping to the human reference genome. All of these methods physically transform the genetic data stored in a sample of DNA into a set of genetic data that is typically stored in a memory device en route to being processed.

In some exemplary embodiments, the sequences of the selectively enriched DNA are determined by performing microarray analysis. In an embodiment, the microarray may be an ILLUMINA SNP microarray, or an AFFYMETRIX SNP microarray.

In some exemplary embodiments, the sequences of the selectively enriched DNA are determined by performing quantitative PCR (qPCR) or digital droplet PCR (ddPCR) analysis. qPCR measures the intensity of fluorescence at specific times (generally after every amplification cycle) to determine the relative amount of target molecule (DNA). ddPCR measures the actual number of molecules (target DNA) as each molecule is in one droplet, thus making it a discrete “digital” measurement. It provides absolute quantification because ddPCR measures the positive fraction of samples, which is the number of droplets that are fluorescing due to proper amplification. This positive fraction accurately indicates the initial amount of template nucleic acid.

### Tracer DNA and Use Thereof

Tracer DNA for estimating the amount of total cfDNA in a sample is described in U.S. Prov. Appl. No. 63/031,879 filed May 29, 2020 and titled “Improved Methods for Detection of Donor Derived Cell-Free DNA”, which is incorporated herein by reference in its entirety. In some exemplary embodiments, the Tracer DNA comprises synthetic double-stranded DNA molecules. In some exemplary embodiments, the Tracer DNA comprises DNA molecules of non-human origin.

In some exemplary embodiments, the Tracer DNA comprises DNA molecules having a length of about 50-500 bp, or about 75-300 bp, or about 100-250 bp, or about 125-200 bp, or about 125 bp, or about 160 bp, or about 200 bp, or about 500-1,000 bp.

In some exemplary embodiments, the Tracer DNA comprises DNA molecules having the same or substantially the same length, such as a DNA molecule having a length of about 125 bp, or about 160 bp, or about 200 bp. In some exemplary embodiments, the Tracer DNA comprises DNA molecules having different lengths, such as a first DNA molecule having a length of about 125 bp, a second DNA molecule having a length of about 160 bp, and a third DNA molecule having a length of about 200 bp. In some exemplary embodiments, the DNA molecules having different lengths are used to determine size distribution of the cell-free DNA in the sample

In some exemplary embodiments, the Tracer DNA comprises a target sequence, wherein the target sequence comprises a barcode positioned between a pair of primer binding sites capable of binding to a pair of primers. In some exemplary embodiments, at least part of the Tracer DNA is designed based on an endogenous human SNP locus, by replacing an endogenous sequence containing the SNP locus with the barcode. During the mmPCR target enrichment step, the primer pair targeting the SNP locus can also amplify the portion of Tracer DNA containing the barcode.

In some exemplary embodiments, the barcode is an arbitrary barcode. In some exemplary embodiments, the barcode comprises reverse complement of a corresponding endogenous genome sequence capable of being amplified by the same primer pair.

In some exemplary embodiments, the target sequence within the Tracer DNA is flanked on one or both sides by endogenous genome sequences. In some exemplary embodiments, the target sequence within the Tracer DNA is flanked on one or both sides by non-endogenous sequences.

In some exemplary embodiments, the Tracer DNA comprises a plurality of target sequences. In some exemplary embodiments, the Tracer DNA comprises a first target sequence comprising a first barcode positioned between a first pair of primer binding sites capable of binding to a first pair of primers, and a second barcode positioned between a second pair of primer binding sites capable of binding to a second pair of primers. In some exemplary embodiments, the first and/or second target sequence is designed based on one or more endogenous human SNP loci, by replacing an endogenous sequence containing a SNP locus with a barcode. In some exemplary embodiments, the first and/or second barcode is an arbitrary barcode. In some exemplary embodiments, the first and/or second barcode comprises reverse complement of a corresponding endogenous genome sequence capable of being amplified by the first or second primer pair. In some exemplary embodiments, the first and/or second target sequence within the Tracer DNA is flanked on one or both sides by endogenous genome sequences. In some exemplary embodiments, the first and/or second target sequence within the Tracer DNA is flanked on one or both sides by non-endogenous sequences.

In some exemplary embodiments, the Tracer DNA comprises DNA molecules having the same or substantially the same sequence. In some exemplary embodiments, the Tracer DNA comprises DNA molecules having different sequences.

In some exemplary embodiments, the Tracer DNA comprises a first DNA comprising a first target sequence and a second DNA comprising a second target sequence. In some exemplary embodiments, the first target sequence and second target sequence have different barcodes positioned between the same primer binding sites. In some exemplary embodiments, the first target sequence and second target sequence have different barcodes positioned between the same primer binding sites, wherein the different barcodes have the same or substantially the same lengths. In some exemplary embodiments, the first target sequence and second target sequence have different barcodes positioned between the same primer binding sites, wherein the different barcodes have different lengths. In some exemplary embodiments, the first target sequence and second target sequence are designed based on different endogenous human SNP loci, and hence comprise different primer binding sites. In some exemplary embodiments, the amount of first DNA and the amount of the second DNA are the same or substantially the same in the Tracer DNA. In some exemplary embodiments, the amount of first DNA and the amount of the second DNA are different in the Tracer DNA.

In certain embodiments, Tracer DNA can be used to improve accuracy and precision of the method described herein, help quantify over a wider input range, assess efficiency of different steps at different size ranges, and/or calculate fragment size-distribution of input material.

Some embodiments of the present invention relate to a method of quantifying the amount of total cell-free DNA in a biological sample, comprising: a) isolating cell-free DNA from the biological sample, wherein a first Tracer DNA is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of total cell-free DNA using sequencing reads derived from the first Tracer DNA.

In some exemplary embodiments, the method comprises adding the first Tracer DNA to a whole blood sample before plasma extraction. In some exemplary embodiments, the method comprises adding the first Tracer DNA to a plasma sample after plasma extraction and before isolation of the cell-free DNA. In some exemplary embodiments, the method comprises adding the first Tracer DNA to a composition comprising the isolated cell-free DNA. In some exemplary embodiments, the method comprises ligating adaptors to the isolated cell-free DNA to obtain a composition comprising adaptor-ligated DNA, and adding the first Tracer DNA to the composition comprising adaptor-ligated DNA.

In some exemplary embodiments, the method further comprises adding a second Tracer DNA before the targeted amplification. In some exemplary embodiments, the method further comprises adding a second Tracer DNA after the targeted amplification.

In some exemplary embodiments, the amount of total cfDNA in the sample is estimated using the NOR of the Tracer DNA (identifiable by the barcode), the NOR of sample DNA, and the known amount of the Tracer DNA added to the plasma sample. In some exemplary embodiments, the ratio between the NOR of the Tracer DNA and the NOR of sample DNA is used to quantify the amount of total cell-free DNA. In some exemplary embodiments, the ratio between the NOR of the barcode and the NOR of the corresponding endogenous genome sequence is used to quantify the amount of total cell-free DNA. In some exemplary embodiments, this information along with the plasma volume can also be used to calculate the amount of cfDNA per volume of plasma. In some exemplary embodiments, these can be multiplied by the percentage of donor DNA to calculate the total donor cfDNA and the donor cfDNA per volume of plasma.

Accordingly, in another aspect, the present invention relates to a method of quantifying the amount of total cell-free DNA in a biological sample, comprising: a) isolating cell-free DNA from the biological sample, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of total cell-free DNA using sequencing reads derived from the first Tracer DNA composition.

In further aspect, the present invention relates to a method of quantifying the amount of donor-derived cell-free DNA in a biological sample of a transplant recipient, comprising: a) isolating cell-free DNA from the biological sample of the transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; and d) quantifying the amount of donor-derived cell-free DNA and the amount of total cell-free DNA, wherein the amount of total cell-free DNA is quantified using sequencing reads derived from the first Tracer DNA composition.

In a further aspect, the present invention relates to a method of determining the occurrence or likely occurrence of transplant rejection, comprising: a) isolating cell-free DNA from a biological sample of a transplant recipient, wherein the isolated cell-free DNA comprises donor-derived cell-free DNA and recipient-derived cell-free DNA, wherein a first Tracer DNA composition is added before or after isolation of the cell-free DNA; b) performing targeted amplification at 100 or more different target loci in a single reaction volume using 100 or more different primer pairs; c) sequencing the amplification products by high-throughput sequencing to generate sequencing reads; d) quantifying the amount of donor-derived cell-free DNA and the amount of total cell-free DNA, wherein the amount of total cell-free DNA is quantified using sequencing reads derived from the first Tracer DNA composition, and determining the occurrence or likely occurrence of transplant rejection using the amount of donor-derived cell-free DNA by comparing the amount of donor-derived cell-free DNA to a threshold value, wherein the threshold value is determined according to the amount of total cell-free DNA.

In some exemplary embodiments, the threshold value is a function of the number of sequencing reads of the donor-derived cell-free DNA.

In some exemplary embodiments, the method further comprises flagging the sample if the amount of total cell-free DNA falls outside a pre-determined range. In some exemplary embodiments, the method further comprises flagging the sample if the amount of total cell-free DNA is above a pre-determined value. In some exemplary embodiments, the method further comprises flagging the sample if the amount of total cell-free DNA is below a pre-determined value.

In some exemplary embodiments, the method comprises adding the first Tracer DNA composition to a whole blood sample before plasma extraction. In some exemplary embodiments, the method comprises adding the first Tracer DNA composition to a plasma sample after plasma extraction and before isolation of the cell-free DNA. In some exemplary embodiments, the method comprises adding the first Tracer DNA composition to a composition comprising the isolated cell-free DNA. In some exemplary embodiments, the method comprises ligating adaptors to the isolated cell-free DNA to obtain a composition comprising adaptor-ligated DNA, and adding the first Tracer DNA composition to the composition comprising adaptor-ligated DNA.

In some exemplary embodiments, the method further comprises adding a second Tracer DNA composition before the targeted amplification. In some exemplary embodiments, the method further comprises adding a second Tracer DNA composition after the targeted amplification.

In some exemplary embodiments, the first and/or second Tracer DNA composition comprises a plurality of DNA molecules having different sequences.

In some exemplary embodiments, the first and/or second Tracer DNA composition comprises a plurality of DNA molecules having at different concentrations.

In some exemplary embodiments, the first and/or second Tracer DNA composition comprises a plurality of DNA molecules having different lengths. In some exemplary embodiments, the plurality of DNA molecules having different lengths are used to determine size distribution of the cell-free DNA in the sample.

In some exemplary embodiments, the first and/or second Tracer DNA composition comprises a plurality of DNA molecules of non-human origin.

In some exemplary embodiments, the first and/or second Tracer DNA composition each comprises a target sequence, wherein the target sequence comprises a barcode positioned between a pair of primer binding sites capable of binding to one of the primer pairs. In some exemplary embodiments, the barcode comprises reverse complement of a corresponding endogenous genome sequence capable of being amplified by the same primer pair.

In some exemplary embodiments, the ratio between the number of reads of the Tracer DNA and the number of reads of sample DNA is used to quantify the amount of total cell-free DNA. In some exemplary embodiments, the ratio between the number of reads of the barcode and the number of reads of the corresponding endogenous genome sequence is used to quantify the amount of total cell-free DNA.

In some exemplary embodiments, the target sequence is flanked on one or both sides by endogenous genome sequences. In some exemplary embodiments, the target sequence is flanked on one or both sides by non-endogenous sequences.

In some exemplary embodiments, the first and/or second Tracer DNA composition comprises synthetic double-stranded DNA molecules. In some exemplary embodiments, the first and/or second Tracer DNA composition comprises DNA molecules having a length of 50-500 bp. In some exemplary embodiments, the first and/or second Tracer DNA composition comprises DNA molecules having a length of 75-300 bp. In some exemplary embodiments, the first and/or second Tracer DNA composition comprises DNA molecules having a length of 100-250 bp. In some exemplary embodiments, the first and/or second Tracer DNA composition comprises DNA molecules having a length of 125-200 bp. In some exemplary embodiments, the first and/or second Tracer DNA composition comprises DNA molecules having a length of about 200 bp. In some exemplary embodiments, the first and/or second Tracer DNA composition comprises DNA molecules having a length of about 160 bp. In some exemplary embodiments, the first and/or second Tracer DNA composition comprises DNA molecules having a length of about 125 bp. In some exemplary embodiments, the first and/or second Tracer DNA composition comprises DNA molecules having a length of 500-1,000 bp.

In some exemplary embodiments, the targeted amplification comprises amplifying at least 100 polymorphic or SNP loci in a single reaction volume. In some exemplary embodiments, the targeted amplification comprises amplifying at least 200 polymorphic or SNP loci in a single reaction volume. In some exemplary embodiments, the targeted amplification comprises amplifying at least 500 polymorphic or SNP loci in a single reaction volume. In some exemplary embodiments, the targeted amplification comprises amplifying at least 1,000 polymorphic or SNP loci in a single reaction volume. In some exemplary embodiments, the targeted amplification comprises amplifying at least 2,000 polymorphic or SNP loci in a single reaction volume. In some exemplary embodiments, the targeted amplification comprises amplifying at least 5,000 polymorphic or SNP loci in a single reaction volume. In some exemplary embodiments, the targeted amplification comprises amplifying at least 10,000 polymorphic or SNP loci in a single reaction volume.

In some exemplary embodiments, each primer pair is designed to amplify a target sequence of about 35 to 200 bp. In some exemplary embodiments, each primer pair is designed to amplify a target sequence of about 50 to 100 bp. In some exemplary embodiments, each primer pair is designed to amplify a target sequence of about 60 to 75 bp. In some exemplary embodiments, each primer pair is designed to amplify a target sequence of about 65 bp.

In some exemplary embodiments, the transplant recipient is a human subject. In some exemplary embodiments, the transplant recipient received an allograft. In some exemplary embodiments, the transplant recipient received a xenograft.

In some exemplary embodiments, the transplant is a human transplant. In some exemplary embodiments, the transplant is a pig transplant. In some exemplary embodiments, the transplant is from a non-human animal.

In some exemplary embodiments, the transplant is an organ transplant, tissue transplant, or cell transplant. In some exemplary embodiments, the transplant is a kidney transplant, liver transplant, pancreas transplant, intestinal transplant, heart transplant, lung transplant, heart/lung transplant, stomach transplant, testis transplant, penis transplant, ovary transplant, uterus transplant, thymus transplant, face transplant, hand transplant, leg transplant, bone transplant, bone marrow transplant, cornea transplant, skin transplant, pancreas islet cell transplant, heart valve transplant, blood vessel transplant, or blood transfusion.

In some exemplary embodiments, the method further comprises determine the transplant rejection as antibody mediated transplant rejection, T-cell mediated transplant rejection, graft injury, viral infection, bacterial infection, or borderline rejection.

### Use for Cancer Screening, Detection, and Monitoring

In some exemplary embodiments, the method further comprises determining the likelihood of one or more cancers. Cancer screening, detection, and monitoring are disclosed in PCT Patent Publication Nos. WO2015/164432, WO2017/181202, WO2018/083467, and WO2019/200228, each of which is incorporated herein by reference in its entirety. In other embodiments, the invention relates to screening a patient to determine their predicted responsiveness, or resistance, to one or more cancer treatments. This determination can be made by determining the existence of wild-type vs. mutated forms of a target gene, or in some cases the increased or over-expression of a target gene. Examples of such target screens include KRAS, NRAS, EGFR, ALK, KIT, and others. For example, a variety of KRAS mutations are appropriate for screening in accordance with the invention including, but not limited to, G12C, G12D, G12V, G13C, G13D, A18D, Q61H, K117N. In addition, PCT Patent Publication Nos. WO2015/164432, WO2017/181202, WO2018/083467, and WO2019/200228, which are incorporated herein by reference in their entirety.

In some exemplary embodiments, the method is performed without prior knowledge of donor genotypes. In some exemplary embodiments, the method is performed without prior knowledge of recipient genotypes. In some exemplary embodiments, the method is performed without prior knowledge of donor and/or recipient genotypes. In some exemplary embodiments, no genotyping of either the donor or the recipient is required prior to performing the method.

In some exemplary embodiments, the biological sample is a blood sample. In some exemplary embodiments, the biological sample is a plasma sample. In some exemplary embodiments, the biological sample is a serum sample. In some exemplary embodiments, the biological sample is a urine sample. In some exemplary embodiments, the biological sample is a sample of lymphatic fluid. In some exemplary embodiments, the sample is a solid tissue sample.

In some exemplary embodiments, the disease or the disease status comprises a cancer, recurrence or metastasis of the cancer, an immune disease or disorder, pre-eclampsia, or congenital heart disease (CHD). In some exemplary embodiments, the cancer is breast cancer, lung cancer, liver cancer, skin cancer, prostate cancer, bladder cancer, or colorectal cancer.

### WORKING EXAMPLES

### Example 1

This example is illustrative only, and a skilled artisan will appreciate that the invention disclosed herein can be practiced in a variety of other ways.

### Blood Samples

Male and female adult or young-adult patients receives a donor organ from related or unrelated living donors, or unrelated deceased donors. Time points of patient blood draw following transplantation surgery are either at the time of an allograft biopsy or at various pre-specified time intervals based on lab protocols. Typically, samples are biopsy-matched and blood are drawn at the time of clinical dysfunction and biopsy or at the time of protocol biopsy (at which time most patients do not have clinical dysfunction). In addition, some patients had serial post transplantation blood drawn. The selection of study samples was based on (a) adequate plasma being available, and (b) if the sample was associated with biopsy information. Among the full 300 sample cohort, 72.3% were drawn on the day of biopsy.

Nucleic acid Measurement in Blood Samples

Nucleic acids such as RNA or DNA, and in particular cell-free DNA, mRNA, and microRNA is extracted from plasma samples using the QIAamp™ Circulating Nucleic Acid Kit (Qiagen) and the LabChip™ NGS 5k kit (Perkin Elmer, Waltham, MA, USA) is used for quantification. Library preparation is performed using the Natera Library Prep kit as described in Abbosh et al, Nature 545:446-451 (2017), with a modification of 18 cycles of library amplification to plateau the libraries. Purified libraries are quantified using LabChip™ NGS 5k as described in Abbosh et al, Nature 545:446-451 (2017). Target enrichment is accomplished using massively multiplexed-PCR (mmPCR) using a modified version of a described in Zimmermann et al., Prenat. Diagn. 32:1233-1241 (2012), with 13,392 single nucleotide polymorphisms (SNPs) targeted. Amplicons are then sequenced on an Illumina HiSeq 2500 Rapid Run®, 50 cycles single end, with 10-11 million reads per sample.

Statistical Analyses of Nucleic Acids, Dd-cfDNA and eGFR

In each sample, donor derived RNA and/or dd-cfDNA was measured and correlated with rejection status, and results are compared with eGFR. Where applicable, all statistical tests are two sided. Significance is set at p<0.05. Because the distribution of dd-cfDNA in patients is severely skewed among the groups, data are analyzed using a Kruskal-Wallis rank sum test followed by Dunn multiple comparison tests with Holm correction. eGFR (serum creatinine in mg/dL) is calculated as described previously for adult and pediatric patients. Briefly, eGFR=186×Serum Creatinine-1.154×Age-0.203×(1.210 if Black)×(0.742 if Female).

### Example 2

This example is illustrative only, and a skilled artisan will appreciate that the invention disclosed herein can be practiced in a variety of other ways. The workflow described in Example 1 is modified by utilizing Cas9/Cas12a to deplete contaminating or overabundant species in whole blood or hemolysis tainted blood, serum or plasma samples, thereby increasing a detection rate of the target loci. The contaminating or overabundant species may comprise hemoglobin mRNA and miR-451, miR-144, and miR-486, thereby increasing the fraction of desired reads that map to a target loci of interest per sample and the sample throughput per sequencing run.

Untargeted miRNA analysis may be performed using the RealSeq® BioSciences technology. Untargeted miRNA analysis may involve ligating the miRNA to an adaptor.

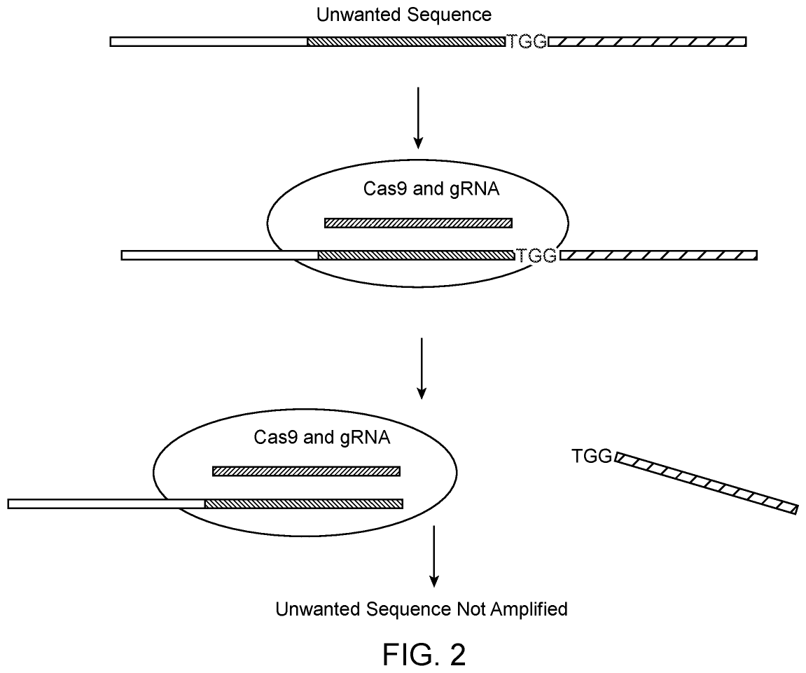

The adaptor may provide a NGG location adjacent to the miRNA to allow for Cas9 cutting. When an unwanted sequence is ligated to the adaptor, a Cas9-gRNA complex complementary to this unwanted sequence will be introduced (see FIG. 2). This will result in removal of the adaptor, preventing PCR amplification. This step should be performed on double-stranded reverse transcribed DNA.

In some exemplary embodiments, a pilot sequencing run can be used to inform which targets need to be removed from future sequencing runs. This would allow assay developers to focus on only miRNAs of interest by designing gRNAs targeting the most common uninteresting or contaminating small RNA fragments actually observed in the specific sample type used in the assay. This could include highly abundant miRNAs or small fragments of ribosomal or messenger RNAs that might cause background noise.

In some exemplary embodiments, Cas9 can be removed by using thermolabile Proteinase K (NEB P8111S) because Cas9 complexes are relatively long-lived and may interfere with downstream applications.

While this approach could also be used in targeted miRNA applications, it is strongly recommended to improve/remove bad primers prior to the assay rather than try to remove them after amplification using CRISPR-Cas.

In some exemplary embodiments, the CRISPER-Cas mediated removal of contaminating species may also be used in mRNA applications. In some exemplary embodiments, CRISPR-Cas approaches are used to remove contaminating mRNA species with a length less than 20 nucleotides. When the length of the contaminating mRNA species is less than 20 nucleotides, a similar approach to that proposed for miRNA could be used to design assays to specifically target the observed sequences. This could be especially useful if, for example, barcoding primer dimers are consuming a large portion of the sequencing reads.

In some exemplary embodiments, CRISPR-Cas approaches are used to remove contaminating mRNA species with a length larger than 20 nucleotides. In some exemplary embodiments, CRISPR-Cas approaches are used in combination with a “tag and capture” method to remove contaminating mRNA species with a length larger than 20 nucleotides.

In some exemplary embodiments, the CRISPR-Cas method to remove contaminating species is used on cDNA derived from miRNA or mRNA prior to amplification. In some exemplary embodiments, the CRISPR-Cas method to remove contaminating species is used on cDNA derived from miRNA or mRNA, and wherein the cDNA is amplified for 1 to 5 cycles, 1 to 10 cycles, or 1-15, cycles. In some exemplary embodiments, the cDNA is amplified for no more than 2, 3, 4, 5, 6, 7, 8, 9, or 10 cycles prior to CRISPR-Cas mediated removal of contaminating species.

### Example 3

This example showed identification of possible RNA (e.g. miRNA) targets for determination of transplant organ health.

Several approaches may be used for identifying RNA targets broadly categorized into non-randomized and randomized methods.

Non-randomized methods include Frequency ratio methods that rank RNAs (e.g. miRNAs) by the ratio of the frequency of mention in papers for the disease of interest with respect to the frequency of mention for other diseases. Non-randomized methods for identifying RNA targets may also include Bayes methods that rank miRNAs by the empirical conditional probabilities defined below:

Interpretation: if one selects a particular miRNA from the dataset, what is the probability of its mention coming from the papers for the diseases of interest? (Bayes rule, i.e. P (Disease of InterestlmiRNA)=P (miRNA|Disease of Interest)×P (Disease of Interest)/P (miRNA)):
2.1 P (miRNA) is estimated as the frequency of mention in papers for each miRNA across diseases;
2.2 P (Disease of Interest) is estimated as the frequency of the diseases of interest mention w.r.t. the total number of mention for all the diseases;
2.3 P (miRNA|Disease of Interest) is estimated as the frequency of mention of each miRNA for the diseases of interest.

Randomized (or simulation) approaches may including repeating (e.g. 1000 times):
1. Frequency Ratio Simulations: rank miRNAs by the ratio of the frequency of mention in papers for the disease of interest with respect to the frequency of mention for randomly selected other diseases set of equal size (bootstrapped samples for the other group, average frequency is recorded in 1000 runs);
2. Bayes Simulations: produce averaged probabilities across the 1000 runs.

Probabilities are higher than in 1 as the number of the diseases of interest equals the number of other diseases by how the simulation is defined (P (Disease of Interest) in 2.1).

The miRNA targets of interest of transplant organ health was identified in one illustrative embodiment by text mining to identify the occurrence of microRNA in organ health tissues of interest compared to non-organ health tissues and diseases. The database included 19000 microRNA papers predigested into 850 disease states. Specificity statistics was used to identify the occurrence of microRNA in organ health tissues of interest compared to non-organ health tissues and diseases. In particular, frequency ratio and Bayesian probabilities of disease miRNA/non-disease miRNA were calculated to identify the occurrence of microRNA in organ health tissues of interest compared to non-organ heath tissues and diseases. In this test case, higher confidence miRNA were selected by plotting the Frequency ratios and Bayesian probabilities of the occurrence of a miRNA in a specific disease state compared to a non-specific disease state. Data was plotted and varying specificity thresholds were determined from plots of frequency ratio, frequency ratio simulated, Bayes, and Bayes simulated.

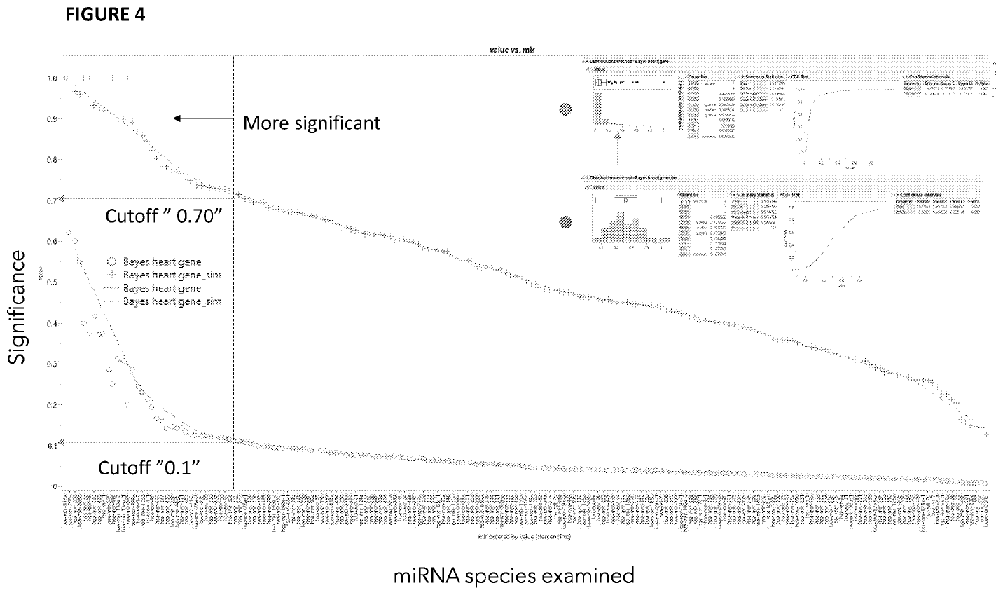

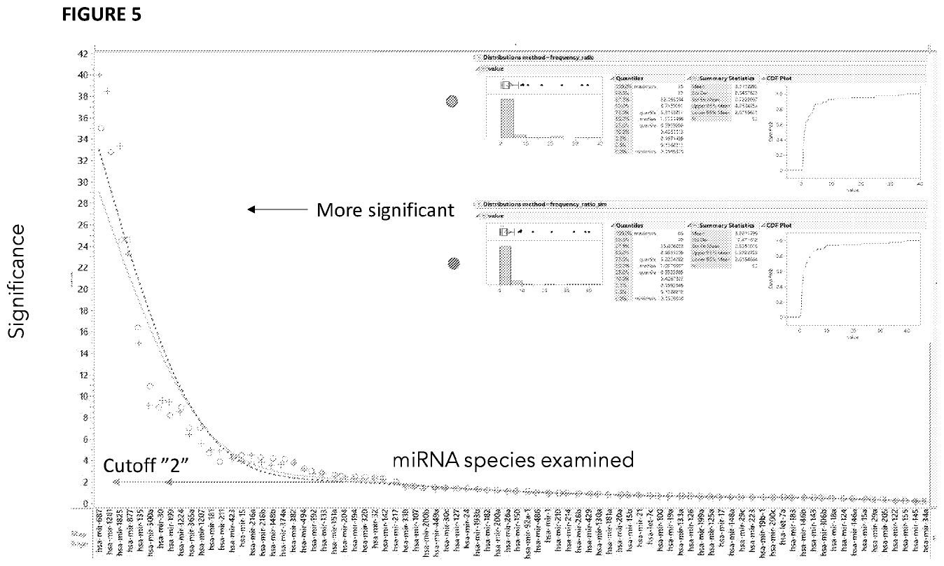

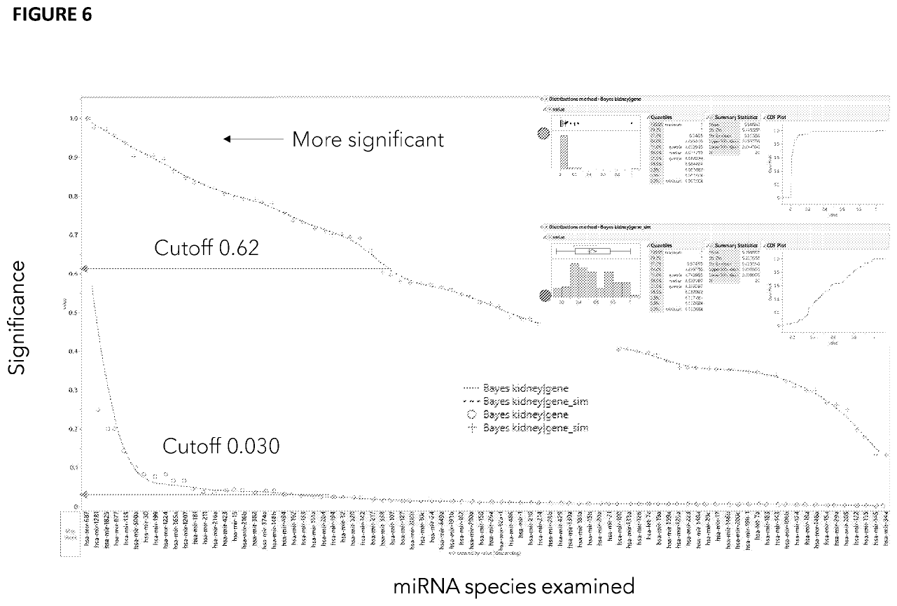

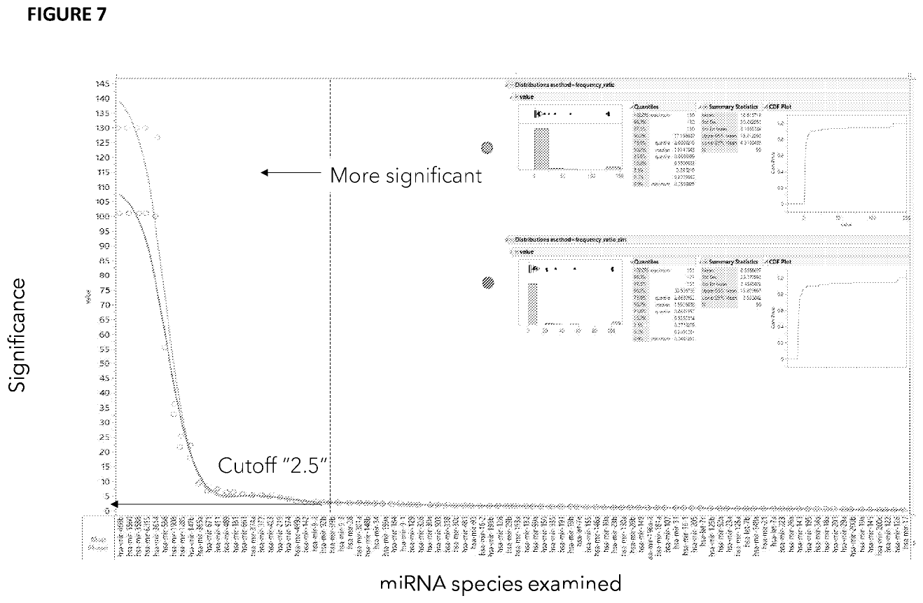

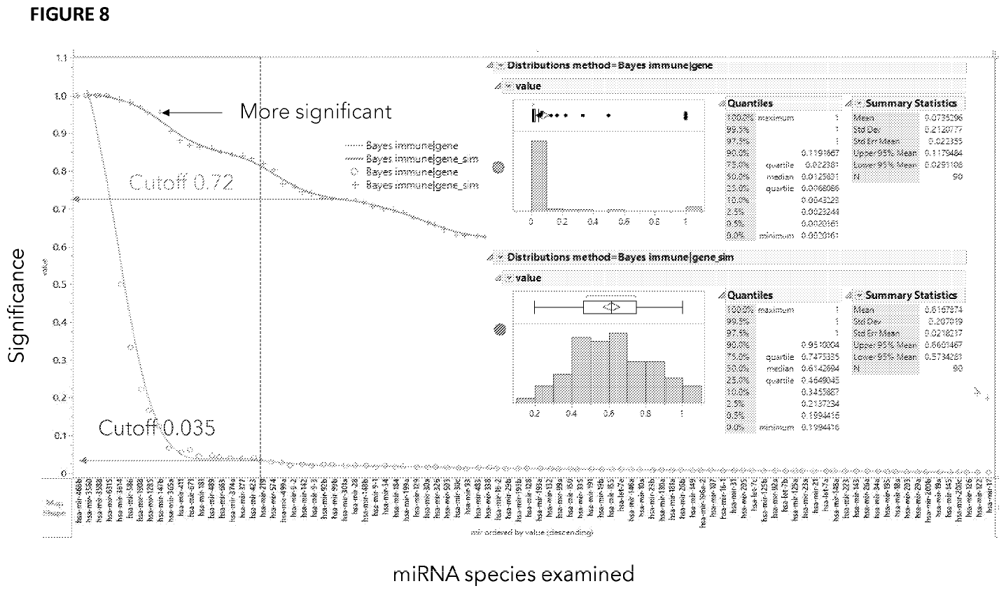

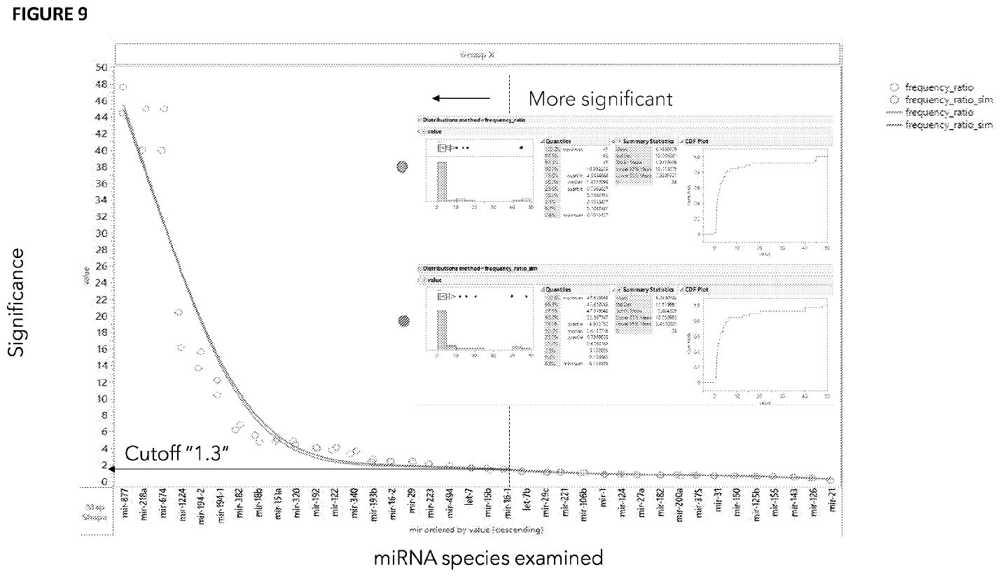

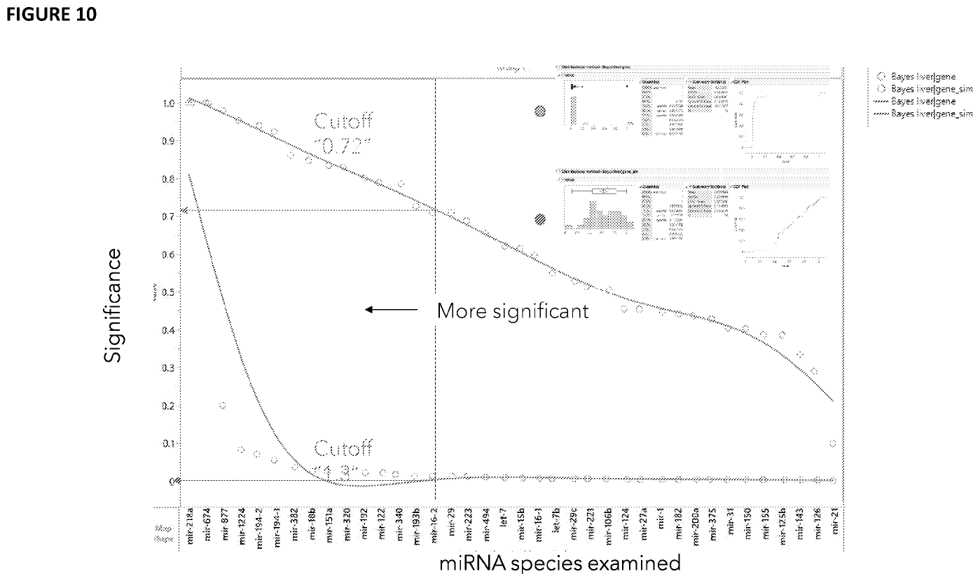

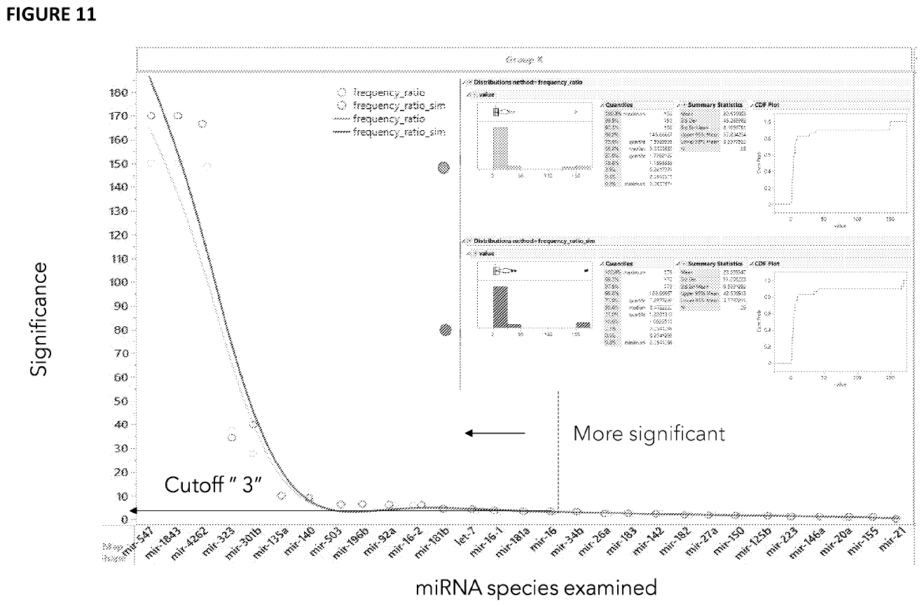

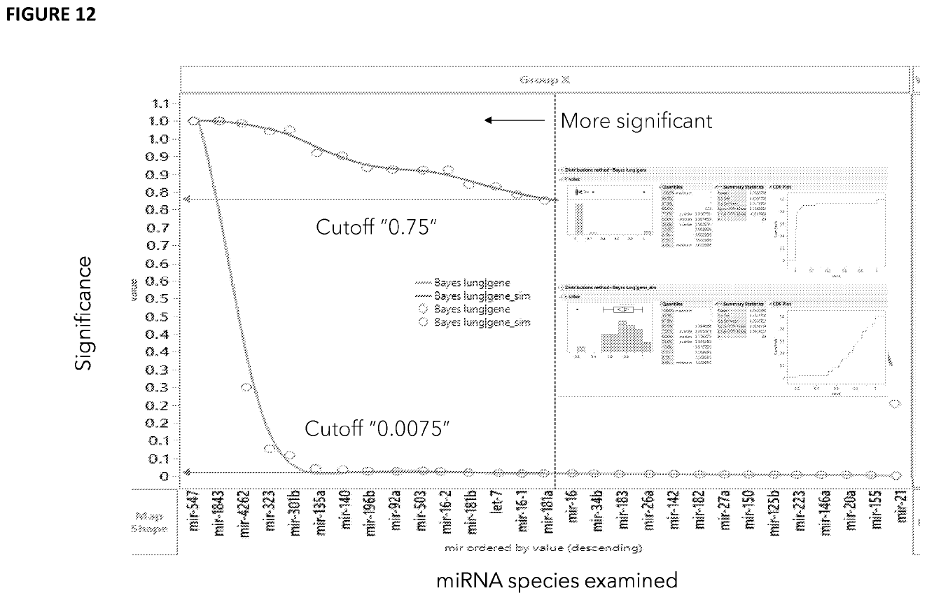

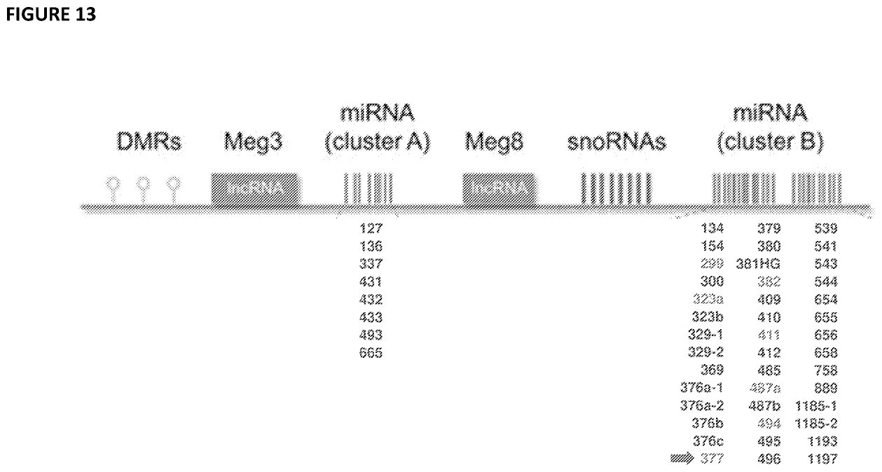

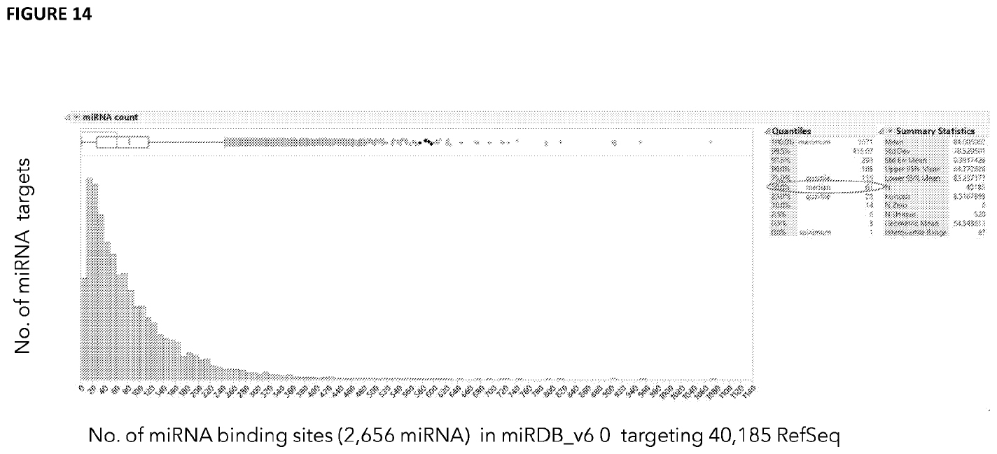

FIGS. 3-14 and Tables 1-3 show exemplified embodiments of this process for various organs and tissues. In some exemplary embodiments, the donor organ is heart and the biomarker comprises hsa-miR-377-3p, hsa-miR-30c-5p, hsa-miR-30d-5p, hsa-miR-195-5p, hsa-miR-208a, hsa-miR-499a-5p, and/or hsa-miR-186-5p.

In some exemplary embodiments, the donor organ is liver and the biomarker comprises hsa-miR-15b-5p, hsa-miR-18b-3p, and/or hsa-miR-223.

In some exemplary embodiments, the donor organ is lung and the biomarker comprises hsa-miR-16-5p.

In some exemplary embodiments, the donor organ is kidney and the biomarker comprises hsa-miR-423-5p, and/or hsa-miR-216a-5p.

In some exemplary embodiments, the biomarker comprises hsa-miR-17-3p.

In some exemplary embodiments, the biomarker comprises hsa-miR-17-3p, hsa-miR-376c-3p, and/or hsa-miR-101-3p.

The set of miRNA targets may be further refined by constraining the miRNAs to those binding mRNA with known organ health relevance.

By using a text mining approach, the inventors of the present disclosure identified the following one or more miRNAs as biomarkers for transplant rejection or organ health: cel-miR-39-3p, hsa-Let-7a-5p, hsa-Let-7d-3p, hsa-Let-7i-5p, hsa-miR-1224-5p, hsa-miR-125b-5p, hsa-miR-126-3p, hsa-miR-1281, hsa-miR-130a-3p, hsa-mir-135a1-5p, hsa-miR-142-3p, hsa-miR-145-3p, hsa-miR-145-5p, hsa-miR-146a-5p, hsa-miR-155-5p, hsa-miR-15a-5p, hsa-miR-15b-5p, hsa-miR-16-5p, hsa-miR-17-3p, hsa-miR-1825, hsa-miR-186-5p, hsa-miR-18a-5p, hsa-miR-18b-3p, hsa-miR-191-5p, hsa-miR-195-5p, hsa-miR-199a-1-3p, hsa-miR-200b-3p, hsa-miR-203a-3p, hsa-miR-204-5p, hsa-miR-208a-3p, hsa-miR-21-5p, hsa-miR-210-3p, hsa-miR-211-5p, hsa-miR-215-5p, hsa-miR-216a-5p, hsa-miR-223-3p, hsa-miR-23a-3p, hsa-miR-24-3p, hsa-miR-299-5p, hsa-miR-30a-3p, hsa-miR-30c-5p, hsa-miR-30d-5p, hsa-miR-320a-3p, hsa-miR-323a-3p, hsa-miR-3615, hsa-miR-377-3p, hsa-miR-378a-3p, hsa-miR-378h, hsa-miR-382-5p, hsa-miR-411-5p, hsa-miR-423-5p, hsa-miR-4286, hsa-miR-449b-5p, hsa-miR-449c-5p, hsa-miR-451a, hsa-miR-484, hsa-miR-487a-5p, hsa-miR-494-3p, hsa-miR-499a-5p, hsa-miR-500a-3p, hsa-miR-625-5p, hsa-miR-877-5p, hsa-miR-92b-3p, hsa-miR-93-5p, hsa-mir 19a-5p, and hsa-mir208a-3p.

In some exemplary embodiments, the target RNA molecules comprise one or more miRNA molecules selected from the group consisting of hsa-miR-92b-5p, hsa-miR-6734-5p, hsa-miR-664a-5p, hsa-miR-576-5p, hsa-miR-539-5p, hsa-miR-500a-5p, hsa-miR-4488, hsa-miR-381-3p, hsa-miR-376c-3p, hsa-miR-339-3p, hsa-miR-185-3p, hsa-miR-17-3p, hsa-miR-1271-5p, and ‘hsa-miR-101.

In some exemplary embodiments, the target RNA molecules comprise one or more miRNA molecules selected from the group consisting of hsa-miR-99b-5p, hsa-miR-660-3p, hsa-miR-500a-5p, hsa-miR-4746-5p, hsa-miR-410-3p, hsa-miR-214-3p, hsa-miR-196b-5p, hsa-miR-194-5p, hsa-miR-192-3p, hsa-miR-185-3p, hsa-miR-17-3p, hsa-miR-136-3p, and hsa-miR-101-3p.

In some exemplary embodiments, the target RNA molecule is hsa-miR-17-3p.

In some exemplary embodiments, the target RNA molecules comprise one or more miRNA molecules selected from the group consisting of hsa-miR-5695, hsa-miR-454-5p, hsa-miR-3912-3p, hsa-miR-363-3p, hsa-miR-27a-5p, hsa-miR-191-5p, hsa-miR-17-3p, hsa-miR-145-5p, and hsa-let-7i-3p.

In some exemplary embodiments, the target RNA molecules comprise one or more miRNA molecules selected from the group consisting of hsa-miR-652-3p, hsa-miR-584-5p, hsa-miR-378a-3p, hsa-miR-338-3p, hsa-miR-320a-3p, hsa-miR-29c-3p, hsa-miR-221-3p, hsa-miR-20a-5p, hsa-miR-199b-3p, hsa-miR-181a-5p, hsa-miR-17-5p, hsa-miR-17-3p, hsa-miR-151a-5p, hsa-miR-151a-3p, hsa-miR-148a-3p, and hsa-miR-143-3p.

In some exemplary embodiments, the target RNA molecules comprise one or more miRNA molecules selected from the group consisting of ‘hsa-miR-576-5p, hsa-miR-539-5p, hsa-miR-500a-5p, hsa-miR-376c-3p, hsa-miR-339-3p, hsa-miR-185-3p, hsa-miR-17-3p, and hsa-miR-101-3p.

In some exemplary embodiments, the target RNA molecules comprise one or more miRNA molecules selected from the group consisting of hsa-miR-17-3p, hsa-miR-500a-5p, hsa-miR-215-5p, hsa-miR-1271-5p, and hsa-miR-151a-5p.

In some exemplary embodiments, the target RNA molecules comprise one or more miRNA molecules selected from the group consisting of hsa-miR-17-3p, hsa-miR-500a-5p, and hsa-miR-151a-5p.

In some exemplary embodiments, the target RNA molecules comprise one or more miRNA molecules selected from the group consisting of cel-miR-39-3p, hsa-Let-7a-5p, hsa-Let-7d-3p, hsa-Let-7i-5p, hsa-miR-1224-5p, hsa-miR-125b-5p, hsa-miR-126-3p, hsa-miR-1281, hsa-miR-130a-3p, hsa-mir-135a1-5p, hsa-miR-142-3p, hsa-miR-145-3p, hsa-miR-145-5p, hsa-miR-146a-5p, hsa-miR-155-5p, hsa-miR-15a-5p, hsa-miR-15b-5p, hsa-miR-16-5p, hsa-miR-17-3p, hsa-miR-1825, hsa-miR-186-5p, hsa-miR-18a-5p, hsa-miR-18b-3p, hsa-miR-191-5p, hsa-miR-195-5p, hsa-miR-199a-1-3p, hsa-miR-200b-3p, hsa-miR-203a-3p, hsa-miR-204-5p, hsa-miR-208a-3p, hsa-miR-21-5p, hsa-miR-210-3p, hsa-miR-211-5p, hsa-miR-215-5p, hsa-miR-216a-5p, hsa-miR-223-3p, hsa-miR-23a-3p, hsa-miR-24-3p, hsa-miR-299-5p, hsa-miR-30a-3p, hsa-miR-30c-5p, hsa-miR-30d-5p, hsa-miR-320a-3p, hsa-miR-323a-3p, hsa-miR-29b-3p, hsa-miR-3615, hsa-miR-377-3p, hsa-miR-378a-3p, hsa-miR-378h, hsa-miR-382-5p, hsa-miR-411-5p, hsa-miR-423-5p, hsa-miR-4286, hsa-miR-449b-5p, hsa-miR-449c-5p, hsa-miR-451a, hsa-miR-484, hsa-miR-487a-5p, hsa-miR-494-3p, hsa-miR-499a-5p, hsa-miR-500a-3p, hsa-miR-625-5p, hsa-miR-877-5p, hsa-miR-92b-3p, hsa-miR-93-5p, hsa-mir19a-5p, hsa-mir208a-3p, hsa-miR-101-3p, hsa-miR-136-3p, hsa-miR-192-3p, hsa-miR-194-5p, hsa-miR-196b-5p, hsa-miR-214-3p, hsa-miR-339-3p, hsa-miR-4746-5p, hsa-miR-500a-5p, hsa-miR-539-5p, hsa-miR-576-5p, hsa-miR-1-3p, hsa-miR-1277-5p, hsa-miR-139-5p, hsa-miR-146b-5p, hsa-miR-183-5p, hsa-miR-188-5p, hsa-miR-190a-5p, hsa-miR-200a-3p, hsa-miR-205-5p, hsa-miR-2115-3p, hsa-miR-3690, hsa-miR-376a-3p, hsa-miR-376b-3p, hsa-miR-412-5p, hsa-miR-449a, hsa-miR-539-3p, hsa-miR-551a, hsa-miR-582-3p, hsa-miR-628-5p, hsa-miR-629-5p, hsa-miR-642a-5p, hsa-miR-651-5p, hsa-miR-873-5p, hsa-miR-887-3p, hsa-miR-376c-3p, hsa-miR-92b-5p, hsa-miR-6734-5p, hsa-miR-664a-5p, hsa-miR-4488, hsa-miR-381-3p, hsa-miR-1271-5p, hsa-miR-101, hsa-miR-99b-5p, hsa-miR-660-3p, hsa-miR-329-3p, hsa-miR-410-3p, hsa-miR-185-3p, hsa-miR-569, hsa-miR-454-5p, hsa-miR-3912-3p, hsa-miR-363-3p, hsa-miR-27a-5p, hsa-miR-191-5p, hsa-miR-145-5p, and hsa-let-7i-3p, ‘hsa-miR-652-3p’, ‘hsa-miR-584-5p, hsa-miR-378a-3p, hsa-miR-338-3p, hsa-miR-29c-3p, hsa-miR-221-3p, hsa-miR-20a-5p, hsa-miR-199b-3p, hsa-miR-181a-5p, hsa-miR-17-5p, hsa-miR-151a-3p, hsa-miR-148a-3p, hsa-miR-143-3p, and hsa-miR-151a-5p.Table 4 shows miRNAs identified as biomarkers for transplant rejection or organ health by using random forest analysis and/or analysis of differential expression of genes of renal or immune origin.

### Tables:

TABLE 1
14q32.31 miRNA cluster

| | In | | | | | |
|---|---|---|---|---|---|---|
| | cluster | | | | | |
| miRNA | 14q32.31 | heart | Kidney | Liver | lung | immune |
| hsa-mir-299 | 1 | 1 | | | | |
| hsa-mir-323 | 1 | | | | 1 | |
| hsa-mir-377 | 2 | 1 | | | | 1 |
| hsa-mir-382 | 2 | | 1 | 1 | | |
| hsa-mir-411 | 1 | | | | | 1 |
| hsa-mir-487 | 1 | 1 | | | | |
| hsa-mir-494 | 2 | | 1 | 1 | | |
| All miRNA | 10 | 49 | 27 | 21 | 17 | 22 |

TABLE 2
16 -of-111 higher specificity miRNA also bind to 4-of-5 CareDx messenger
RNA genes: miRNA Binding score >60. Similar but not identical mirRNA
predicted results b/n different miRNA target prediction algorithms

| Target | Target | miRNA | Gene | mir | Found in our | In cluster | mirBAse | coord | coord |
|---|---|---|---|---|---|---|---|---|---|
| Rank | Score | Name | Symbol | Name | target search | 14q32.31 ? | coordinates | 1 | 2 |
| 1 | 98 | hsa-miR-377-3p | DCAF12 | hsa-mir-377 | heart | 14q32.31 | chr14 | 101062094 | 101062115 |
| | | | | | | miRNA | | | |
| | | | | | | cluster B | | | |
| 24 | 84 | hsa-miR-15b-5p | FLT3 | hsa-mir-15b | Liver | | chr3 | 160404607 | 160404628 |
| 26 | 84 | hsa-miR-30c-5p | 8-Mar | hsa-mir-30c | heart | | chr1 | 40757300 | 40757322 |
| 27 | 84 | hsa-miR-16-5p | FLT3 | hsa-mir-16 | lung | | chr13 | 50049027 | 50049048 |
| 30 | 84 | hsa-miR-30d-5p | 8-Mar | hsa-mir-30d | heart | | chr8 | 134804919 | 134804940 |
| 32 | 84 | hsa-miR-195-5p | FLT3 | hsa-mir-195 | heart | | chr17 | 7017667 | 7017687 |
| 38 | 83 | hsa-miR-18b-3p | FLT3 | hsa-mir-18b | Liver | | chrX | 134170042 | 134170063 |
| 49 | 78 | hsa-miR-208a-3p | DCAF12 | hsa-mir-208a | heart | | chr14 | 23388602 | 23388623 |
| 59 | 76 | hsa-miR-15b-5p | PDCD1 | hsa-mir-15b | Liver | | chr3 | 160404607 | 160404628 |
| 60 | 76 | hsa-miR-16-5p | PDCD1 | hsa-mir-16 | lung | | chr13 | 50049027 | 50049048 |
| 61 | 76 | hsa-miR-223-3p | DCAF12 | hsa-mir-223 | Liver | | chrX | 66018937 | 66018958 |
| 63 | 76 | hsa-miR-195-5p | PDCD1 | hsa-mir-195 | heart | | chr17 | 7017667 | 7017687 |
| 77 | 73 | hsa-miR-423-5p | PDCD1 | hsa-mir-423 | Kidney | | chr17 | 30117095 | 30117117 |
| 83 | 71 | hsa-miR-499a-5p | DCAF12 | hsa-mir-499a | heart | | chr20 | 34990408 | 34990428 |
| 87 | 69 | hsa-miR-186-5p | 8-Mar | hsa-mir-186 | heart | | chr1 | 71067681 | 71067702 |
| 98 | 63 | hsa-miR-216a-5p | DCAF12 | hsa-mir-216a | Kidney | | chr2 | 55989020 | 55989041 |

TABLE 3

| | Gene count | | | | Weighted scores | | | In target scan |
|---|---|---|---|---|---|---|---|---|
| miRNA | DCAF12 | FLT3 | PDCD1 | count | DCAF12 | FLT3 | PDCD1 | and MIRdb |
| hsa-miR-499a-5p | 1 | | | 1 | 94 | | | hsa-miR-499a-5p |
| hsa-miR-377-3p | 1 | | | 1 | 93 | | | hsa-miR-377-3p |
| hsa-miR-216a-5p | 1 | | | 1 | 97 | | | hsa-miR-216a-5p |
| hsa-miR-208a-3p | 1 | | | 1 | 99 | | | hsa-miR-208a-3p |
| hsa-miR-195-5p | | 1 | 1 | 2 | | 98 | 90 | hsa-miR-195-5p |
| hsa-miR-16-5p | | 1 | 1 | 2 | | 98 | 90 | hsa-miR-16-5p |
| hsa-miR-15b-5p | | 1 | 1 | 2 | | 97 | 90 | hsa-miR-15b-5p |

TABLE 4

| miRNA | Source |
|---|---|
| hsa-miR-101-3p | RFE-Random Forest feature selection |
| hsa-miR-136-3p | RFE-Random Forest feature selection |
| hsa-miR-17-3p | RFE-Random Forest feature selection |
| hsa-miR-185-3p | RFE-Random Forest feature selection |
| hsa-miR-192-3p | RFE-Random Forest feature selection |
| hsa-miR-194-5p | RFE-Random Forest feature selection |
| hsa-miR-196b-5p | RFE-Random Forest feature selection |
| hsa-miR-214-3p | RFE-Random Forest feature selection |
| hsa-miR-339-3p | RFE-Random Forest feature selection |
| hsa-miR-4746-5p | RFE-Random Forest feature selection |
| hsa-miR-500a-5p | RFE-Random Forest feature selection |
| hsa-miR-539-5p | RFE-Random Forest feature selection |
| hsa-miR-576-5p | RFE-Random Forest feature selection |
| hsa-miR-1-3p | Differentially expressed genes of |
| | renal or immune origin |
| hsa-miR-1277-5p | Differentially expressed genes of |
| | renal or immune origin |
| hsa-miR-139-5p | Differentially expressed genes of |
| | renal or immune origin |
| hsa-miR-146b-5p | Differentially expressed genes of |
| | renal or immune origin |
| hsa-miR-183-5p | Differentially expressed genes of |
| | renal or immune origin |
| hsa-miR-188-5p | Differentially expressed genes of |
| | renal or immune origin |
| hsa-miR-190a-5p | Differentially expressed genes of |
| | renal or immune origin |
| hsa-miR-195-5p | Differentially expressed genes of |
| | renal or immune origin |
| hsa-miR-200a-3p | Differentially expressed genes of |
| | renal or immune origin |
| hsa-miR-205-5p | Differentially expressed genes of |
| | renal or immune origin |
| hsa-miR-2115-3p | Differentially expressed genes of |
| | renal or immune origin |
| hsa-miR-215-5p | Differentially expressed genes of |
| | renal or immune origin |
| hsa-miR-223-3p | Differentially expressed genes of |
| | renal or immune origin |
| hsa-miR-29b-3p | Differentially expressed genes of |
| | renal or immune origin |
| hsa-miR-329-3p | Differentially expressed genes of |
| | renal or immune origin |
| hsa-miR-3690 | Differentially expressed genes of |
| | renal or immune origin |
| hsa-miR-376a-3p | Differentially expressed genes of |
| | renal or immune origin |
| hsa-miR-376b-3p | Differentially expressed genes of |
| | renal or immune origin |
| hsa-miR-412-5p | Differentially expressed genes of |
| | renal or immune origin |
| hsa-miR-449a | Differentially expressed genes of |
| | renal or immune origin |
| hsa-miR-449c-5p | Differentially expressed genes of |
| | renal or immune origin |
| hsa-miR-539-3p | Differentially expressed genes of |
| | renal or immune origin |
| hsa-miR-551a | Differentially expressed genes of |
| | renal or immune origin |
| hsa-miR-582-3p | Differentially expressed genes of |
| | renal or immune origin |
| hsa-miR-628-5p | Differentially expressed genes of |
| | renal or immune origin |
| hsa-miR-629-5p | Differentially expressed genes of |
| | renal or immune origin |
| hsa-miR-642a-5p | Differentially expressed genes of |
| | renal or immune origin |
| hsa-miR-651-5p | Differentially expressed genes of |
| | renal or immune origin |
| hsa-miR-873-5p | Differentially expressed genes of |
| | renal or immune origin |
| hsa-miR-887-3p | Differentially expressed genes of |
| | renal or immune origin |
| hsa-miR-376c-3p | Both (RFE-Random Forest feature |
| | selection & Differentially expressed |
| | genes of renal or immune origin) |

1. A method of preparing a composition of amplified nucleic acids derived from a sample obtained from a transplant recipient useful for determining transplant rejection, comprising:
a) extracting fragmented or intact RNA derived from the sample obtained from the transplant recipient, wherein the extracted RNA comprises donor- and/or recipient-derived RNA;
b) preparing a composition of amplified nucleic acids from the RNA by performing reverse transcription of the RNA to produce complementary DNA (cDNA) followed by multiplex targeted amplification to obtain amplicons of a plurality of target RNA molecules in a single reaction mixture, wherein the plurality of target RNA molecules is indicative of transplant rejection or organ health.

2. (canceled)

3. The method of claim 1, wherein determining transplant rejection comprises measuring an amount of the plurality of target RNA molecules in the sample based on the amplicons obtained in step b) to quantify an amount of donor-derived target RNA molecules, and determining whether the amount of donor derived target RNA molecules or a function thereof exceeds a cutoff threshold indicating transplant rejection.

4. The method of claim 1, further comprising utilizing CRISPR-Cas to target and deplete contaminating or overabundant nucleic acid species in the sample, thereby increasing the fraction of desired reads that map to a target loci of interest per sample and the sample throughput per sequencing run

5. The method of claim 4, the sample comprises whole blood or hemolysis tainted blood, serum or plasma samples, and wherein a plurality of guide RNAs are used to target a plurality of loci in the same reaction to deplete contaminating or overabundant nucleic acid species, thereby increasing a detection rate of the target loci of interest.

6. The method of claim 4, wherein the contaminating or overabundant nucleic acid species comprise hemoglobin mRNA, tRNA, and/or rRNA; and/or_miR-451, miR-144, and/or miR-486.

7. The method of claim 1, further comprising depleting adaptor dimers, primer dimers, unwanted ligation products from the composition of amplified nucleic acids comprising target loci, thereby increasing the fraction of desired reads that map to a target loci of interest per sample and the sample throughput per sequencing run.

8. The method of claim 4, wherein Cas9/Cas12a is utilized to (i) remove nucleic acid species after reverse transcription of RNA and before multiplex amplification, or (ii) to remove nucleic acid species after 1-10 cycles of multiplex amplification of the complementary DNA.

9. (canceled)

10. The method of claim 4, wherein the contaminating or overabundant nucleic acid species are RNA, and wherein Cas13 is used to remove the contaminating or overabundant RNA species from the sample.

11. The method of claim 1, further comprising:
(c) measuring the amount of donor-derived cell-free DNA in a sample obtained from the transplant recipient, extracting cell-free DNA from the sample obtained from the transplant recipient, wherein the extracted cell-free DNA comprises donor-derived cell-free DNA and recipient-derived cell-free DNA;
(d) performing targeted amplification of the extracted DNA at 50-50,000 target loci in a single reaction volume;
(e) sequencing the amplified DNA by high-throughput sequencing to obtain sequencing reads and quantifying the amount of donor-derived cell-free DNA based on the sequencing reads, determining transplant rejection based on whether the amount of donor-derived cell-free DNA or a function thereof exceeds a cutoff threshold of cell-free DNA amount that indicates transplant rejection, wherein transplant rejection is determined based on whether both the amount of donor-derived RNA and the amount of donor derived cell-free DNA or function thereof exceeds a cutoff threshold that indicates transplant rejection.

12. (canceled)

13. The method of claim 1, wherein the sample comprises blood, plasma, serum, cerebral spinal fluid (CSF), or urine.

14. The method of claim 1, wherein the transplant recipient is a human subject.

15. The method of claim 1, wherein the transplant recipient has received one or more transplanted organs selected from pancreas, kidney, liver, lung, heart, intestinal, thymus or uterus.

16. The method of claim 1, wherein the transplant recipient has: (i) received one or more transplanted organs from the same transplant donors, (ii) received one or more transplanted organs from multiple different transplant donors, or (iii) received simultaneous transplantation of more than one organ.

17. (canceled)

18. (canceled)

19. (canceled)

20. (canceled)

21. The method of claim 3, further comprising measuring the amount of the plurality of target RNA molecules longitudinally for the same transplant recipient; to determine a longitudinal change in the amount of RNA.

22. The method of claim 1, wherein the RNA is (i) cell-free RNA, (ii) small messenger RNA (mRNA), and/or (iii) small noncoding RNA (sncRNA).

23. The method of claim 22, wherein the cell-free RNA is derived from exosomes or microvesicles.

24. (canceled)

25. (canceled)

26. (canceled)

27. (canceled)

28. The method of claim 22, wherein the sncRNA comprises micro RNA (miRNA), piwi-interacting RNA (piRNA), small nucleolar RNA (snoRNA), small nuclear RNA (snRNA), or miscellaneous RNA (miscRNA).

29. (canceled)

30. The method of claim 1, wherein step (b) comprises: (i) amplification of at least 2, at least 5, at least 10, at least 20, at least 30, at least 50, at least 100 target RNA molecules; or (ii) multiplex amplification of at least 100, at least 500, at least 1000, at least 2000 target loci, from 10-1000, 100-10000, 50-50000, or 500-20000 target loci.

31. The method of claim 1, wherein the amount of RNA or cell-free DNA is measured by: (i) a quantitative PCR method, and wherein the quantitative PCR method comprises real-time PCR or digital PCR, (ii) massively multiplex PCR (mmPCR) to obtain amplicons comprising biomarkers, and sequencing of the amplicons, (iii) using microarray, (iv) using molecular barcodes and microscopic imaging.

32. (canceled)

33. (canceled)

34. (canceled)

35. The method of claim 1, wherein the cutoff threshold is: (i) an estimated percentage of donor-derived RNA out of total RNA or a function thereof, (ii) an estimated percentage of donor-derived cell-free DNA out of total cell-free DNA or a functional thereof, or (iii) is proportional to an absolute donor-derived RNA or cell-free DNA concentration.

36. (canceled)

37. (canceled)

38. The method of claim 1, wherein a rejection risk for the transplant recipient can be determined based on the amount of donor-derived RNA and/or an amount of cell-free DNA by using machine learning analysis.

39. (canceled)

40. (canceled)

41. (canceled)

42. A method of preparing a composition of nucleic acids derived from a sample obtained from a transplant recipient useful for determination of transplant rejection, comprising:
(a) extracting RNA from the sample obtained from the transplant recipient;
(b) preparing a composition of nucleic acids from the extracted RNA, wherein the nucleic acids comprise a plurality of biomarkers indicative of transplant rejection;
(c) measuring the amount of RNA comprising a plurality of biomarker indicative of transplant rejection in the sample obtained from the transplant recipient; and
(d) determining whether the amount of recipient-derived RNA comprising biomarkers indicative of transplant rejection exceeds a cutoff threshold or a function thereof.

43. (canceled)

44. (canceled)

45. The method of claim 42, wherein the nucleic acids comprising the plurality of biomarkers indicative of transplant rejection are determined by hybridization of the extracted RNA to a plurality of probes, or by reverse transcription and targeted amplification of the extracted RNA in a single reaction volume, and sequencing the amplified RNA by high-throughput sequencing to obtain sequencing reads.

46. The method of claim 42, the RNA comprises biomarkers of an increased immune response, or a decreased immune response.

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. The method of claim 1, wherein the RNA is:
(i) miRNA determined to be of relevance to transplant organ health determined by text mining databases;
(ii) miRNA predicted to bind mRNAs known to be relevant for transplant organ health;
(iii) miRNA determined to be of relevance to transplant organ health determined by text mining databases, and predicted to bind mRNAs known to be relevant for transplant organ health;
(iv) one or more miRNA molecules selected from the group consisting of cel-miR-39-3p, hsa-Let-7a-5p, hsa-Let-7d-3p, hsa-Let-7i-5p, hsa-miR-1224-5p, hsa-miR-125b-5p, hsa-miR-126-3p, hsa-miR-1281, hsa-miR-130a-3p, hsa-mir-135a1-5p, hsa-miR-142-3p, hsa-miR-145-3p, hsa-miR-145-5p, hsa-miR-146a-5p, hsa-miR-155-5p, hsa-miR-15a-5p, hsa-miR-15b-5p, hsa-miR-16-5p, hsa-miR-17-3p, hsa-miR-1825, hsa-miR-186-5p, hsa-miR-18a-5p, hsa-miR-18b-3p, hsa-miR-191-5p, hsa-miR-195-5p, hsa-miR-199a-1-3p, hsa-miR-200b-3p, hsa-miR-203a-3p, hsa-miR-204-5p, hsa-miR-208a-3p, hsa-miR-21-5p, hsa-miR-210-3p, hsa-miR-211-5p, hsa-miR-215-5p, hsa-miR-216a-5p, hsa-miR-223-3p, hsa-miR-23a-3p, hsa-miR-24-3p, hsa-miR-299-5p, hsa-miR-30a-3p, hsa-miR-30c-5p, hsa-miR-30d-5p, hsa-miR-320a-3p, hsa-miR-323a-3p, hsa-miR-3615, hsa-miR-377-3p, hsa-miR-378a-3p, hsa-miR-378h, hsa-miR-382-5p, hsa-miR-411-5p, hsa-miR-423-5p, hsa-miR-4286, hsa-miR-449b-5p, hsa-miR-449c-5p, hsa-miR-451a, hsa-miR-484, hsa-miR-487a-5p, hsa-miR-494-3p, hsa-miR-499a-5p, hsa-miR-500a-3p, hsa-miR-625-5p, hsa-miR-877-5p, hsa-miR-92b-3p, hsa-miR-93-5p, hsa-mir19a-5p, and hsa-mir 208a-3p;
(v) one or more miRNA molecules selected from the group consisting of cel-miR-39-3p, hsa-Let-7a-5p, hsa-Let-7d-3p, hsa-Let-7i-5p, hsa-miR-1224-5p, hsa-miR-125b-5p, hsa-miR-126-3p, hsa-miR-1281, hsa-miR-130a-3p, hsa-mir-135a1-5p, hsa-miR-142-3p, hsa-miR-145-3p, hsa-miR-145-5p, hsa-miR-146a-5p, hsa-miR-155-5p, hsa-miR-15a-5p, hsa-miR-15b-5p, hsa-miR-16-5p, hsa-miR-17-3p, hsa-miR-1825, hsa-miR-186-5p, hsa-miR-18a-5p, hsa-miR-18b-3p, hsa-miR-191-5p, hsa-miR-195-5p, hsa-miR-199a-1-3p, hsa-miR-200b-3p, hsa-miR-203a-3p, hsa-miR-204-5p, hsa-miR-208a-3p, hsa-miR-21-5p, hsa-miR-210-3p, hsa-miR-211-5p, hsa-miR-215-5p, hsa-miR-216a-5p, hsa-miR-223-3p, hsa-miR-23a-3p, hsa-miR-24-3p, hsa-miR-299-5p, hsa-miR-30a-3p, hsa-miR-30c-5p, hsa-miR-30d-5p, hsa-miR-320a-3p, hsa-miR-323a-3p, hsa-miR-3615, hsa-miR-377-3p, hsa-miR-378a-3p, hsa-miR-378h, hsa-miR-382-5p, hsa-miR-411-5p, hsa-miR-423-5p, hsa-miR-4286, hsa-miR-449b-5p, hsa-miR-449c-5p, hsa-miR-451a, hsa-miR-484, hsa-miR-487a-5p, hsa-miR-494-3p, hsa-miR-499a-5p, hsa-miR-500a-3p, hsa-miR-625-5p, hsa-miR-877-5p, hsa-miR-92b-3p, hsa-miR-93-5p, hsa-mir19a-5p, hsa-mir208a-3p, hsa-miR-101-3p, hsa-miR-136-3p, hsa-miR-185-3p, hsa-miR-192-3p, hsa-miR-194-5p, hsa-miR-196b-5p, hsa-miR-214-3p, hsa-miR-339-3p, hsa-miR-4746-5p, hsa-miR-500a-5p, hsa-miR-539-5p, hsa-miR-576-5p, hsa-miR-1-3p, hsa-miR-1277-5p, hsa-miR-139-5p, hsa-miR-146b-5p, hsa-miR-183-5p, hsa-miR-188-5p, hsa-miR-190a-5p, hsa-miR-200a-3p, hsa-miR-205-5p, hsa-miR-2115-3p, hsa-miR-329-3p, hsa-miR-3690, hsa-miR-376a-3p, hsa-miR-376b-3p, hsa-miR-412-5p, hsa-miR-449a, hsa-miR-539-3p, hsa-miR-551a, hsa-miR-582-3p, hsa-miR-628-5p, hsa-miR-629-5p, hsa-miR-642a-5p, hsa-miR-651-5p, hsa-miR-873-5p, hsa-miR-887-3p, and hsa-miR-376c-3p;
(vi) one or more miRNA molecules selected from the group consisting of hsa-miR-101-3p, hsa-miR-136-3p, hsa-miR-17-3p, hsa-miR-185-3p, hsa-miR-192-3p, hsa-miR-194-5p, hsa-miR-196b-5p, hsa-miR-214-3p, hsa-miR-339-3p, hsa-miR-4746-5p, hsa-miR-500a-5p, hsa-miR-539-5p, hsa-miR-576-5p, hsa-miR-1-3p, hsa-miR-1277-5p, hsa-miR-139-5p, hsa-miR-146b-5p, hsa-miR-183-5p, hsa-miR-188-5p, hsa-miR-190a-5p, hsa-miR-195-5p, hsa-miR-200a-3p, hsa-miR-205-5p, hsa-miR-2115-3p, hsa-miR-215-5p, hsa-miR-223-3p, hsa-miR-29b-3p, hsa-miR-329-3p, hsa-miR-3690, hsa-miR-376a-3p, hsa-miR-376b-3p, hsa-miR-412-5p, hsa-miR-449a, hsa-miR-449c-5p, hsa-miR-539-3p, hsa-miR-551a, hsa-miR-582-3p, hsa-miR-628-5p, hsa-miR-629-5p, hsa-miR-642a-5p, hsa-miR-651-5p, hsa-miR-873-5p, hsa-miR-887-3p, and hsa-miR-376c-3p;
(vii) one or more miRNA molecules selected from the group consisting of hsa-miR-92b-5p, hsa-miR-6734-5p, hsa-miR-664a-5p, hsa-miR-576-5p, hsa-miR-539-5p, hsa-miR-500a-5p, hsa-miR-4488, hsa-miR-381-3p, hsa-miR-376c-3p, hsa-miR-339-3p, hsa-miR-185-3p, hsa-miR-17-3p, hsa-miR-1271-5p, and ‘hsa-miR-101;
(viii) one or more miRNA molecules selected from the group consisting of hsa-miR-99b-5p, hsa-miR-660-3p, hsa-miR-500a-5p, hsa-miR-4746-5p, hsa-miR-410-3p, hsa-miR-214-3p, hsa-miR-196b-5p, hsa-miR-194-5p, hsa-miR-192-3p, hsa-miR-185-3p, hsa-miR-17-3p, hsa-miR-136-3p, and hsa-miR-101-3p;
(ix) hsa-miR-17-3p:
(x) one or more miRNA molecules selected from the group consisting of hsa-miR-5695, hsa-miR-454-5p, hsa-miR-3912-3p, hsa-miR-363-3p, hsa-miR-27a-5p, hsa-miR-191-5p, hsa-miR-17-3p, hsa-miR-145-5p, and hsa-let-71-3p;
(xi) one or more miRNA molecules selected from the group consisting of hsa-miR-652-3p, hsa-miR-584-5p, hsa-miR-378a-3p, hsa-miR-338-3p, hsa-miR-320a-3p, hsa-miR-29c-3p, hsa-miR-221-3p, hsa-miR-20a-5p, hsa-miR-199b-3p, hsa-miR-181a-5p, hsa-miR-17-5p, hsa-miR-17-3p, hsa-miR-151a-5p, hsa-miR-151a-3p, hsa-miR-148a-3p, and hsa-miR-143-3p;
(xii) one or more miRNA molecules selected from the group consisting of ‘hsa-miR-576-5p, hsa-miR-539-5p, hsa-miR-500a-5p, hsa-miR-376c-3p, hsa-miR-339-3p, hsa-miR-185-3p, hsa-miR-17-3p, and hsa-miR-101-3p;
(xiii) one or more miRNA molecules selected from the group consisting of hsa-miR-17-3p, hsa-miR-500a-5p, hsa-miR-215-5p, hsa-miR-1271-5p, and hsa-miR-151a-5p;
(xiv) one or more miRNA molecules selected from the group consisting of hsa-miR-17-3p, hsa-miR-500a-5p, and hsa-miR-151a-5p; or
(xv) one or more miRNA molecules selected from the group consisting of cel-miR-39-3p, hsa-Let-7a-5p, hsa-Let-7d-3p, hsa-Let-7i-5p, hsa-miR-1224-5p, hsa-miR-125b-5p, hsa-miR-126-3p, hsa-miR-1281, hsa-miR-130a-3p, hsa-mir-135a1-5p, hsa-miR-142-3p, hsa-miR-145-3p, hsa-miR-145-5p, hsa-miR-146a-5p, hsa-miR-155-5p, hsa-miR-15a-5p, hsa-miR-15b-5p, hsa-miR-16-5p, hsa-miR-17-3p, hsa-miR-1825, hsa-miR-186-5p, hsa-miR-18a-5p, hsa-miR-18b-3p, hsa-miR-191-5p, hsa-miR-195-5p, hsa-miR-199a-1-3p, hsa-miR-200b-3p, hsa-miR-203a-3p, hsa-miR-204-5p, hsa-miR-208a-3p, hsa-miR-21-5p, hsa-miR-210-3p, hsa-miR-211-5p, hsa-miR-215-5p, hsa-miR-216a-5p, hsa-miR-223-3p, hsa-miR-23a-3p, hsa-miR-24-3p, hsa-miR-299-5p, hsa-miR-30a-3p, hsa-miR-30c-5p, hsa-miR-30d-5p, hsa-miR-320a-3p, hsa-miR-323a-3p, hsa-miR-29b-3p, hsa-miR-3615, hsa-miR-377-3p, hsa-miR-378a-3p, hsa-miR-378h, hsa-miR-382-5p, hsa-miR-411-5p, hsa-miR-423-5p, hsa-miR-4286, hsa-miR-449b-5p, hsa-miR-449c-5p, hsa-miR-451a, hsa-miR-484, hsa-miR-487a-5p, hsa-miR-494-3p, hsa-miR-499a-5p, hsa-miR-500a-3p, hsa-miR-625-5p, hsa-miR-877-5p, hsa-miR-92b-3p, hsa-miR-93-5p, hsa-mir19a-5p, hsa-mir208a-3p, hsa-miR-101-3p, hsa-miR-136-3p, hsa-miR-192-3p, hsa-miR-194-5p, hsa-miR-196b-5p, hsa-miR-214-3p, hsa-miR-339-3p, hsa-miR-4746-5p, hsa-miR-500a-5p, hsa-miR-539-5p, hsa-miR-576-5p, hsa-miR-1-3p, hsa-miR-1277-5p, hsa-miR-139-5p, hsa-miR-146b-5p, hsa-miR-183-5p, hsa-miR-188-5p, hsa-miR-190a-5p, hsa-miR-200a-3p, hsa-miR-205-5p, hsa-miR-2115-3p, hsa-miR-3690, hsa-miR-376a-3p, hsa-miR-376b-3p, hsa-miR-412-5p, hsa-miR-449a, hsa-miR-539-3p, hsa-miR-551a, hsa-miR-582-3p, hsa-miR-628-5p, hsa-miR-629-5p, hsa-miR-642a-5p, hsa-miR-651-5p, hsa-miR-873-5p, hsa-miR-887-3p, hsa-miR-376c-3p, hsa-miR-92b-5p, hsa-miR-6734-5p, hsa-miR-664a-5p, hsa-miR-4488, hsa-miR-381-3p, hsa-miR-1271-5p, hsa-miR-101, hsa-miR-99b-5p, hsa-miR-660-3p, hsa-miR-329-3p, hsa-miR-410-3p, hsa-miR-185-3p, hsa-miR-569, hsa-miR-454-5p, hsa-miR-3912-3p, hsa-miR-363-3p, hsa-miR-27a-5p, hsa-miR-191-5p, hsa-miR-145-5p, and hsa-1et-7i-3p, ‘hsa-miR-652-3p’, ‘hsa-miR-584-5p, hsa-miR-378a-3p, hsa-miR-338-3p, hsa-miR-29c-3p, hsa-miR-221-3p, hsa-miR-20a-5p, hsa-miR-199b-3p, hsa-miR-181a-5p, hsa-miR-17-5p, hsa-miR-151a-3p, hsa-miR-148a-3p, hsa-miR-143-3p, and hsa-miR-151a-5p.

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)
